(19) United States
(12) Patent Application Publication
(10) Pub. No.: US 2024/0166684 A1
(43) Pub. Date: May 23, 2024

(54) COMPOUNDS FOR ALTERING LEVELS OF ONE OR MORE NKA ALPHA SUBUNITS AND THEIR USE IN TREATING PRION DISEASES OR BRAIN DISEASES ASSOCIATED WITH CELLULAR PRION PROTEIN

(71) Applicants: Gerold Schmitt-Ulms, Toronto (CA); Mohadeseh Mehrabian, Richmond Hill (CA); Declan Gwillym Williams, Toronto (CA)

(72) Inventors: Gerold Schmitt-Ulms, Toronto (CA); Mohadeseh Mehrabian, Richmond Hill (CA); Declan Gwillym Williams, Toronto (CA)

(21) Appl. No.: 18/280,977

(22) PCT Filed: Mar. 10, 2022
(86) PCT No.: PCT/CA2022/050358

§ 371 Date: Sep. 8, 2023

Related U.S. Application Data
(60) Provisional application No. 63/159,289, filed on Mar. 10, 2021.

(51) Int. Cl. C07J 19/00; A61P 25/00

(52) U.S. Cl. CPC C07J 19/005; A61P 25/00

(57) ABSTRACT

The present application relates to methods for treating and/or preventing prion diseases and/or brain diseases, disorders or conditions that benefit from reduced levels of the cellular prion protein (PrPC) and/or altered levels of NKA alpha subunits comprising administering and effective amount of one or more agents that alter levels of one or more NKA alpha subunits, in a subject in need thereof, wherein each NKA alpha subunit with altered levels is a different paralog. The one or more agents are, for example, one or more compounds of Formula I, or a pharmaceutically acceptable salt and/or solvate thereof.

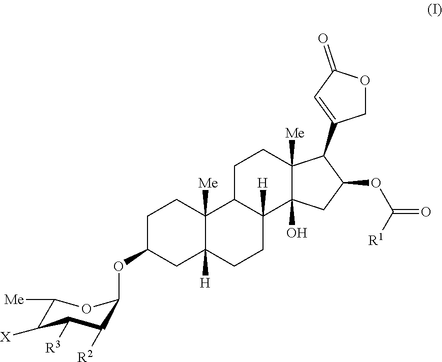

### RELATED APPLICATIONS

The present application claims the benefit of priority of co-pending U.S. provisional patent application No. 63/159,289 filed on Mar. 10, 2021, the contents of which are incorporated herein by reference in their entirety.

### FIELD

The present application relates to methods of treating and/or preventing prion diseases and/or other brain diseases, including brain diseases, disorders or conditions associated that benefit from reduced levels of the cellular prion protein (PrP^{C}) and/or altered levels of NKA alpha subunits. More particularly, it relates methods for treating and/or preventing prion diseases and/or other brain diseases, disorders or conditions that benefit from reduced levels of the cellular prion protein (PrP^{C}) and/or altered levels of NKA alpha subunits using one or more agents that alter levels of one or more NKA alpha subunit paralogs, such as the compounds disclosed herein.

### BACKGROUND

The cellular prion protein (PrP^{C}) is widely understood to be the substrate of a conformational conversion that causes a group of neurodegenerative diseases, known as prion diseases (Prusiner, 1998). Several lines of evidence also point toward PrP^{C} as a receptor for the cellular docking of oligomeric Aβ peptides that mediate toxicity in Alzheimer's disease (Lauren et al., 2009).

Prion diseases are incurable neurodegenerative diseases in humans and livestock. The etiology of these devastating diseases centers on the prion protein, whose normal cellular form (PrP^{C}) is expressed in most vertebrate cells. In the disease, PrP^{C} undergoes a conformational change to PrP^{Sc} that endows distinct physicochemical properties to the protein. PrP^{Sc} can seed the conversion of other PrP^{C} molecules through templated conversion, thereby causing the accumulation of PrP^{Sc} and the spread of the disease (Prusiner, 1998). Insights into how the accumulation of PrP^{Sc} causes toxicity to brain cells has remained spotty. Because PrP-deficient cells and mice exhibit no overt phenotypes (Bueler et al., 1992), it is widely assumed that the toxicity of PrP^{Sc} involves a gain-of-toxic function (Tatzelt & Schatzl, 2007). However, it is also apparent that the expression of PrP^{C} is critical for the cellular toxicity to manifest (Brandner et al., 1996). Consequently, ongoing research aims to decipher the molecular mechanism by which PrP^{C} contributes to the disease with a view to block the toxicity of PrP^{Sc}.

Experimental or natural heterozygosity of the prion gene (Prnp) does not appear to confer robust phenotypes and even its complete deficiency causes only minor phenotypes (Bueler et al., 1992). Consistent with these experimental observations from studies in mice, human 23andMe data indicate that individuals with only one functional PRNP allele can reach advanced age (Minikel et al., 2016).

Mice lacking the Prnp gene are refractory to prion disease, Prnp heterozygosity approximately doubles the survival time (Bueler et al., 1994). Even if PrP^{C} levels were suppressed in prion-infected mice only when early spongiform degeneration or cognitive and neurophysiological prion disease symptoms were beginning to manifest, a partially rescue of these phenotypes could be observed (Mallucci et al., 2003; Mallucci et al., 2007). Consequently, any method that can reduce steady-state levels of PrP^{C} may have merit for the treatment of prion diseases and may ameliorate toxicity in Alzheimer's disease.

Because PrP^{C} is a known cause of prion diseases manifestation (Sailer et al., 1994), reducing its expression is widely considered one of the most promising avenues for their treatment. In contrast to approaches targeting the disease-associated scrapie conformer PrP^{Sc}, the ability to suppress the expression of PrP^{C} should confer protection in a manner that is prion strain-independent. There is robust evidence that reduced PrP^{C} levels due to heterozygous prion gene allele disruption does not cause major functional deficits in mice (Bueler et al., 1994) or humans (Minikel et al., 2016). Finally, the length of the symptom-free prion disease incubation period correlates inversely with the abundance of PrP^{C} (Mays et al., 2014; Minikel et al., 2020), an observation that can be conceptualized on the basis of PrP^{C} not only representing the substrate for conversion into PrP^{Sc} but also for being related to cellular toxicity (Brandner et al., 1996). Even if prion disease symptoms are already manifest, the lowering of PrP^{C} levels may partially reverse both the spongiform degeneration (Mallucci et al., 2003) and the neurophysiological dysfunction that contributes to the cognitive decline (Mallucci et al., 2007).

It is conceivable that the molecular interactions of PrP with other nearby factors play a role in how PrP^{C} contributes to prion diseases. Therefore, considerable efforts have been invested in both the characterization of the microdomain in which PrP^{C} resides and the identification of its interactions. Although no shortage of PrP^{C} binders were described, this body of literature is somewhat fragmented and inconsistent (Watts, Bourkas & Arshad, 2018). A side-by-side analysis of proteins that can be in vivo crosslinked to PrP^{C} in four distinct mouse cell models using identical workflows corroborated the notion that the molecular environment of PrP^{C} can vary from cell model to cell model (Ghodrati et al., 2018). This analysis placed emphasis on the previously reported interaction of PrP^{C} with the neural cell adhesion molecule 1 (Ncam1) (Schmitt-Ulms et al., 2001; Santuccione et al., 2005; Mehrabian, Hildebrandt & Schmitt-Ulms, 2016) by revealing it to be the only protein PrP^{C} could readily be crosslinked to in all four models tested. This analysis also uncovered a shared functional specialization of the molecular environment of PrP^{C} in all four models, namely its close proximity to cellular factors known to modulate the activity of Tgfb1 and integrin sister signaling complexes. The analysis further revealed several other molecules in PrP^{C}-directed co-immunoprecipitates such as Na,K-ATPases (NKAs), which were observed in proximity to PrP^{C} in three out of the four cell models tested (Ghodrati et al., 2018)

To date, attempts to identify PrP^{C} lowering drugs through screens of compound libraries have largely failed, with some of the best lead compounds either requiring relatively high concentrations to exert their effect or lacking favorable ADME characteristics, including the ability to pass the blood brain barrier (Silber et al., 2014; Karapetyan et al., 2013). Recent results from a study that targeted the stability of PrP^{C} transcripts by treating prion-infected mice with antisense oligonucleotides (ASOs) provided elegant proof-of-principle validation of the premise that lowering steady-state PrP^{C} levels can extend survival of prion-infected mice in a dose-dependent manner (Minikel et al., 2020). Robust data for the use of ASOs to address human brain disease exist in the form of advances in the treatment of spinal muscular atrophy (Finkel et al., 2017). Perhaps more relevant, the treatment of cynomolgus monkeys with ASOs designed to induce the destruction of transcripts coding for the tau protein led to approximately 50% reductions in tau protein levels in several brain structures (DeVos et al., 2017).

Challenges associated with adapting this approach for the treatment of human prion diseases relate to the need to inject ASOs periodically through the intrathecal route because mRNA levels are shown to recover two months following bolus injections (Mazur et al., 2019), and the observation of limited ASO delivery to deep brain structures, a caveat that is exacerbated in human adults due to the relatively large brain size (Bennett, 2019). Thus, to date, no treatment exists that can effectively reduce human brain PrP^{C} levels by targeting the expression or stability of this protein directly.

Cardiac glycosides (CGs) are a class of compounds also known as cardiotonic steroids. CGs have a long history of use in the clinic for the treatment of heart disease (Yatime et al., 2009; Greeff, 1981; Schatzmann, 1953). More recently, compounds from this class have been considered for other uses, including the treatment of cancers (Khatri et al., 2019; Mijatovic, Dufrasne & Kiss, 2012; Menger et al., 2013). In addition, the notion to widen the use of CGs to the treatment of stroke and neurodegenerative diseases has had some traction (Wang et al., 2006). To date, brain indications of CGs are limited by the relative narrow therapeutic window and poor blood-brain barrier (BBB) penetration or brain retention of members of this compound class (Kuhlmann, Erdmann & Rietbrock, 1979; Dutta, Marks & Schoener, 1977). Much attention in this regard has been paid to a CG known as oleandrin, which can be derived from the ornamental shrub Nerium oleander (Elmaci et al., 2018; Dunn et al., 2011) and has shown to accumulate to relatively high level in brain tissue (Flasch & Heinz, 1976; Ni et al., 2002). However, oleandrin has recently been shown to exhibit inadvertent cardiotoxicity that exceeded the toxicity of other CGs and may limit its use (Botelho et al., 2020).

U.S. Pat. No. 3,898,331 (Peterson 1975) discloses oleandrin derivative, 4′-dehydro-oleandrin. A synthesis of 4′-dehydro-oleandrin is reported in Chen et al., 2016 as well as its toxicity in HeLa cells.

### SUMMARY

The present application includes a method for treating and/or preventing prion diseases and/or brain diseases, disorders or conditions that benefit from reduced levels of the cellular prion protein (PrP^{C}) and/or altered levels of NKA alpha subunits comprising administering an effective amount of one or more agents that alter levels of one or more NKA alpha subunits, in a subject in need thereof, wherein each NKA alpha subunit with altered levels is a different paralog.

In some embodiments, the one or more agents that alter levels of one or more NKA alpha subunits, are agents that bind to the CG binding domain of the one or more NKA alpha subunits for which levels are decreased.

In some embodiments, the one or more agents that alter levels of one or more NKA alpha subunits are cardiac glycosides, other than oleandrin and neriifolin.

In some embodiments, the one or more agents that decrease levels of one or more NKA alpha subunits are one or more compounds of Formula I, and/or a pharmaceutically acceptable salt and/or solvate thereof:

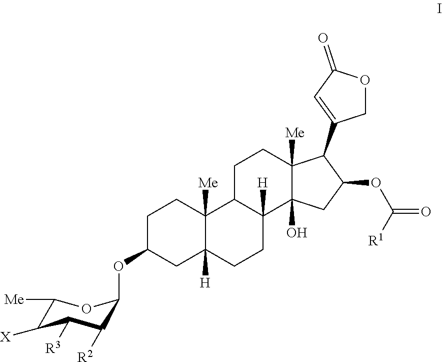

wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH, NH₂, NHC₁₋₄alkyl, and N(C₁₋₄alkyl)₂.

In some embodiments, the one or more agents alter levels of one or more NKA alpha subunit paralogs selected from ATP1A1, ATP1A2 and/or ATP1A3. Therefore, in some embodiments, the conditions associated with altered levels of NKA alpha subunits are brain diseases, disorders or conditions associated with altered ATP1A1, ATP1A2 and/or ATP1A3 levels.

In some embodiments, the brain diseases, disorders or conditions associated with altered ATP1A1, ATP1A2 and/or ATP1A3 levels are selected from rapid-onset dystonia parkinsonism, hemiplegia, autosomal dominant cone-rod dystrophy, Angelman's syndrome, SOD-1 forms of amyotrophic lateral sclerosis and/or forms of ataxia, epilepsy and/or mania.

In some embodiments, the one or more agents that alter levels of one or more NKA alpha subunits decrease levels of one or more NKA alpha subunit paralogs selected from ATP1A1 and/or ATP1A2 and increase levels of ATP1A3. Therefore, in some embodiments, the conditions associated with altered levels of NKA alpha subunits are brain diseases, disorders or conditions associated with increased levels of ATP1A1 and/or ATP1A2, and decreased levels of ATP1A3.

In some embodiments, the prion disease is referred to as a transmissible spongiform encephalopathy (TSE), the alternative and older term for this group of brain disorders. In some embodiments, the prion disease (or TSE), is selected from scrapie in sheep, chronic wasting disease in deer, elk and moose, bovine spongiform encephalopathy in cattle, and Creutzfeldt-Jakob disease, Gerstmann-Sträussler-Scheinker syndrome and fatal familial insomnia in humans.

In some embodiments, the brain diseases, disorders or conditions that benefit from reduced levels of the cellular prion protein (PrP^{C}) are selected from Alzheimer's disease, Parkinson's disease and/or amyotrophic lateral sclerosis.

In some embodiments, the diseases, disorders or conditions are treated or prevented in the methods and uses of the present application by decreasing PrP^{C} levels. In some embodiments, the decreasing of PrP^{C} levels is by increasing PrP^{C} degradation. In some embodiments, the PrP^{C} degradation is by endolysosomal degradation. In some embodiments, the PrP^{C} degradation is by endolysosomal cathepsin-dependent degradation.

In some embodiments, the present application also includes a compound of Formula I-A or a pharmaceutically acceptable salt and/or solvate thereof:

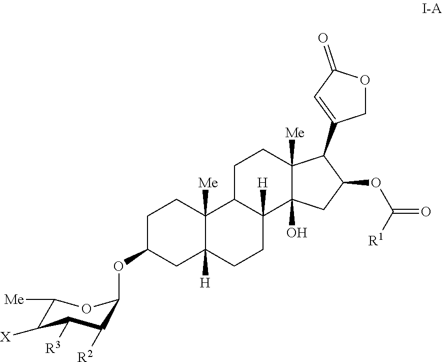

wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH and NH₂;
provided when X is ═O, R² is H and R³ is OCH₃, then R¹ is not CH₃ or CF₃.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF DRAWINGS

The embodiments of the application will now be described in greater detail with reference to the attached drawings in which:

FIG. 1 shows the study overview for Example 1. A shows the workflow of brain PrP interactome analysis. B shows CRISPR/Cas9-based knockout of PrP in human co-culture model of differentiated neurons and glia cells. C shows the validation of top-listed PrP interactor.

FIG. 2 shows the evidence for selective PrP co-enrichment of NKAs in mouse brain. A shows quantitative mass spectrometry sample processing workflow. B to D show side-by-side box plots depicting relative peptide enrichment levels in tryptic digests of PrP brain interactome eluates. B shows selective enrichment of PrP-derived peptides in wild-type eluates (not in PrP ko eluates). C shows exemplary PrP co-enrichment of the NKA alpha-1 subunit ATP1A1 (note that other NKA subunits also were observed co-enriched). D shows the myelin basic protein (MBP) was observed at similar abundance levels in all samples, including PrP interactome eluates from PrP ko cells, consistent with the interpretation that it co-purified non-specifically with the affinity matrix.

FIG. 3 shows the validation of NKA binding to PrP. A shows western blot-based validation of co-immunoprecipitation of NKA subunits with PrP. B shows evidence of partial cellular co-localization of PrP^{C} and ATP1A1. C shows the chemical structure of the NKA inhibitor ouabain. D shows parallel ouabain concentration-dependent effects on NKA and PrP^{C} levels. Exposure of differentiated ReN cells to ouabain at concentrations up to 50 nM causes a biphasic effect on PrP^{C} and ATP1A1 levels, with concentrations up to 30 nM leading to an ouabain concentration-dependent reduction in steady-state levels of both proteins, and exposure to 50 nM provoking a reproducible slowing of PrP^{C} during SDS-PAGE separation that parallels an increase in ATP1A1 levels.

FIG. 4 shows the PrP deficiency or prion infection alter ⁸⁶Rb⁺ uptake activity of NKA. A shows the design of gRNA targeting PRNP coding sequence. B shows the CRISPR/Cas9-based knockout of PrP in ReN cells. C shows the validation of PrP knockout in ReN cells. Note that PrP deficiency has no effect on steady-state ATP1A1 levels in this model. D shows that PrP knockout diminishes ⁸⁶Rb⁺ uptake in ReN cells. E shows that increased steady-state ATP1A1 levels in RML-infected Neuro2a cells. D shows that similar to PrP knockout, RML-infection of Neuo2a cells compromises ⁸⁶Rb⁺ uptake, albeit to a lesser extent.

FIG. 5 shows ReN cells respond to PrP-deficiency or CG exposure with an increase in the expression of a 60 kDa Coomassie-stained signal, originating from 5′ ectonucleotidase. A shows the observation of Coomassie-stained protein band of 60 kDa that is conspicuously increased in the presence of cardiotonic glycosides. B shows MS/MS spectrum documenting identification of CD73 as the protein underlying this band. C shows spectral count comparison of MS-based NT5E identification from Western blot bands observed in the absence or presence of ouabain. D shows Western blot validation of increased CD73 protein levels upon prolonged exposure to low levels of ouabain. E shows CRISPR/Cas9-mediated knockout of PrP mimics low levels of ouabain in its effect on steady-state CD73 levels. To minimize potential clonal effects, the increase in band intensity is seen in pools of CRISPR/Cas9 gene engineered PrP knockout cells using 3 different gRNAs, that differ in the percentages of cells depleted for PrP expression. Note that the CD73 increase correlates inversely with the degree of PrP knockout (i.e., the percentage of cells exhibiting the knockout).

FIG. 6 show the prolonged exposure of ReN cells to CG or prion infection causes cleavage of GFAP. A shows a Western blot documenting an increase in the intensity of GFAP antibody-reactive signals of 40-48 kDa in aged prion-infected mice that is not observed in vehicle-infected littermates. B shows a Western blot documenting similar appearance of GFAP reactive bands in differentiated ReN cells exposed to low nanomolar concentrations of ouabain. C shows Calpain I inhibitor blocks ouabain-dependent formation of GFAP antibody reactive bands of 40-48 kDa. D shows side-by-side western blot analysis of differentiated ReN cells, treated in the presence or absence of a calpain I inhibitor (A6185) next to brain lysates of RML-infected mice.

FIG. 7 shows the steady-state levels of isoforms of PrP^{C} and NKA alpha-subunits are individually controlled by extracellular CGs and intracellular Ca²⁺ ions. Blocking the reverse mode activity of the Na,Ca-exchanger by 3-day exposure of ReN VM cells to YM24476 increased the steady-state levels of PrP^{C} and ATP1A3, had little effect on ATP1A1 levels, and caused a reduction of ATP1A2. Addition of the calpain inhibitor A6185 diminished PrP^{C} and ATP1A2 levels but had no apparent effect on ATP1A1 and ATP1A3 levels. Exposure to 20 nM ouabain caused a reduction in the steady-state levels of PrP^{C}, ATP1A1 and ATP1A2—but not ATP1A3—in a manner that cannot be reversed by concomitant incubation with YM24476. In contrast, co-incubation of cells with ouabain and A6185 rescued the reduction in ATP1A1 levels and caused the appearance of signals detected with the PrP^{C}- and ATP1A2-directed antibodies that migrated with different apparent molecular weights than the dominant signals for these proteins observed in untreated cells. Finally, the intensity of relatively fast migrating signals detected with a GFAP-directed antibody increase under conditions that favor intracellular calcium-dependent calpain activity.

FIG. 8 shows the prolonged non-toxic CG exposure of human ReN cells causes concentration-dependent reduction in NKA α subunit and PrP^{C} levels. A shows workflow of in vitro ReN cell-based CG exposure analysis. B shows exposure of differentiated ReN cells to ouabain at low nanomolar concentrations (up to 20 nM) causes a concentration-dependent reduction in PrP^{C} and ATP1A1 levels that is already apparent at 3 nM ouabain levels. Levels of actin or other proteins in the cell visualized by Coomassie staining are not similarly affected by ouabain. C shows a Western blot documenting that extended exposure of differentiated neurons to low levels of digoxin (up to 20 nM) caused a dose-dependent reduction in PrP^{C} levels—and to a lesser extent CD109—but did not affect the levels of a majority of other cellular proteins, as evidenced by Coomassie staining. D are graphs depicting quantitations of steady-state levels of PrP^{C} and CD109 in the presence of non-toxic low nanomolar levels of digoxin. E shows the effects of various concentrations of digoxin (0-500 nM) on cell viability (black squares), cellular ATP content (red triangles) and intracellular Ca2+ concentration ([Ca2+]i, green circles). Toxic concentrations of digoxin are shaded in grey.

FIG. 9 shows that the CG-dependent reduction of PrP^{C} levels relies predominantly on endolysosomal degradation pathway. A and B show that inhibition of calpain proteases with calpastatin or calpain inhibitor did not rescue the reduction of PrP^{C} levels that is observed in ouabain-treated cells. C and D show that proteasomal inhibition with MG132 or lactacystin did not rescue the reduction of PrP^{C} levels that is observed in ouabain-treated cells. E and F show that, in contrast, inhibition of endolysosomal degradation pathways with bafilomycin A1 or E64D blocked the ouabain-dependent PrP^{C} reduction. Quantitative graphs in this figure depict the means of signal intensities and their standard deviations. Asterisks reflect significance thresholds with one asterisk indicating p<0.05 and two asterisks are depicted when p<0.005.

FIG. 10 shows the favorable characteristics of oleandrin as a candidate CG for the Na/K ATPase-mediated reduction of PrP^{C} levels. A and B show the comparison of (A) ouabain, a cardiotonic steroid sourced from the Strophanthus gratus plant native to eastern Africa, and (B) oleandrin, found in Nerium oleander, an ornamental shrub of uncertain origin. A lack of hydroxyl groups in the steroid core and the presence of additional methyl groups that are absent in ouabain account for the more hydrophobic characteristics of oleandrin. C depicts effects of various concentrations of oleandrin (0-10 nM) on cell viability (black squares), cellular ATP content (red triangles) and intracellular Ca²⁺ concentration ([Ca²⁺]ᵢ, green circles). Toxic concentrations of oleandrin are shaded in grey. D shows that tenfold lower levels of oleandrin than ouabain cause a similar reduction in steady-state PrP^{C} levels during 7-day treatment of differentiated ReN VM cells. E shows graphs comparing the means of western blot signal intensities of ATP1A1 and PrP^{C} levels and their standard deviations in 2 nM oleandrin- versus 20 nM ouabain-treated ReN VM cells. The number of asterisks shown reflect significance thresholds as follows: one: p<0.05; two: p<0.005; three: p<0.0005.

FIG. 11 shows CG-dependent reduction of PrP^{C} levels depends on CG-ATP1A1 ligand-receptor engagement. A shows predicted oleandrin binding pose modeled in ATP1A1 structure reported in PDB entry 4HYT. B shows genetic sequencing results confirm amino acid substitutions Q118R and N129D, two changes required to render the human ATP1A1 resistant to CGs, in a ReN VM clone. C shows a comparison of ATP1A1 levels (top panel) and PrP^{C} levels (bottom panel) in response to oleandrin treatment in ReN VM cells that express either the sensitive wild-type or the CRISPR-Cas9-engineered resistant form of ATP1A1. In wild-type cells levels of ATP1A1 and PrP^{C} decreased as oleandrin concentration increased from 0 to 1 nM. When oleandrin was administered at a concentration of 2 nM, levels of ATP1A1 and PrP^{C} rebounded and toxicity was observed (grey shaded box). In contrast, levels of ATP1A1 and PrP^{C} both remained stable at all tested oleandrin concentrations in cells expressing the resistant form of ATP1A1, and no toxicity was observed. Coomassie staining documents that protein loading was adjusted across lanes. D are graphs depicting quantitations of steady-state levels of ATP1A1 in the presence of oleandrin concentrations up to 3 nM in CG sensitive wildtype and resistant ReN VM cells.

FIG. 12 shows a study design overview. A shows in silico identification of a CG with favorable predicted docking characteristics and BBB barrier penetrance. B shows synthesis and characterization of short-listed CG. C shows the assessment of BBB penetrance and distribution in relevant tissue. D shows the in vitro characterization of potency and mechanism of action for PrP^{C} reduction of lead CG in cell based assay.

FIG. 13 shows the in silico prediction of CG binding pose within relevant human NKA alpha subunits and summary of CG design considerations. A shows electron densities of CG binding pose in PDB entry 4HYT (Laursen et al., 2013). B shows structural alignment of ouabain (brown) and bufalin (green) within the CG binding pocket of two well-resolved X-ray crystallography models (PDB entries 4HYT and 4RES (Laursen et al., 2015)) of NKAs. C shows the sequence identities between porcine and human NKA α subunits known to be expressed in the brain. D shows the comparison of observed porcine and predicted human amino acids lining the CG binding pocket. E shows the generalized Born binding pose and predicted free binding energies for oleandrin within human ATP1A3 model. F shows the surface area-optimized binding pose and predicted free binding energies for oleandrin within human ATP1A3 model. G shows the spatial alignment of observed and predicted binding poses of ouabain and oleandrin in experimentally deduced ATP1A1 and predicted surface-area optimized ATP1A3 models, respectively. H shows points of consideration for the design of potent CGs with optimized brain bioavailability.

FIG. 14 shows the in silico filtering of CG derivatives accessible through chemical derivatization on the basis of their predicted docking scores and Rankovic multi-parameter optimized (MPO, version 2) BBB penetrance scores. A shows the design of CG scaffolds and chemically accessible modifications evaluated, giving rise to a combinatorial total of 270 CGs considered. B shows the workflow for the in silico assessment of candidates. The assignment of protonation states and partial charges increased total CG ligands evaluated to 330, a number that further increased to 850 once alternative binding poses, predominantly caused by freely rotating bonds were considered. The elimination of binding poses with unfavorable internal energy or deviation from the hypothetical oleandrin binding pose led to a 146 CGs that passed these filters. C shows an exemplary chart depicting results from the evaluation of Scaffold 1 CG derivatives. The shading scheme reflects Rankovic MPO.v2 scores (Rankovic, 2017) (a high score, represented by a medium grey squares, indicates high predicted BBB penetrance) and the size of squares reflects docking strength (a low docking score, represented by a large square, indicates strong binding). The absence of a square indicates that no binding pose that passed filter criteria (see above) was found. D is a summary chart depicting results from the evaluation of Rankovic MPO.v2 scores and docking scores for all five scaffolds. Note that CGs derived from Scaffold 4 had similar docking scores as derivatives from other scaffolds but excelled on the basis of their high predicted BBB penetrance.

FIG. 15 shows features of chemically accessible Scaffold 4 derivatives of interest. A shows the chemical structure and reference Rankovic MPO.v2 and docking scores of oleandrin. B shows oleandrin derivatives of interest. Starting with oleandrin, several compounds with high predicted Rankovic MPO.v2 scores can be accessed through C4′-dehydro-oleandrin (here termed exemplary compound I-3) or C4′-amine-oleandrin (Chen et al., 2016). C shows the chemical structures of compounds listed in Bwith their respective Rankovic MPO.v2 and Glide scores. Note that structures of exemplary compounds I-2, I-4, I-6 and I-8 are not depicted. D to F show close-up models providing plausible explanations for predicted increases of Glide docking scores (residue numbers as in PDB 4HYT). D shows a salt bridge between the C4′ amine and the side-chain of glutamic acid residue 312. E shows hydrogen bonding opportunity for the C3′ isopropyl group with the side chain of aspartic acid residue 116. F shows the good fit of C16 trifluoro-acetoxy group within hydrophobic binding pocket shaped from side-chain atoms of phenylalanine 783 and isoleucine 800 residues lining the CG binding pocket.

FIG. 16 shows graphs showing the improved brain bioavailability of exemplary compound I-3 relative to comparative compound, oleandrin. A shows the time-series comparing tissue bioavailability of exemplary compound I-3 and comparative compound, oleandrin following acute subcutaneous injection of tritium labeled compounds. B shows the reduced risk of inadvertent cardiotonic effects of exemplary compound I-3 due to high brain-to-heart ratio following acute subcutaneous injection of tritium-labeled compounds into cohorts of five mice.

FIG. 17 shows the potency of exemplary compound I-3 in PrP^{C} reduction assay. A shows the side-by-side comparison of potency of exemplary compound I-3 and oleandrin in regard to their ability to reduce PrP^{C} levels in ReN VM cell model following 7 day of treatment. B shows the exemplary compound I-3 concentration-dependent reduction in steady-state ATP1A1, ATP1A2 and PrP^{C} protein levels that is paralleled by an increase in ATP1A3 levels. Total protein was adjusted prior to gel loading as evidenced by the Coomassie stain. C shows the biological replicates of subset of exemplary compound I-3 concentrations tested in Panel B. D shows the quantitation of western blot signal intensities of ATP1A1 and PrP^{C}.

FIG. 18 shows that the engagement of exemplary compound I-3 with ATP1A1 is involved in the reduction in steady-state levels of ATP1A2 and PrP^{C} in a manner that does not shift the relative abundance of neuronal or astrocytic markers. A shows that exemplary compound I-3 causes the expected reduction in the steady-state levels of ATP1A1 and ATP1A2 in wild-type ReN VM cells but not in ReN VM r/r cells rendered resistant to CG binding through a double point mutation in the ATP1A1 gene (r/r). B shows that the reduction in steady-state PrP^{C} levels also relies on exemplary compound I-3 binding to ATP1A1, as opposed to some unspecified other effect of the CG on the cell. C shows that despite the decrease in ATP1A2 levels, no reduction in the steady-state levels of astrocytic GFAP is observed.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

The term “compound(s) of the application” or “compound(s) of the present application” and the like as used herein refers to a compound of Formula I and I-A or pharmaceutically acceptable salts and/or solvates thereof.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “fluoroalkyl” refers to an alkyl wherein one or more, including all, available hydrogens in a referenced group have been substituted with fluoro.

The term “available”, as in “available hydrogen atoms” or “available atoms” refers to atoms that would be known to a person skilled in the art to be capable of replacement by a substituent.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice. A suitable solvent is physiologically tolerable at the dosage administered.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

The term “to treat”, treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of one or more of the compounds of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with prion diseases and/or other brain diseases, disorders or conditions that benefit from reduced levels of the cellular prion protein (PrP^{C}), or manifesting a symptom associated with prion diseases and/or other brain diseases, disorders or conditions that benefit from reduced levels of the cellular prion protein (PrP^{C}).

As used herein, the term “effective amount” or “therapeutically effective amount” means an amount of an agent, compound, one or more agents or one or more compounds, that is effective, at dosages and for periods of time necessary to achieve the desired result. For example, in the context of treating prion diseases and/or other brain diseases, disorders or conditions that benefit from reduced levels of the cellular prion protein (PrP^{C}), an effective amount is an amount that, for example, decreases levels of cellular prior protein (PrP^{C}) compared to levels of cellular prion protein (PrP^{C}) without administration of the agent, compound, one or more agents or one or more compounds. Effective amounts may vary according to factors such as the disease state, age, sex and/or weight of the subject. The amount of a given agent or compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

The term “brain disease, disorder or condition that benefits from reduced levels of the cellular prion protein (PrP^{C})” as used herein refers to any disease, disorder or condition that is directly or indirectly caused by, or has as part of its etiology, the cellular prion protein (PrP^{C})in a subject's brain. Such diseases, disorders or conditions will also be any disease, disorder or condition that benefits from decreasing PrP^{C}levels.

The term “increased” or “increasing” in reference to levels of a protein or other entity, including PrP^{C}, ATP1A1, ATP1A2 and/or ATP1A3, as used herein means any detectable increase in the level of the protein in a cell or a subject compared to the necessary, normal or desirable level of the protein in the cell or the subject and/or any detectable increase in the level of the protein in a cell or a subject in the presence of a treatment compared to otherwise the same conditions except in the absence of the treatment.

The term “decreasing”, “decreased”, “reduced” or “reducing” in reference to levels of a protein or other entity, including PrP^{C}, ATP1A1, ATP1A2 and/or ATP1A3, as used herein means any detectable decrease or reduction in the level of the protein in a cell or a subject compared to the necessary, normal or desirable level of the protein in the cell or the subject and/or any detectable decrease or reduction in the level of the protein in a cell or a subject in the presence of a treatment compared to otherwise the same conditions except in the absence of the treatment.

The term “alter” or “altered” in reference to levels of a protein or other entity, as in altered levels of NKA alpha subunits, as used herein refers to any detectable increase or decrease in the level of the protein in a cell or a subject compared to the necessary, normal or desirable level of the protein in the cell or the subject and/or any detectable increase or decrease in the level of the protein in a cell or a subject in the presence of a treatment compared to otherwise the same conditions except in the absence of the treatment.

The term “levels” as in levels of PrP^{C}, ATP1A1, ATP1A2, and/or ATP1A3, as used herein means the amount of these proteins and/or the transcripts that code for them in a cell or a subject as determined by northern blotting, PCR, quantitative PCR, western blotting, ELISA, or immunocytochemistry.

By “downregulate” or “downregulation” it is meant any means for decreasing the level and/or activity of a protein, including degradation.

By “upregulate” or “upregulation” it is meant any means for increasing the level and/or activity of a protein.

The term “prion diseases” as used herein refers to diseases directly or indirectly caused by, or has as part of their etiology, PrP^{C}. These diseases are sometimes still referred to by the older term, transmissible spongiform encephalopathies (TSEs).

The term “ATP1A1” as used herein refers to ATPase, Na+/K+ transporting, alpha 1 polypeptide. Species-specific sequences for this protein can be retrieved from: https://www.uniprot.org/uniprot/?query=atp1a1&sort=score.

The term “ATP1A2” as used herein refers to ATPase, Na+/K+ transporting, alpha 2 polypeptide. Species-specific sequences for this protein can be retrieved from: https://www.uniprot.org/uniprot/?query=atp1a2&sort=score.

The term “ATP1A3” as used herein refers to ATPase, Na+/K+ transporting, alpha 3 polypeptide. Species-specific sequences for this protein can be retrieved from: https://www.uniprot.org/uniprot/?query=atp1a3&sort=score.

The term “PrP^{C}” or “cellular prion protein” refers to the naturally occurring or normal form of prion protein. Species-specific sequences for this protein can be retrieved from: https://www.uniprot.org/uniprot/?query=prnp&sort=score.

The term “cardiac glycoside” or “cardiotonic steroid” as used herein refers to a class of organic compounds that comprises a steroid molecule, a sugar molecule (glycoside) and an “R” group as shown in the following general structure:

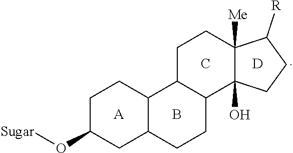

Other functional groups can be attached to the steroid nucleus and the R group can vary and is often a lactone moiety. This class of organic compounds are known to increase the output force of the heart and decrease its rate of contractions by acting on the cellular Na,K-ATPase pump.

The term “oleandrin” as used herein refers to a compound having the chemical name acetic acid [(3S,5R,10S,13R,14S,16S,17′R)-14-hydroxy-3-[[(2R,4S,5S,6S)-5-hydroxy-4-methoxy-6-methyl-2-tetrahydropyranyl]oxy]-10,13-dimethyl-17-(5-oxo-2H-furan-3-yl)-1,2,3,4,5,6,7,8,9,11,12,15,16,17-tetradecahydrocyclopenta[a]phenanthren-16-yl] ester and having the chemical formula:

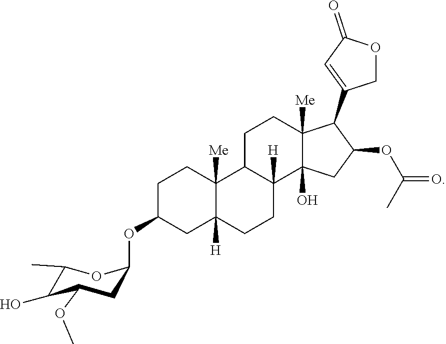

The term “administered” as used herein means administration of a therapeutically effective amount of one or more compounds or a pharmaceutically acceptable salt and/or solvate thereof or compositions comprising a compound or a pharmaceutically acceptable salt and/or solvate thereof to a cell, tissue, organ or subject.

The term “subject” as used herein includes all members of the animal kingdom, including mammals, and suitably refers to humans. Thus, the methods and uses of the present application are applicable to both human therapy and veterinary applications.

The term “pharmaceutical composition” as used herein refers to a composition of matter for pharmaceutical use.

The term “pharmaceutically acceptable” means compatible with the treatment of subjects.

The term “paralog” as used herein refers to genes within a species that were derived from the same ancestral gene.

### II. Methods and Uses of the Application

The Applicants have found that Na,K-ATPases (NKAs) are proximate to cellular prion protein (PrP^{C}) in the cell membranes of mouse brains and that this spatial proximity of PrP^{C} to NKAs translates into a direct influence of PrP^{C} on the ion transport driven by the NKAs. The Applicants have also found that the binding of cardiac glycosides (CGs) to NKAs surprisingly causes not only the internalization and degradation of NKAs but also of other NKA-associated molecules, including PrP^{C}. This PrP^{C} degradation was shown in human co-cultures of neurons and astrocytes to be dependent on a CG ligand-NKA alpha subunit receptor engagement and therefore levels of the CG ligand-bound NKA alpha subunit decreased together with CG ligand levels in the cell culture medium. Further, as levels of the NKA alpha subunit bound to the CG ligand decreased, levels of other NKA alpha subunits with lower binding affinity to the CG ligand were increased together with CG ligand levels in the cell culture medium.

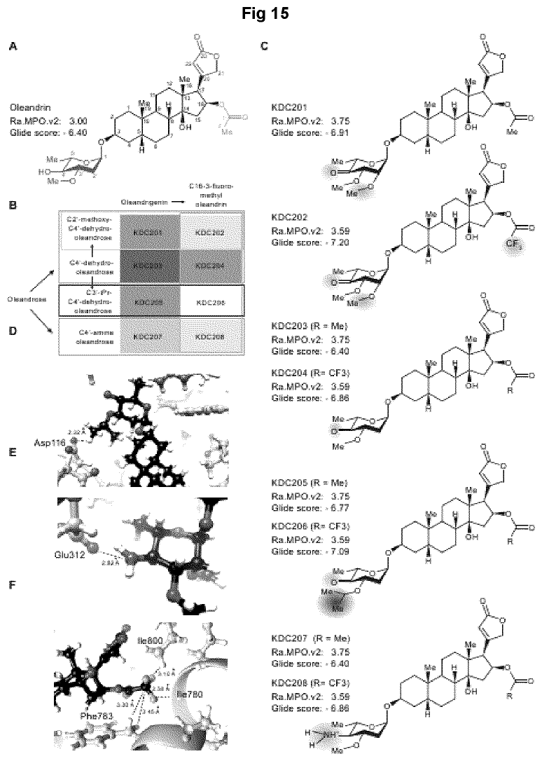

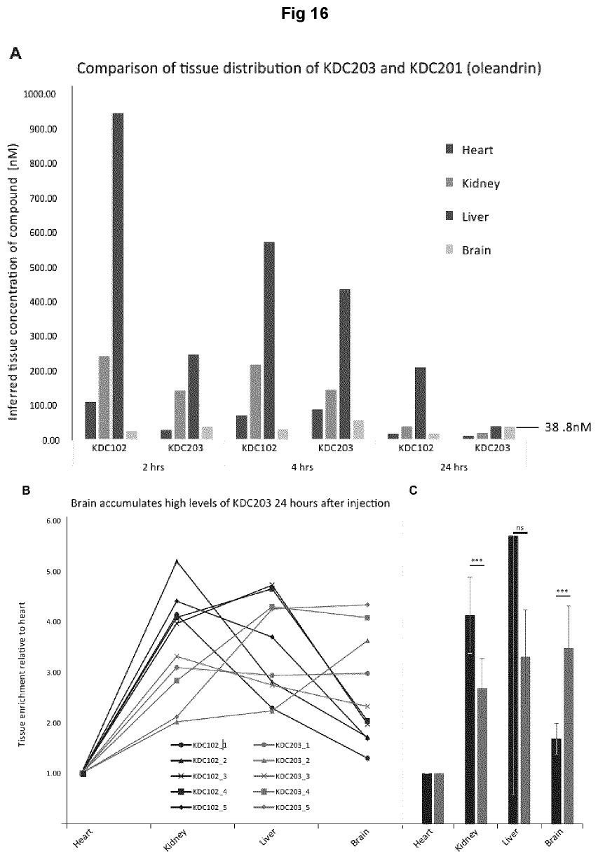

Oleandrin is a known CG that has relatively high brain bioavailability when compared to other CGs. However, a relatively low Rankovic MPO.v2 score of 3.0 (FIG. 15 A) of oleandrin suggests its brain penetrance can be further improved. Moreover, the similar levels of oleandrin in heart and brain, 24 hours after acute injection (FIG. 16), are expected to limit its use for brain applications due to the manifestation of inadvertent cardiotonic affects. Starting from oleandrin and using in silico modeling of CG binding poses within human NKA folds, CG structure-activity relationship (SAR) data, and predicted blood-brain barrier (BBB) penetrance scores, the Applicants have identified oleandrin derivatives with improved pharmacological properties. In some embodiments, the Applicants have shown that the oleandrin derivative, C4′-dehydro-oleandrin, demonstrates improved brain bioavailability combined with low heart levels and also suppresses PrP^{C} levels in human co-cultures of neurons and astrocytes. Further, the Applicants have surprisingly found that the PrP^{C} level reduction achieved by the oleandrin derivative C4′-dehydro-oleandrin leads to a reduction in ATP1A1 and ATP1A2 levels but an increase in ATP1A3 levels.

Accordingly, the present application includes a method for treating and/or preventing prion diseases and/or brain diseases, disorders or conditions that benefit from reduced levels of the cellular prion protein (PrP^{C}) and/or altered levels of NKA alpha subunits comprising administering and effective amount of one or more agents that alter levels of one or more NKA alpha subunits, in a subject in need thereof, wherein each NKA alpha subunit with altered levels is a different paralog.

The application also includes a use of one or more agents that alter levels of one or more NKA alpha subunits for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog. The application also includes a use of one or more agents that alter levels of one or more NKA alpha subunits for preparation of a medicament for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog. The application also includes one or more agents that alter levels of one or more NKA alpha subunits for use to treat and/or prevent prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog.

In some embodiments, the one or more agents that alter levels of one or more NKA alpha subunits, are agents that bind to the cardiac glycoside binding domain of the one or more NKA alpha subunits for which levels are decreased.

In some embodiments, the one or more agents that alter levels of one or more NKA alpha subunits are cardiac glycosides, other than oleandrin and neriifolin.

In some embodiments, the one or more agents that alter levels of one or more NKA alpha subunits, are one or more compounds of Formula I, and/or a pharmaceutically acceptable salt and/or solvate thereof:

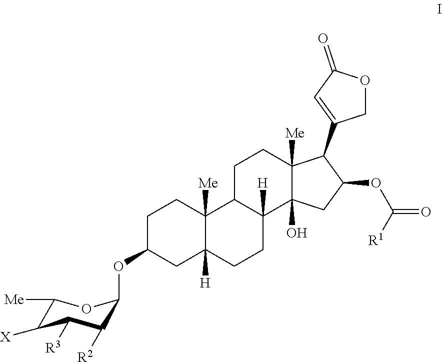

wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH, NH₂, NHC₁₋₄alkyl, and N(C₁₋₄alkyl)₂.

In some embodiments, R¹ is selected from C₁₋₃alkyl and C₁₋₃fluoroalkyl. In some embodiments, R¹ is selected from CH₃, CF₃, CF₂H, CFH₂, CH₂CH₃, CF₂CF₃, CH₂CF₂H, CH₂CF₂H, CH(CH₃)₂, CF(CF₃)₂, C(CF₃)₃, and C(CH₃)₂. In some embodiments, R¹ is selected from CH₃, CF₃, CH₂CH₃, CF₂CF₃, CH(CH₃)₂, CF(CF₃)₂, C(CF₃)₃, and C(CH₃)₂. In some embodiments, R¹ is selected from CH₃, CF₃, CH₂CH₃, CF₂CF₃, CF₂H, CH(CH₃)₂ and CF(CF₃)₂. In some embodiments, R¹ is selected from CH₃, CF₃, CH₂CH₃, CF₂CF₃, CH(CH₃)₂ and CF(CF₃)₂. In some embodiments, R¹ is selected from CH₃, CF₃, CH₂CH₃ and CF₂CF₃. In some embodiments, R¹ is selected from CH₃ and CF₃.

In some embodiments, R² and R³ are independently selected from H, OC₁₋₃alkyl and OC₁₋₃fluoroalkyl. In some embodiments, R² and R³ are independently selected from H, OCH₃, OCF₃, OCF₂H, OCFH₂, OCH₂CH₃, OCH₂CF₂H, OCH₂CF₂H, OCF₂CF₃, OCH(CH₃)₂, OCF(CF₃)₂, OC(CF₃)₃, and OC(CH₃)₂. In some embodiments, R² and R³ are independently selected from H, OCH₃, OCF₃, OCH₂CH₃, OCF₂CF₃, OCH(CH₃)₂, OCF(CF₃)₂, OC(CF₃)₃, and OC(CH₃)₂. In some embodiments, R² and R³ are independently selected from H, OCH₃, OCF₃, OCH₂CH₃, OCF₂CF₃, OCH(CH₃)₂ and OCF(CF₃)₂. In some embodiments, R² is selected from H, OCH₃ and OCF₃. In some embodiments, R² is H. In some embodiments, R² is selected from OCH₃ and OCF₃. In some embodiments, R³ is selected from H, OCH₃, OCF₃, OCH(CH₃)₂ and OCF(CF₃)₂. In some embodiments, R³ is selected from OCH₃, OCF₃, OCH(CH₃)₂ and OCF(CF₃)₂. In some embodiments, R³ is selected from OCH₃ and OCH(CH₃)₂. In some embodiments, R² is selected from H, OCH₃ and OCF₃ and R³ is selected from OCH₃, OCF₃, OCH(CH₃)₂ and OCF(CF₃)₂. In some embodiments, R² is H, and R³ is selected from OCH₃, OCF₃, OCH(CH₃)₂ and OCF(CF₃)₂. In some embodiments, R² is H, and R³ is selected from OCH₃, OCF₃ and OCH(CH₃)₂.

In some embodiments, X is selected from ═O, ═NH, NH₂, NHCH₃, and N(CH₃)₂. In some embodiments, X is selected from ═O, NH₂, NHCH₃, and N(CH₃)₂. In some embodiments, X is selected from ═O and ═NH. In some embodiments, X is selected from ═O and NH₂. In some embodiments, X is ═O. In some embodiments, X is NH₂.

In some embodiments, X is NH₂ and the one or more compounds of Formula I are pharmaceutically acceptable salts thereof.

In some embodiments, the one or more compounds of Formula I are selected from the compounds listed below:

| Compound | |
|---|---|
| I.D | Structures |
| I-1 | 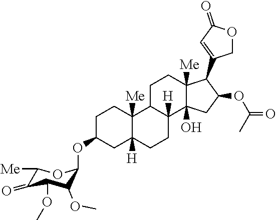 |
| I-2 | 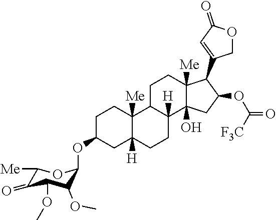 |
| I-3 | 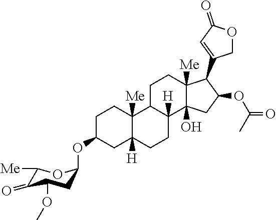 |
| I-4 | 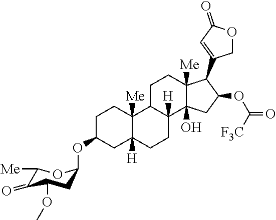 |
| I-5 | 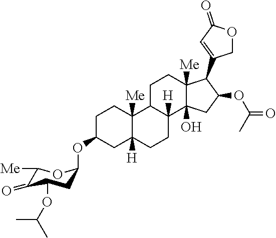 |
| I-6 | 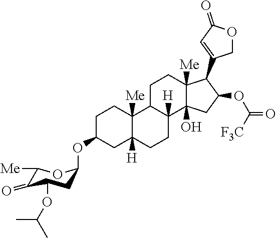 |
| I-7 | 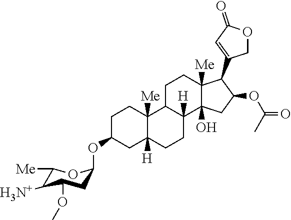 |
| I-8 | 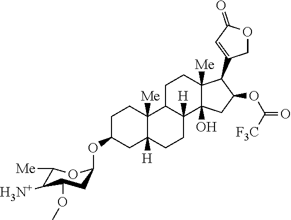 |

and/or pharmaceutically acceptable salts and/or solvates thereof.

In some embodiments the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates include, for example, those made with solvents that are pharmaceutically acceptable. Examples of such solvents include water (resulting solvate is called a hydrate) and ethanol and the like. Suitable solvents are physiologically tolerable at the dosage administered.

In some embodiments, the one or more agents that alter levels of one or more NKA alpha subunits decrease the levels of the one or more NKA alpha subunits that binds the one or more agents most strongly. In some embodiments the one or more agents bind most strongly to two or more different NKA alpha subunit paralogs and the levels of each NKA alpha subunit paralog are decreased.

In some embodiments, the one or more agents that alter levels of one or more NKA alpha subunits increase the levels of the one or more NKA alpha subunits that binds the one or more agents least strongly. In some embodiments the one or more agents bind least strongly to two or more different NKA alpha subunit paralogs and the levels of each NKA alpha subunit paralog are increased.

In some embodiments, the one or more agents alter levels of one or more NKA alpha subunit paralogs selected from ATP1A1, ATP1A2 and/or ATP1A3. Therefore, in some embodiments, the conditions associated with altered levels of NKA alpha subunits are brain diseases, disorders or conditions associated with altered ATP1A1, ATP1A2 and/or ATP1A3 levels.

In some embodiments, the brain diseases, disorders or conditions associated with altered ATP1A1, ATP1A2 and/or ATP1A3 levels are selected from rapid-onset dystonia parkinsonism, hemiplegia, autosomal dominant cone-rod dystrophy, Angelman's syndrome, SOD-1 forms of amyotrophic lateral sclerosis and/or forms of ataxia, epilepsy and/or mania.

In some embodiments, the one or more agents that alter levels of one or more NKA alpha subunits decrease levels of one or more NKA alpha subunit paralogs selected from ATP1A1 and/or ATP1A2 and increase levels of ATP1A3. Therefore, in some embodiments, the conditions associated with altered levels of NKA alpha subunits are brain diseases, disorders or conditions associated with increased levels of ATP1A1 and/or ATP1A2, and decreased levels of ATP1A3.

In some embodiments, the prion disease is referred to as a transmissible spongiform encephalopathy (TSE), the alternative and older term for this group of brain disorders. In some embodiments, the prion disease (or TSE), is selected from scrapie in sheep, chronic wasting disease in deer, elk and moose, bovine spongiform encephalopathy in cattle, and Creutzfeldt-Jakob disease, Gerstmann-Sträussler-Scheinker syndrome and fatal familial insomnia in humans. In some embodiments, the deer is sika deer and/or reindeer.

In some embodiments, the brain diseases, disorders or conditions that benefit from reduced levels of the cellular prion protein (PrP^{C}) are selected from Alzheimer's disease, Parkinson's disease and/or amyotrophic lateral sclerosis.

In some embodiments, the diseases, disorders or conditions are treated or prevented in the methods and uses of the present application by decreasing PrP^{C} levels. In some embodiments, the decreasing of PrP^{C} levels is by increasing PrP^{C} degradation. In some embodiments, the PrP^{C} degradation is by endolysosomal degradation. In some embodiments, the PrP^{C} degradation is by endolysosomal cathepsin-dependent degradation.

In some embodiments, the present application also includes a method for treating and/or preventing prion diseases and/or brain diseases, disorders or conditions that benefit from reduced levels of the cellular prion protein (PrP^{C}) and/or altered levels of NKA alpha subunits comprising administering an effective amount of one or more agents that alter levels of one or more NKA alpha subunits, in combination with an effective amount of another known agent useful for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog.

In some embodiments, the present application also includes a use of one or more agents that alter levels of one or more NKA alpha subunits, in combination with an effective amount of another known agent useful for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog, for treating and/or preventing prion diseases and/or brain diseases, disorders or conditions that benefit from reduced levels of the cellular prion protein (PrP^{C}) and/or altered levels of NKA alpha subunits. In some embodiments, the present application also includes a use of one or more agents that alter levels of one or more NKA alpha subunits, in combination with an effective amount of another known agent useful for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog, for preparation of a medicament for treating and/or preventing prion diseases and/or brain diseases, disorders or conditions that benefit from reduced levels of the cellular prion protein (PrP^{C}) and/or altered levels of NKA alpha subunits. In some embodiments, the present application also includes one or more agents that alter levels of one or more NKA alpha subunits, for use in combination with an effective amount of another known agent useful for treating and/or preventing prion diseases and/or brain diseases, disorders or conditions that benefit from reduced levels of the cellular prion protein (PrP^{C}) and/or altered levels of NKA alpha subunits, wherein each NKA alpha subunit with altered levels is a different paralog, to treat and/or prevent prion diseases and/or brain diseases, disorders or conditions that benefit from reduced levels of the cellular prion protein (PrP^{C}) and/or altered levels of NKA alpha subunits.

In some embodiments, the known agent useful for treating and/or preventing prion diseases and/or brain diseases, disorders or conditions that benefit from reduced levels of the cellular prion protein (PrP^{C}) and/or altered levels of NKA alpha subunits is selected from one or more of flupirtine, quinacrine, pentosan polysulfate (PPS), doxycycline, CompB, IND24, IND18, Anle138b, Congo red, Fe(III)TMPyP, guanabenz, resveratrol, curcumin, melatonin and gallic acid. In some embodiments, known agent useful for treating and/or preventing prion diseases and/or brain diseases, disorders or conditions that benefit from reduced levels of the cellular prion protein (PrP^{C}) and/or altered levels of NKA alpha subunits is an immunotherapeutic. In some embodiments, the immunotherapeutic is an anti-PrP antibody. In some embodiments, the anti-PrP antibody is selected from one or more of 6H4, D13, D18, 8B4, 8H4, 8F9, ICSM18, 31C6, 4H11, POM1, POM2, 106, 110, P and 44B1. In some embodiments, known agent useful for treating and/or preventing prion diseases and/or brain diseases, disorders or conditions that benefit from reduced levels of the cellular prion protein (PrP^{C}) and/or altered levels of NKA alpha subunits are antisense oligonucleotides.

In some embodiments, effective amounts vary according to factors such as the disease state, age, sex and/or weight of the subject. In a further embodiment, the amount of a given agent, compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art. In some embodiments, the effective amount is one that following treatment therewith manifests as an improvement in or reduction of any disease and/or symptom thereof.

In some embodiments, the one or more agents that alter levels of one or more NKA alpha subunits, including compounds of Formula I, and/or pharmaceutically acceptable salts and/or solvates thereof are administered at least once a week. However, in another embodiment, the agents are administered to the subject from about one time per two weeks, three weeks or one month. In another embodiment, the agents are administered about one time per week to about once daily. In another embodiment, the agents are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the agents used for the treatment may increase or decrease over the course of a particular treatment regime. Changes in dosage may result and become apparent by standard diagnostic assays known in the art. In some instances, chronic administration is required. For example, the agents are administered to the subject in an amount and for duration sufficient to treat the subject.

In some embodiments, the known agents useful for treating and/or preventing prion diseases and/or brain diseases, disorders or conditions that benefit from reduced levels of the cellular prion protein (PrP^{C}) and/or altered levels of NKA alpha subunits are administered or used according to treatment protocol that is known for these agents.

In some embodiments, the subject is a mammal. In another embodiment, the subject is human. In some embodiments, the subject is a non-human animal. In some embodiments, the subject is ovine, bovine, cervine, feline or canine. Accordingly, the compounds, methods and uses of the present application are directed to both human and veterinary diseases, disorders and conditions.

In some embodiments, when two agents are used in a combination the two agents are administered contemporaneously. As used herein, “contemporaneous administration” of two agents to a subject means providing each of the two agents so that they are both active in the subject at the same time. The exact details of the administration will depend on the pharmacokinetics of the two agents in the presence of each other and can include administering the two agents within a few hours of each other, or even administering one agent within 24 hours of administration of the other, if the pharmacokinetics are suitable. Design of suitable dosing regimens is routine for one skilled in the art. In particular embodiments, two agents will be administered substantially simultaneously, i.e., within minutes of each other, or in a single composition that contains both agents. It is a further embodiment of the present application that a combination of agents is administered to a subject in a non-contemporaneous fashion. In some embodiments, two agents in a combination are used or administered simultaneously or sequentially in separate unit dosage forms or together in a single unit dosage form. Accordingly, the present application provides a single unit dosage form comprising one or more agents that alter levels of one or more NKA alpha subunits, including compounds of Formula I, and/or pharmaceutically acceptable salts and/or solvates thereof, an additional therapeutic agent, and a pharmaceutically acceptable carrier.

The dosage of the one or more agents that alter levels of one or more NKA alpha subunits, including compounds of Formula I, and/or pharmaceutically acceptable salts and/or solvates thereof, varies depending on many factors such as the pharmacodynamic properties of the agents, the mode of administration, the age, health and weight of the recipient, the nature and extent of the symptoms, the frequency of the treatment and the type of concurrent treatment, if any, and the clearance rate of the agent in the subject to be treated. One of skill in the art can determine the appropriate dosage based on the above factors. In some embodiments, an agent is administered initially in a suitable dosage that is adjusted as required, depending on the clinical response. Dosages will generally be selected to maintain a serum level of the agent from about 0.0001 μg/cc to about 10 μg/cc, or about 0.001 μg/cc to about 100 μg/cc. As a representative example, oral dosages of the one or more agents will range between about 10 μg per day to about 10 mg per day for an adult, suitably about 10 μg per day to about 5 mg per day, more suitably about 10 μg per day to about 2 mg per day. For parenteral administration, a representative amount is from about 0.00001 mg/kg to about 0.1 mg/kg, about 0.0001 mg/kg to about 0.11 mg/kg, about 0.0001 mg/kg to about 0.01 mg/kg or about 0.001 mg/kg to about 10 μg/kg will be administered. For oral administration, a representative amount is from about 0.00001 mg/kg to about 0.1 mg/kg, about 0.001 mg/kg to about 0.1 mg/kg, about 0.0001 mg/kg to about 0.01 mg/kg or about 0.001 mg/kg to about 0.01 mg/kg. For administration in suppository form, a representative amount is from about 0.001 mg/kg to about 0.1 mg/kg or about 0.001 mg/kg to about 1 mg/kg.

In embodiments of the present application, one or more agents that alter levels of one or more NKA alpha subunits, including compounds of Formula I, and/or pharmaceutically acceptable salts and/or solvates thereof, described herein may have at least one asymmetric center. Where the agents possess more than one asymmetric center, they may exist as diastereomers. It is to be understood that all such isomers and mixtures thereof in any proportion are encompassed within the scope of the present application. It is to be further understood that while the stereochemistry of the agents may be as shown in any given agent listed herein, such agents may also contain certain amounts (for example, less than 20%, suitably less than 10%, more suitably less than 5%) of agents having an alternate stereochemistry. It is intended that any optical isomers, as separated, pure or partially purified optical isomers or racemic mixtures thereof are included within the scope of the present application.

The one or more agents that alter levels of one or more NKA alpha subunits, including compounds of Formula I, and/or pharmaceutically acceptable salts and/or solvates thereof, may also exist in different tautomeric forms and it is intended that any tautomeric forms which form, as well as mixtures thereof, are included within the scope of the present application.

The one or more agents that alter levels of one or more NKA alpha subunits, including compounds of Formula I, and/or pharmaceutically acceptable salts and/or solvates thereof, may further exist in varying polymorphic forms and it is contemplated that any polymorphs, or mixtures thereof, which form are included within the scope of the present application.

The one or more agents that alter levels of one or more NKA alpha subunits, including compounds of Formula I, and/or pharmaceutically acceptable salts and/or solvates thereof, may further be radiolabeled and accordingly all radiolabeled versions are included within the scope of the present application. There, the agents also include those in which one or more radioactive atoms are incorporated within their structure.

### III. Compositions of the Application

The one or more agents that alter levels of one or more NKA alpha subunits, including compounds of Formula I, and/or pharmaceutically acceptable salts and/or solvates thereof, are suitably formulated in a conventional manner into compositions using one or more carriers. Accordingly, the present application also includes a composition comprising one or more agents that alter levels of one or more NKA alpha subunits, including compounds of Formula I, and/or pharmaceutically acceptable salts and/or solvates thereof, and a pharmaceutically acceptable carrier. In some embodiments of the application the pharmaceutical compositions are used in the treatment of any of the diseases, disorders or conditions described herein.

The one or more agents that alter levels of one or more NKA alpha subunits, including compounds of Formula I, and/or pharmaceutically acceptable salts and/or solvates thereof, are also administered to a subject, or used, in a variety of forms depending on the selected route of administration, as will be understood by those skilled in the art. In some embodiments, the one or more agents that alter levels of one or more NKA alpha subunits, including compounds of Formula I, and/or pharmaceutically acceptable salts and/or solvates thereof, are administered to the subject, or used, by oral (including sublingual and buccal) or parenteral (including, intravenous, intraperitoneal, subcutaneous, intramuscular, transepithelial, nasal, intrapulmonary, intrathecal, rectal, topical, patch, pump and transdermal) administration and the compound, salt and/or solvate, formulated accordingly. Again, conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringability exists.

In some embodiments, the one or more agents that alter levels of one or more NKA alpha subunits, including compounds of Formula I, and/or pharmaceutically acceptable salts and/or solvates thereof, are orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the one or more agents that alter levels of one or more NKA alpha subunits, including compounds of Formula I, and/or pharmaceutically acceptable salts and/or solvates thereof, are suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the one or more agents that alter levels of one or more NKA alpha subunits, including compounds of Formula I, and/or pharmaceutically acceptable salts and/or solvates thereof, and use the lyophilizates obtained, for example, for the preparation of products for injection.

In some embodiments, the one or more agents that alter levels of one or more NKA alpha subunits, including compounds of Formula I, and/or pharmaceutically acceptable salts and/or solvates thereof, are administered parenterally. Solutions of the one or more agents that alter levels of one or more NKA alpha subunits, including compounds of Formula I, and/or pharmaceutically acceptable salts and/or solvates thereof, can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, the one or more agents that alter levels of one or more NKA alpha subunits, including compounds of Formula I, and/or pharmaceutically acceptable salts and/or solvates thereof, are formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the one or more agents that alter levels of one or more NKA alpha subunits, including compounds of Formula I, and/or pharmaceutically acceptable salts and/or solvates thereof, are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the one or more agents that alter levels of one or more NKA alpha subunits, including compounds of Formula I, and/or pharmaceutically acceptable salts and/or solvates thereof, are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of the one or more agents that alter levels of one or more NKA alpha subunits, including compounds of Formula I, and/or pharmaceutically acceptable salts and/or solvates thereof, and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the one or more agents that alter levels of one or more NKA alpha subunits, including compounds of Formula I, and/or pharmaceutically acceptable salts and/or solvates thereof, are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments, the one or more agents that alter levels of one or more NKA alpha subunits, including compounds of Formula I, and/or pharmaceutically acceptable salts and/or solvates thereof, are coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, one or more agents that alter levels of one or more NKA alpha subunits, and/or pharmaceutically acceptable salts and/or solvates thereof, may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, the one or more agents that alter levels of one or more NKA alpha subunits, including compounds of Formula I, and/or pharmaceutically acceptable salts and/or solvates thereof, may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

The one or more agents that alter levels of one or more NKA alpha subunits, and/or pharmaceutically acceptable salts and/or solvates thereof, are suitably used on their own but will generally be administered in the form of a pharmaceutical composition in which the one or more agents that alter levels of one or more NKA alpha subunits, and/or pharmaceutically acceptable salts and/or solvates thereof (the active ingredient), is in association with a pharmaceutically acceptable carrier. In some embodiments, depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of one or more pharmaceutically acceptable carriers, all percentages by weight being based on the total composition.

### IV. Novel Compounds

The present application also includes all novel compounds which fall within the scope of Formula I and therefore are useful in the methods and uses described herein. In some embodiments, the present application also includes a compound of Formula I-A or a pharmaceutically acceptable salt and/or solvate thereof:

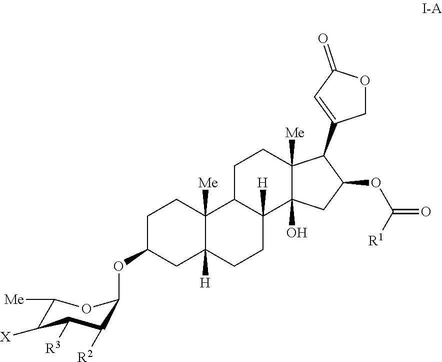

wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH and NH₂;
provided when X is ═O, R² is H and R³ is OCH₃, then R¹ is not CH₃ or CF₃.

In some embodiments, R¹ is selected from C₁₋₃alkyl and C₁₋₃fluoroalkyl. In some embodiments, R¹ is selected from CH₃, CF₃, CF₂H, CFH₂, CH₂CH₃, CF₂CF₃, CH₂CF₂H, CH₂CF₂H, CH(CH₃)₂, CF(CF₃)₂, C(CF₃)₃, and C(CH₃)₂. In some embodiments, R¹ is selected from CH₃, CF₃, CH₂CH₃, CF₂CF₃, CH(CH₃)₂, CF(CF₃)₂, C(CF₃)₃, and C(CH₃)₂. In some embodiments, R¹ is selected from CH₃, CF₃, CH₂CH₃ and CF₂CF₃. In some embodiments, R¹ is selected from CH₃ and CF₃.

In some embodiments, R² and R³ are independently selected from H, OC₁₋₃alkyl, and OC₁₋₃fluoroalkyl. In some embodiments, R² and R³ are independently selected from H, OCH₃, OCF₃, OCF₂H, OCFH₂, OCH₂CH₃, OCH₂CF₂H, OCH₂CF₂H, OCF₂CF₃, OCH(CH₃)₂, OCF(CF₃)₂, OC(CF₃)₃, and OC(CH₃)₂. In some embodiments, R² and R³ are independently selected from H, OCH₃, OCF₃, OCH₂CH₃, OCF₂CF₃, OCH(CH₃)₂, OCF(CF₃)₂, OC(CF₃)₃, and OC(CH₃)₂. In some embodiments, R² and R³ are independently selected from H, OCH₃, OCF₃, OCH₂CH₃, OCF₂CF₃, OCH(CH₃)₂ and OCF(CF₃)₂. In some embodiments, R² is selected from H, OCH₃ and OCF₃. In some embodiments, R² is H. In some embodiments, R² is selected from OCH₃ and OCF₃. In some embodiments, R³ is selected from H, OCH₃, OCF₃, OCH(CH₃)₂ and OCF(CF₃)₂. In some embodiments, R³ is selected from OCH₃, OCF₃, OCH(CH₃)₂ and OCF(CF₃)₂. In some embodiments, R³ is selected from OCH₃ and OCH(CH₃)₂. In some embodiments, R² is selected from H, OCH₃ and OCF₃ and R³ is selected from OCH₃, OCF₃, OCH(CH₃)₂ and OCF(CF₃)₂. In some embodiments, R² is H, and R³ is selected from OCH₃, OCF₃, OCH(CH₃)₂ and OCF(CF₃)₂. In some embodiments, R² is H, and R³ is selected from OCH₃, OCF₃ and OCH(CH₃)₂.

In some embodiments, X is selected from ═O and NH₂. In some embodiments, X is ═O. In some embodiments, X is NH₂.

In some embodiments, X is NH₂ and the compound of Formula I-A is a pharmaceutically acceptable salt thereof.

In some embodiments, X is selected from ═O and ═NH.

In some embodiments, when X is selected from ═O and ═NH, the compound of Formula I-A has the following structure or a pharmaceutically acceptable salt and/or solvate thereof:

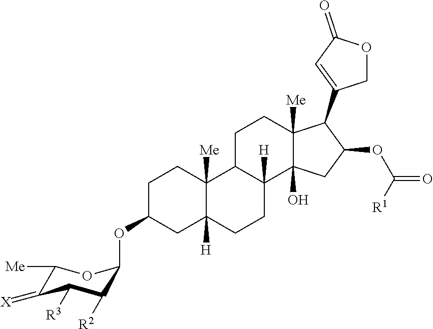

wherein
R¹ is selected from C₁₋₄alkyl and C₁₋₄fluoroalkyl;
R² and R³ are independently selected from H, OC₁₋₄alkyl and OC₁₋₄fluoroalkyl; and X is selected from O and NH;
provided when X is O, R² is H and R³ is OCH₃, then R¹ is not CH₃ or CF₃.

In some embodiments, X is ═O.

In some embodiments, the compound of Formula I-A is selected from I-1, I-2, I-5, I-6, I-7 and I-8 or a pharmaceutically acceptable salt and/or solvate thereof. Therefore, the present application also includes novel compounds I-1, I-2, I-5 or I-6 or a pharmaceutically acceptable salt and/or solvate thereof.

### V. Methods of Preparing the Compounds of the Application

Compounds of the present application or salts and/or solvates thereof, are available from commercial sources or can be prepared using methods known in the art. For example, some of the compounds of the application can be prepared using the synthetic procedures found in U.S. Pat. No. 3,898,331 (Peterson 1975), Chen et al. and Zhang, Y. et al. 2020.

Some starting materials for preparing compounds of the present application are available from commercial chemical sources. Other starting materials, are readily prepared from available precursors using straightforward transformations that are well known in the art. For example, oleandrin can be purchased from Sigma-Aldrich.

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application.

### Example 1: The Cellular Prion Protein Interacts with and Promotes the Activity of Na,K-ATPases

### A. Methods

### Antibodies

Immunoblotting made use of antibodies against PrP (MAB1562, Millipore, ON, Canada and A03213; Bertin Bioreagent, Montigny le Bretonneux, France), ATP1A1 (ab7671; Abcam, ON, Canada), ATP1B1 (GTX113390; GeneTex, CA, USA), ATP1A3 (MA3-915, Thermo Fisher Scientific, MA, USA), CD73 (HPA17357; Sigma-Aldrich, ON, Canada and ab175396; Abcam), GFAP (131-17719; Thermo Fisher Scientific), and ACTB (sc-47778; Santa Cruz Biotechnology).

### PrP Interactome Analyses

Immunoprecipitation of PrP was performed from time-controlled transcardiac perfusion crosslinked mouse brains (Schmitt-Ulms et al., 2004) using a methodology described in detail previously (Jeon & Schmitt-Ulms, 2012). The crosslinked brains were homogenized by bead beating in 150 mM Tris-HCl pH 8.3, 150 mM NaCl, 0.5% (v/v) NP-40 and 0.5% sodium deoxycholate supplemented with protease and phosphatase inhibitor cocktails (11836170001 and 4906837001; Millipore). Brain homogenates were diluted to equal total protein. The PrP-directed affinity-capture made use of the recombinant D18 antibody (sourced from Emil F. Pai laboratory, University of Toronto) conjugated to KappaSelect® beads (17-5458-01; GE Healthcare, ON, Canada). The immunoprecipitation media was extensively washed then treated with 20% acetonitrile in aqueous 0.2% trifluoroacetic acid (v/v) to recover bound protein (Ghodrati et al., 2018). After drying by centrifugal concentration, the eluates were denatured in 9 M urea then reacted with tris(2-carboxyethyl)phosphene at pH 8 and 60° C. for 30 minutes in 500 mM triethyl ammonium bicarbonate (TCEP) buffer. Upon cooling, the lysates were reacted with 4-vinylpyridine for one hour then digested with trypsin in 500 mM TEAB overnight at 37° C. Trypsin digests were reacted with iTRAQ reagents (Sciex, Concord, ON, Canada) according to the manufacturer's instructions then combined and purified on C18 microcolumns (Agilent, Santa Clara, CA, USA).

### Mass Spectrometry and Protein Sequencing

Raw data for peptide sequencing (MS and MS²) and quantification (synchronous precursor selection MS³) were acquired on an Easy-nLC 1000-Orbitrap Fusion platform. Chromatographic separation was achieved using a binary acetonitrile/water gradient operated for one (polyacrylamide gel and PVDF bands) or four (immunoprecipitates) hours. The mobile phase went from 0 to 30% acetonitrile over the first two thirds of each HPLC run, then to 100% acetonitrile for the final 10% of the run time. Proteome Discoverer 1.4 software was used for protein sequencing by Mascot and Sequest HT algorithms. False discovery rate analysis was conducted using the Percolator algorithm.

### Cell Culture

ReN VM neural progenitor (SCC008) and Neuro-2a mouse neuroblast (N2a; CCL-131) cells were obtained from Millipore and ATCC (VA, USA), respectively. The undifferentiated ReN VM cells were grown on Matrigel basement membrane matrix (354230; Corning, NY, USA) and maintained in DMEM/F12 (11320033; Thermo Fisher Scientific) supplemented with 20 ng/ml basic fibroblast growth factor (8910; Cell Signaling, MA, USA), 20ng/ml epidermal growth factor (RKP01133; Reprokine, FL, USA), 10 Units/ml heparin sodium salt (H3149; Sigma-Aldrich) and 1× N21-MAX (AR008; R&D Systems, MN, USA) or 1× B27 (17504044; Thermo Fisher Scientific) media supplement. The cells were differentiated into neuronal and glial populations with the removal of heparin and growth factors from media for at least 7 days. Passage-matched N2a and RML infected N2a cells were created as described previously ( ) and maintained in DMEM (11995065; Thermo Fisher Scientific) supplemented with 10% fetal bovine serum (12483020; Thermo Fisher Scientific) and 1% GlutaMAX® (35050061; Thermo Fisher Scientific). CRISPR-generated N2a knockout cells were described previously (Mehrabian et al., 2014). Mouse brains harvested from early (70DP1) and late (132DP1) RML infection timepoints were a kind gift from Dr. Kurt Giles (University of California San Francisco).

### Immunocytochemistry

ReN VM cells were cultured on glass coverslips according to the procedure described above. Following 7 days of differentiation, cells were fixed with 4% paraformaldehyde (w/v in PBS), followed by incubation with permeabilization and blocking buffer (0.1% Triton X-100 (v/v), 1% BSA (w/v) in PBS). Cells were incubated overnight at 4° C. with the PrP-directed and ATP1A1-directed antibodies added at a 1:100 dilution. Upon removal of the primary antibodies, cells were incubated at 1:400 dilutions for 90 minutes at ambient temperature with secondary antibodies conjugated to fluorescent dies that emit at 647 and 488 nm wavelength for the detection of ATP1A1 and PrP, respectively. Next, cover slips were mounted onto glass slides using ProLong Gold with DAPI antifade mounting agent. Deconvolution was undertaken and the degree of co-localization was computed using built-in algorithms within the ‘Zen black’ microscopy analysis software (version 8.1.0.484, Zeiss Microscopy, Jena, Germany).

### CRISPR/Cas9 Knockout of PrP in ReN Cells

The methodology to develop knockout clones was as described previously (Mehrabian et al., 2014). Briefly, the selected gRNA human PrP (CACTGGGGGCAGCCGATACCCGG) was cloned into the gRNA plasmid (MLM3636; Addgene, MA, USA) following digestion with the BsmBI enzyme (R0580S; New England BioLabs, MA, USA) and verified by Sanger sequencing. The gRNA was then transfected into the previously transduced ReN VM cells with the Cas9 enzyme using Lipofectamine Stem Transfection reagent (GST-2174; Thermo Fisher Scientific). After 48 hours, cells were diluted and grown to form single cell-based colonies and screened for PrP expression by immunoblotting.

### ⁸⁶Rb⁺ Uptake Assay

Cells were grown in 24-well format plates to 90% confluency. Media were changed to plain DMEM-F12 three hours before the ⁸⁶Rb⁺ uptake measurements. Each condition was analyzed in the presence and absence of 1 mM ouabain and in three biological replicates. Following the addition of 2 μCi 86Rb (NEZ072; Perkin Elmer, MA, USA) per well, cells were incubated at 37° C. for 10 minutes and then the uptake stopped by 4 cold washes of 100 mM MgCl₂ and 10 mM HEPES-Tris buffer (pH 7.4). Cells were then lysed in 1% NP40, 150 mM NaCl and 150 mM Tris (pH 8.3). Following the total protein content measurement using BCA assay, the readout is reported in nmol Rb uptake/min/mg.

### Western Blot Analyses

In order to harvest cultured cells, they were washed with cold PBS once and then, lysed in cold lysis buffer containing 150 mM NaCl, 150 mM Tris-HCl (pH 8.3), 1% NP40 plus protease and phosphatase inhibitors. Brain homogenates were created in lysis buffer containing 150 mM NaCl, 150 mM Tris-HCl (pH 8.3), 0.5% NP40 and 0.5% sodium deoxycholate supplemented by protease and phosphatase inhibitors. In both cases, samples were spun down to remove the insoluble debris and protein concentration was determined using bicinchoninic acid (BCA) colorimetric protein assay kit (23225; Thermo Fisher Scientific). Equal amounts of total protein were loaded and run on Bis-Tris denaturing gels (NW04125BOX; Thermo Fisher Scientific) and transferred to Polyvinylidene fluoride membranes (PVDF, IPVH00010, Millipore). The membranes were blocked in 10% skim-milk (in TBST-tween) and probed with the primary antibodies overnight at 4° C. The next day the blots were washed in TBS-tween, three times and incubated with the relevant secondary antibodies (7074S, 7076S; Cell Signaling Technologies; MA, USA) for an hour at room temperature followed by three times of TBS-tween wash to remove the unbound antibodies. The signals were development using either X-ray films or a LI-COR Odyssey Fc digital imaging system (LI-COR Biosciences, NE, USA).

### Inhibitor Treatments of ReN VM Cells

Ouabain octahydrate (O3125) and Calpain Inhibitor I (A6185) were purchased from Sigma-Aldrich and dissolved in water and DMSO respectively to generate stock solutions with 1000× working concentrations prior to the treatment. The treatments were renewed daily for the indicated duration.

When specified, differentiated ReN VM cells were treated with cardiotonic steroid compounds ouabain for 3 or 7 consecutive days with daily renewal of half of differentiation media plus the inhibitors. When applicable, calpain inhibitor I (A6185) was added 2 h before the initial treatment of ouabain at a final concentration of 20 μM and replenished daily along with half media change on differentiated ReN VM cells.

### RT-qPCR Analyses

To quantify ATP1A1 and PRNP transcript levels in ReN VM cells following vehicle or ouabain treatment at 10 nM, 30 nM and 50 nM conditions, cells were treated every other day over a period of 7 days in biological triplicates and qPCR was conducted in technical duplicates, each reaction containing 50 ng of cDNA, TaqMan master mix (catalog number 4427788, Thermo Fisher) and one of the following TaqMan assay kits (Thermo Fisher) targeting of human PRNP (catalog number Hs01920617_s1), ATP1A1 (catalog number Hs00933601_m1), ATP1A2 (catalog number Hs01560076_g1) or ATP1A3 (catalog number Hs00958036_m1). Transcript abundance was normalized using the reference genes PPIA (catalog numberHs99999904_m1) and GAPDH (catalog number (Hs03929097_g1). Negative controls included template controls for each gene expression assay along with a no probe control that all produced no signal of transcript amplification

### Identification of Ouabain Dependent 5′-Ectonucleotidase Upregulation

Lysates from ouabain treated and mock treated ReNcell VM cultures were subjected to SDS-PAGE and western blot. From lanes containing the treated and mock treated samples, Coomassie blue stained bands of approximately 60 KDa were excised and destained with 50% acetonitrile in 100 mM NH₄ HCO₃ (polyacrylamide) or 25% acetic acid in 50% methanol (PVDF). Polyacrylamide bands were stored at 60° C. in 10 mM dithiothreitol for 15 minutes, then in the dark for 20 minutes in 15 mM iodoacetamide. PVDF bands were treated with 0.5% polyvinylpyrrolidone (w/v) in 100 mM acetic acid at 37° C. with shaking at 300 rpm for 1 hour then washed with water. The bands were each covered in 125 ng of trypsin (Promega, Madison, WI, USA) in 100 mM NH₄HCO₃ at 37° C. overnight and the resulting peptides extracted with 50% acetonitrile (v/v) in 100 mM NH₄HCO₃. The extracts were dried in a centrifugal concentrator, resuspended in 0.1% formic acid (v/v) then concentrated on C18 microcolumns (Agilent, Santa Clara, CA, USA) and analyzed by LC-MS/MS.

Proteins sequenced from polyacrylamide or PVDF bands were sorted first according to the number of spectral counts by which they were identified from ouabain treated samples and second by the ratio of peptide to spectral matches in ouabain treated samples relative to mock treated samples. NTSE (Uniprot Accession P21589-2) was the most enriched protein in ouabain treated samples based on this ranking.

### B. Results

### The Molecular Environment of PrP^{C} in Mouse Brains is Dominated by NKAs

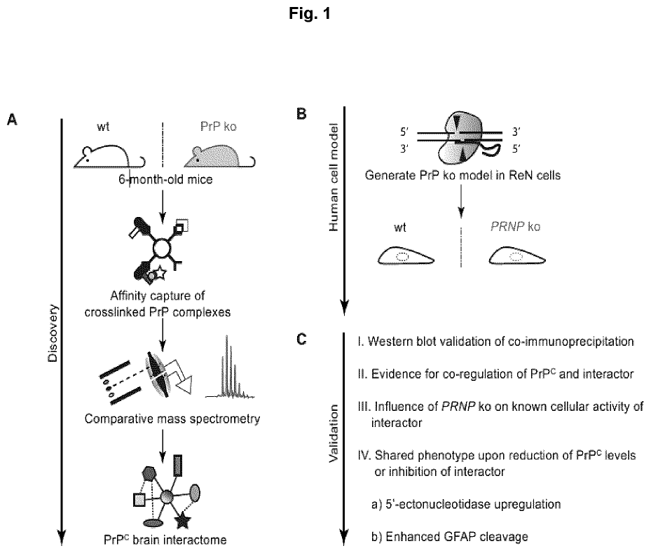

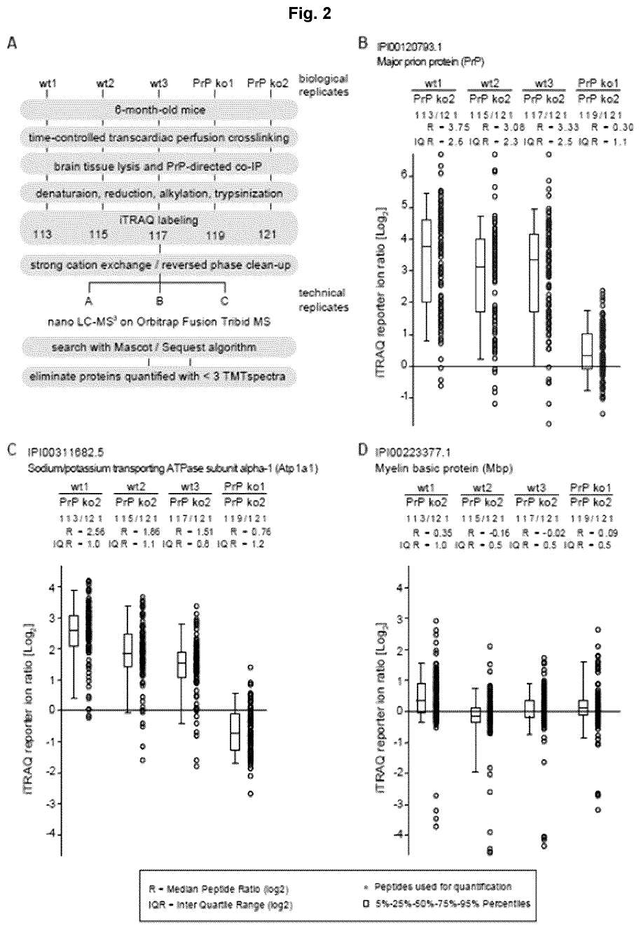

FIG. 1 shows the study design of Example 1. To facilitate the capture of proteins that reside in vivo in the mouse brain in spatial proximity to PrP^{C}, protein interactions were stabilized prior to brain homogenization through time-controlled transcardiac perfusion crosslinking (Jeon & Schmitt-Ulms, 2012){(Schmitt-Ulms, 2004). The affinity capture of PrP^{C} and its crosslinked molecular neighbors made use of a recombinant humanized antibody (D18) known to target a non-linear epitope comprising PrP residues 133-157. The sample handling of co-immunoprecipitates included stringent washing of affinity capture matrices to minimize non-specific binding (Markham, Bai & Schmitt-Ulms, 2007). To maximize interactome coverage, equal aliquots of trypsin digested eluate samples were initially subjected to separate liquid chromatography/tandem mass spectrometry analyses and relative quantitations were based on the spectral counting method (Lundgren et al., 2010). Three biological replicates constituted separate PrP^{C} co-immunoprecipitations from distinct wild-type brains, with identically processed samples from Prnp^{-/-} brains serving as negative controls (FIG. 2A). Mass spectra were stringently filtered using the ‘Percolator’ discriminator routine within ProteomeDiscoverer (version 1.4.0.288) and matched to the mouse international protein index (IPI; version 3.87) database using embedded Mascot and Sequest HT algorithms. The spectral counting-based analysis of unlabeled tryptic digests of co-immunoprecipitates revealed 384 proteins whose levels were more enriched in PrP-specific samples than in Prnp^{-/-} derived controls. Indicative of low non-specific protein contaminant levels, the prion protein was observed with 168 peptide-to-spectrum matches (PSMs) in wild-type and only 16 PSMs in Prnp^{-/-} derived control eluates, giving rise to the highest difference count of 152 PSMs (wild-type minus control) observed for any protein in the dataset. When all proteins were sorted by their difference spectral counts, the neural cell adhesion molecule 1 (Ncam1) was the most co-enriched interactor, and its paralog Ncam2, as well as the closely related proteins contactin-1, the neural cell adhesion molecule L1 and neurofascin, were amongst the top ten-listed proteins. In the top ten group of PrP candidate interactors were also two alpha subunits of Na,K-ATPases (NKAs), namely alpha-1 and alpha-3, the latter known to be predominantly expressed in neurons. Additional NKA subunits, comprising Atp1a2, Atp1b1 and Atp1b2 were in the list of the 40 most co-enriched proteins. However, whereas Ncam1 and other members of the immunoglobulin superfamily, which also included cell adhesion molecule 3 (Cad3), myelin-associated glycoprotein (Mag), neuronal cell adhesion molecule (Nrcam) and immunoglobulin superfamily member 8 (Igsf8), exhibited low non-specific binding to the affinity matrix, NKA subunits were also relatively abundant in the negative control, consistent with the interpretation that the presence of these pumps in the PrP-specific eluates reflects both PrP-dependent and PrP-independent binding to the affinity matrix. The list of the 40 most co-enriched proteins also contained: 1) subunits of fructose-bisphosphate aldolase (Aldoa, Aldoc); 2) synaptic fusion complex subunits (Snap25, Syt1, Stx1b, Sv2b); 3) dipeptidyl peptidases (Dpp6 and Dpp10); 4) a ubiquitin-like modifier-activating enzyme 1 (Uba1); 5) the amyloid precursor protein (APP); 6) subunits of other ATPases (Atp6v0a1, Atp2b1, Atp2b2); 7) heat shock protein 90 (Hsp90ab1 and Hsp90aa1); 8) the excitatory amino acid transporter 2 (Slc1a2); 9) peptidyl-prolyl cis-trans isomerase A; and 10) the metabotropic glutamate receptor.

Next, isobaric labeling with iTRAQ reagents followed by multiplexing was undertaken with a view to minimize run-to-run variance and consolidate relative quantitations of the more abundant proteins (Zieske, 2006). Identical procedures had previously been successfully employed to study PrP^{C} interactors in four murine cell models (Ghodrati et al., 2018). The relative enrichment of proteins in iTRAQ-labeled wild-type samples versus PrP knockout controls was computed using the ‘Reporter Ion Quantifier’ algorithm within Proteome Discoverer. The ‘Major prion protein’ was again the highest ranked hit with >800 PSMs, a correlate for relative abundance (Lundgren et al., 2010). More than hundred iTRAQ-reporter ion ratios, collected after the high-energy fragmentation of the most intense ions observed in a given PSM, validated the expected PrP enrichment in wild-type versus Prnp^{-/-} derived samples (FIG. 2B). Note that despite the absence of PrP in the Prnp^{-/-} derived samples, the average PrP enrichment ratio is not infinite (as would be mathematically expected) but approximates a value of 10 (Log₂ 3.3) due to the existence of background noise in all iTRAQ reporter ion channels, purity limitations of iTRAQ chemistry and the inadvertent co-isolation of contaminating ions observed in complex peptide mixtures that work together to artificially limit the dynamic range. Ncam1 was also again highly enriched, its peptides exhibiting a median >8-fold (Log₂ 3.0) enrichment over negative controls.The two proteins whose PSM count was next highest in the multiplex analyses were the alpha-3 subunit of a sodium/potassium-transporter ATPase (Atp1a3) and myelin basic protein (Mbp). However, whereas iTRAQ reporter ion ratios revealed Atp1a3 to co-enrich with PrP (FIG. 2 C), the myelin basic protein exhibited no selective co-enrichment, identifying it as a non-specific binder to the affinity matrix (FIG. 2 D).

Taken together, these data corroborated Ncam1 as a predominant PrP interactor but also put a spotlight on NKAs, indicating that these pumps may be abundant in spatial proximity to PrP^{C} in the brain.

### NKAs and PrP Interact and Their Levels are Correlated

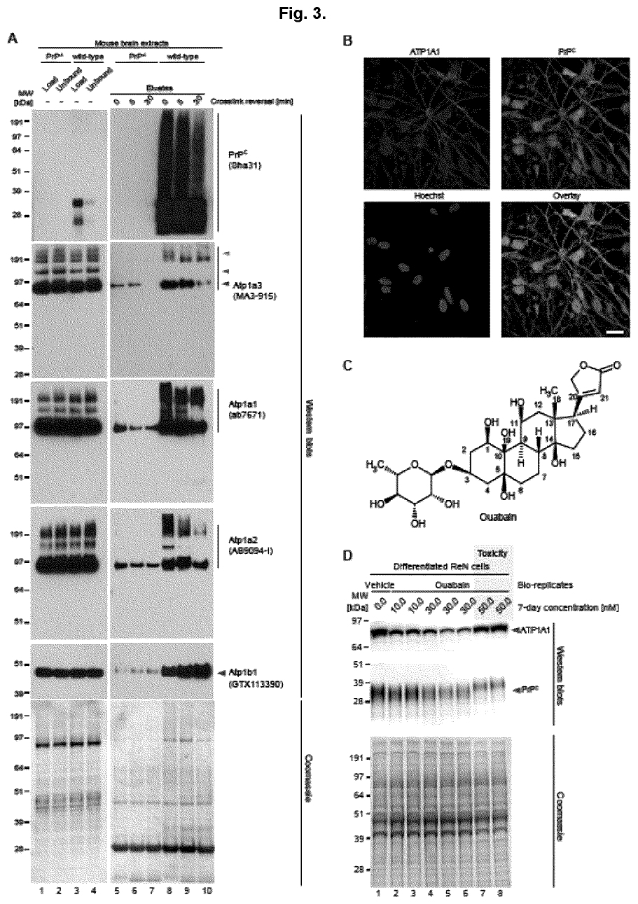

To validate the putative interaction between PrP^{C} and NKAs, brain homogenates generated from in vivo crosslinked mouse brains were subjected to small-scale PrP-directed co-immunoprecipitation followed by western blot analyses. As negative controls served side-by-side produced samples from Prnp^{-/-} mice (FIG. 3A). This experiment corroborated that small amounts of alpha (Atp1a3) and beta (Atp1b1) subunits of NKAs bind non-specifically to the affinity capture matrix, as the mass spectrometry analyses had revealed. Further, the western blot data also validated the PrP-dependent binding of NKAs. Moreover, a comparison of the banding pattern of extract and eluate fractions obtained with the Atp1a3-directed western blot provided evidence that the presence of NKAs in PrP-specific eluate fractions cannot be ascribed to trivial carryover of NKAs from extracts into eluates. If the latter had been the case, eluate signals would be expected to give rise to the same Atp1a3 western blot band pattern as the input samples. This was not the case, as evidenced by the absence of the band highlighted by an unlabelled arrowhead in eluate fractions and differences in the relative abundances of bands detected with the Atp1a3-directed antibody. Heating the eluate fractions at 90° C. in the presence of β-mercaptoethanol for 5 or 30 minutes caused a subset of high molecular weight bands to disappear (e.g., the 250 kDa band detected with the Atp1a3 antibody in lane 8 that is absent in lanes 9 and 10), consistent with the interpretation that they comprised crosslink products.

To assess the co-localization of Atp1A1 and PrP^{C}, a human neuronal cell model (Ren VM cells) known to harbor a stable human male karyotype (46, XY) and to differentiate into co-cultures of neurons, astrocytes and oligodendrocytes upon growth factor removal was employed (Donato et al., 2007). To this end, the cells were immunolabeled with antibodies directed against endogenous Atp1a1 PrP^{C} and their localization captured by fluorescently tagged secondary antibodies. These analyses established robust co-localization of both proteins in signal overlays, validating that both proteins ddi not acquire the ability to interact only upon cell homogenization.

The expression of proteins that engage in functional associations is often coordinated. To begin to assess the extent to which the biology of PrP^{C} and NKAs may be interlinked, the effect of CGs on the steady-state levels of NKAs, their natural targets, and on PrP^{C} was explored. The experiment made use of ouabain, a plant derived cardiotonic steroid (FIG. 3 B), because it is understood to have low membrane penetrance and therefore may predominantly affect NKAs at the cell surface. Perhaps not surprisingly, one-week exposure of differentiated ReN VM cells to a range of nanomolar concentrations of ouabain reproducibly altered levels of NKAs, here shown for the ATP1A1 subunit. More specifically, a bimodal effect was observed, whereby exposure of cells to low nanomolar levels of ouabain (approximately up to its IC50 of 15-41 nM (Lucchesi & Sweadner, 1991)) resulted in a ouabain concentration-dependent depletion of ATP1A1, yet in the presence of toxic ouabain concentrations, ATP1A1 levels bounced back, which, while not being bound by theory, possibly reflects a feedback rescue attempt by a cell whose electrochemical gradient is under threat. Interestingly, levels of PrP^{C} correlated closely with ATP1A1 levels in the presence of non-toxic ouabain concentrations. No such correlation was observed for a majority of other proteins in the cell extract, as evidenced by Coomassie analyses. Intriguingly, at toxic ouabain concentrations, PrP^{C} was observed to migrate slower during denaturing SDS-PAGE separation, reminiscent of it being more fully N-glycosylated (FIG. 3 C).

### PrP Expression Directly Modulates NKA Activity

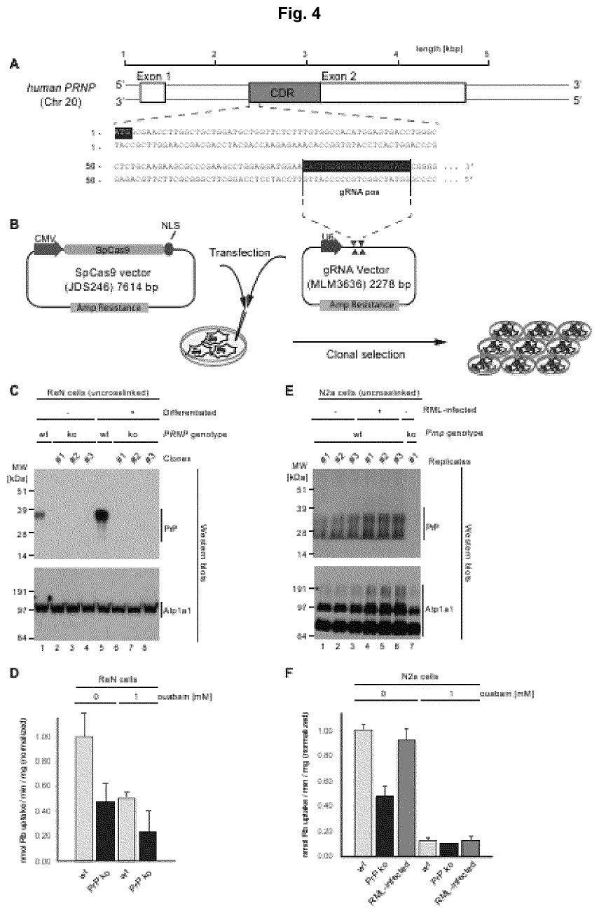

To further investigate if the co-enrichment of NKAs in the PrP interactome dataset reflects a functional relationship between PrP^{C} and NKAs, PrP-deficient derivative lines of the ReN VM cells were generated. More specifically, a CRISPR-Cas9 gene knockout strategy was employed to generate several other PrP-deficient mouse cell lines (Mehrabian et al., 2014) and a genome stretch at the 5′ end of the PrP open reading frame was targeted (FIG. 4A), capitalizing on the host-encoded non-homologous end-joining repair program to induce a frame shift and premature stop codon in the PRNP gene upon co-transfection of gRNAs and Cas9-expressing plasmids (FIG. 4 B). Immunoblot analyses validated the successful generation of several PrP knockout clones and established that the mere absence of PrP in this cell line did not alter the expression of the alpha-1 subunit of the NKA (ATP1A1) (FIG. 4 C). Next, differentiated wild-type and PrP-deficient ReN VM cells in their NKA activity were compared using a well-established radioisotope assay that capitalizes on the promiscuous uptake of potassium or rubidium (⁸⁶Rb⁺) by the pump. To determine the contribution of NKAs to the total cellular ⁸⁶Rb⁺ uptake activity, control samples were exposed to pump-saturating 1 mM concentrations of ouabain (FIG. 4 D). Wild-type ReN VM cells took up approximately 0.34 nmol of ⁸⁶Rb⁺ per min and mg of total cellular protein. The complete block of NKAs reduced this uptake by 49%, indicating that uptake through NKAs accounts for approximately half of the total ⁸⁶Rb⁺ internalization in this cell model.

Relative to wild-type cells, PrP-deficient cells exhibited a 52% reduction in the ⁸⁶Rb⁺ uptake rate. Ouabain exposure of PrP ko cells further halved this uptake, bringing it to 24%, indicating that the presence of PrP promotes ⁸⁶Rb⁺ uptake. Moreover, these results suggested a majority of the ⁸⁶Rb⁺ uptake that the presence of PrP promoted was mediated by NKAs. The latter point was even more convincingly conveyed when the analyses were repeated with mouse neuroblastoma cells (N2a). In the latter model, the ouabain-resistant ⁸⁶Rb⁺ uptake as observed to account for only 13% of total uptake, yet PrP-deficiency again reduced uptake rates by 53%, strongly arguing that PrP^{C} predominantly promoted the ouabain-sensitive uptake through NKAs. An additional assessment of whether infection with mouse-adapted scrapie (RML) prions influences NKAs activity revealed an 8% reduction in total ouabain-sensitive ⁸⁶Rb⁺ uptake rate in RML-infected N2a cells. Results in this section pointed to a role of PrP^{C} promoting NKAs activity. Consequently, either its removal (in PrP-deficient cells) or its perturbation (in prion disease) reduces the activity of NKAs, thereby mimicking the presence of CGs.

### Increased 5′-Nucleotidase Levels in the Presence of CGs or in Cells Devoid of PrP

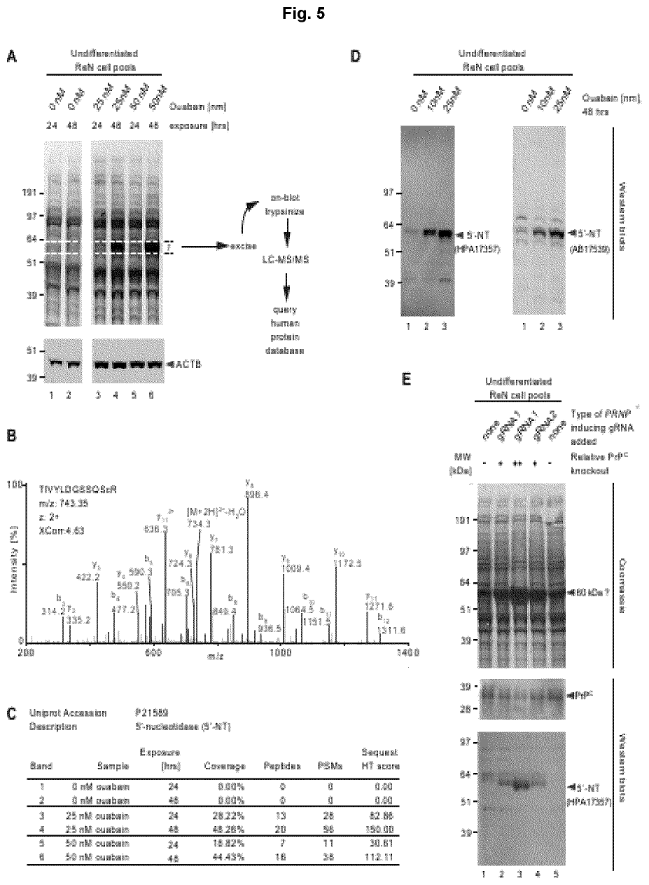

When experimenting with CGs, it was noticed that prolonged exposure (48 hrs) of undifferentiated ReN VM cells to non-toxic low nanomolar levels of ouabain caused a pronounced upregulation of a band that migrated in the denaturing SDS-PAGE with an apparent molecular mass of 60 kDa (FIG. 5A). Alert to the possibility that the identity of this signal could serve as an endogenous reporter of NKA inhibition, its identification was pursued. To this end, the band together with identically-sized control PVDF pieces were excised from the same level of the blot but originating from the analysis of cells which had not been exposed to ouabain, undertook on-blot trypsinization, and LC/MS/MS analyses. Queries of the human UniProt® database with tandem mass spectra collected in each of a total six sample and control analyses revealed the signal to be derived from 5′-ectonucleotidase (5′-NT). This conclusion could be drawn on the basis of dozens of highly confident PSMs obtained for this protein (FIG. 5B) and the fact that its presence or absence and relative abundance (approximated on the basis of its PSM counts) correlated well with the intensity of the corresponding band-of-interest in the Coomassie-stained blot membrane (FIG. 5C). This assignment was validated by immunoblotting of non-treated and ouabain treated samples with two 5′-NT-specific antibodies, which recognize non-overlapping epitopes within this protein. As expected, this analysis unequivocally confirmed steady-state levels of 5′-NT to increase following 48 hrs exposure of undifferentiated ReN VM cells to low nanomolar ouabain levels (FIG. 5D). In parallel to these analyses, it was noted that a band of the same apparent molecular mass was also increased in Coomassie analyses of pools of ReN VM cells, in which the PrP gene had been targeted by CRISPR-Cas9 knockout gRNAs. Moreover, the increase in signal intensity for this band correlated inversely with the steady-state levels of PrP in these samples. 5′-NT-directed immunoblot analyses of the respective fractions revealed this band to indeed also comprise 5′-NT (FIG. 5E). These results corroborated the conclusion that PrP deficiency partially impairs NKA activity and, as such, acts as a molecular mimic of cellular exposure to CGs.

### Exposure of ReN VM Cells to CGs Increases Levels of GFAP and its Calpain-Dependent Fragments

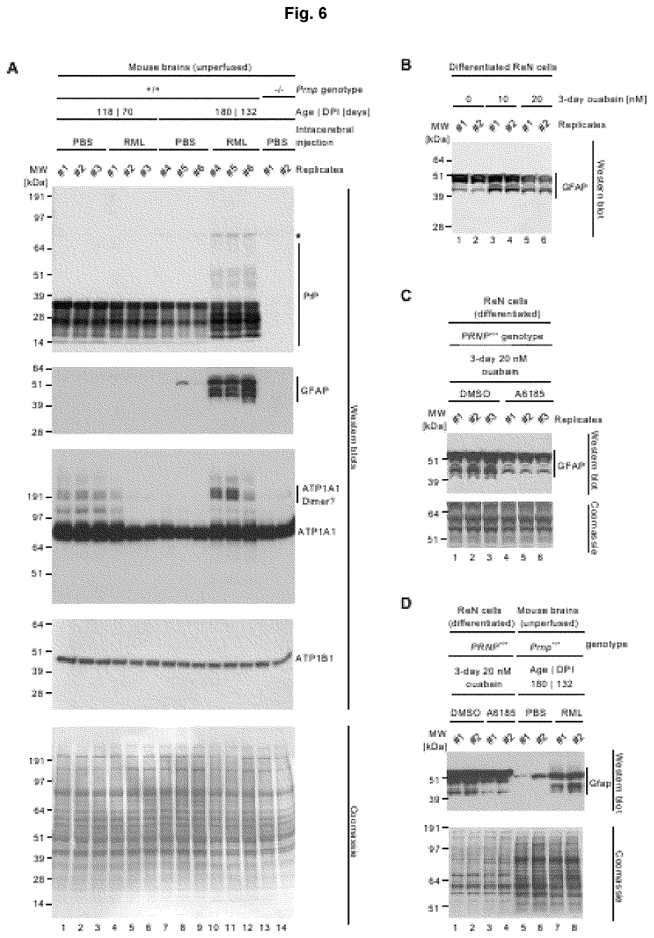

To begin to assess if partial inhibition of NKAs may play a role in prion diseases, differentiated ReN VM cells which represent co-cultures of neurons, astrocytes and oligodendrocytes were used, thereby providing a paradigm suitable for observing shifts in the differentiation of these three main lineages of brain cells. Aside from the conversion of the prion protein that is central to the etiology, one hallmark shared amongst prion diseases is a profound astrogliosis. At the molecular level, the latter is characterized by an increase in the expression of the glial fibrillary acidic protein (GFAP) and the appearance of smaller bands that are reactive to GFAP-directed antibodies (Gray et al., 2006). To begin to assess a possible role of NKAs in this molecular phenotype, mice were infected with RML prions and the effect of prion disease on levels of GFAP and NKAs was investigated. This analysis revealed the expected increase in GFAP levels in six months old mice that had been RML infected (sacrificed at 132 DPI). Intriguingly, the increase in GFAP levels was paralleled by an increase in the levels of Atp1a1 antibody-reactive bands, most strikingly observed at the level of high molecular weight bands that possibly represented dimers or SDS-stable complexes (FIG. 6A). Next, if exposure of differentiated ReN VM cells to low levels of ouabain can mimic the GFAP cleavage in vitro was investigated. Indeed, three days following addition of nanomolar levels of ouabain to the cell culture medium, an increase in the intensity levels of bands that were reactive toward GFAP-directed antibodies, including the pronounced appearance of faster migrating signals were observed (FIG. 6B). It is well established that inhibition of NKAs leads to an increase in intracellular calcium levels through stimulation of the reverse mode activity of the Na,Ca-exchanger. This in turn is known to activate calcium-dependent signaling. Because a previous report had shown that GFAP can be a target of calcium-dependent calpain proteases (Lee et al., 2000), it was hypothesized, that the lower mass GFAP-antibody-reactive bands might represent endoproteolytic calpain cleavage products, a model consistent with a previously reported activation of calpain levels in the hippocampus of prion-infected mice (Gray et al., 2006). To explore this concept experimentally, a membrane permeable calpain inhibitor (N-acetyl-Leu-Leu-norleucinal, also known as A6185) was added to a subset of cell culture dishes that were exposed to ouabain (FIG. 6C). A GFAP-directed antibody revealed that the concomitant addition of the calpain inhibitor had blocked the excessive formation of faster migrating bands, consistent with an endoproteolytic mechanism of their formation. The side-by-side analysis of total cellular protein extracts from prion-infected brains and ouabain-treated ReN VM cells revealed considerable similarities in GFAP antibody reactive band patterns. Taken together, these data implicated the Cat -dependent activation of calpain endoproteases in the ouabain-induced GFAP proteolysis and suggest that a similar course of events may underlie the GFAP cleavage observed in prion diseases (FIG. 6D).

### CG-Induced Changes to PrP^{C} Levels Correlate with ATP1A1 and 2 but not ATP1A3 Isoform Levels

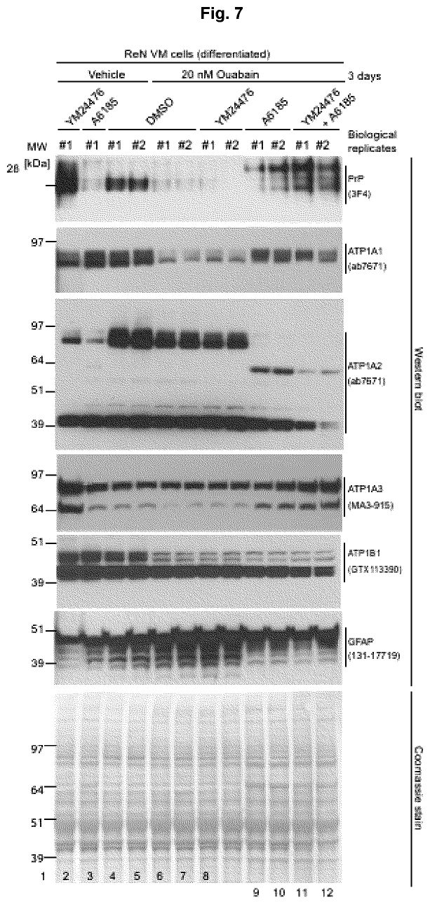

One of the best-characterized consequences of CG inhibition of NKAs is an increase in intracellular calcium levels. The latter is mediated by the ensuing increase in intracellular Na⁺ concentrations driving an accumulation of intracellular Ca²⁺ levels through the activity of nearby Na,Ca-exchangers operating in reverse mode (Dostanic et al., 2004). In fact, this phenomenon forms the basis for the CG-centered treatment of heart diseases that rely on the fortification of the calcium-dependent contraction of heart muscles. Whereas expression of ATP1A4 is largely restricted to the testis (Woo et al., 2002), the expression of ATP1A1 is widespread, and ATP1A2 and ATP1A3 are observed in several tissues, including the brain. There, ATP1A3 is predominantly found in neurons, and ATP1A2 is abundant in astrocytes (Murata et al., 2020). Whether CG exposure of ReN VM cells affects the steady-state levels of the three brain-expressed NKA α subunits equally was next investigated. Moreover, to determine if any CG-effect on their expression levels depends on the action of the Na,Ca-exchanger, the CG-treatment of ReN VM cells in the presence or absence of a well-known inhibitor (YM24476) of the reverse mode activity of the Na,Ca-exchanger was repeated. Results from this experiment provided evidence that the CG-dependent reduction in steady-state PrP^{C} levels does not depend on an increase in intracellular calcium levels, because blocking the reverse mode activity of the Na,Ca-exchanger did not affect this outcome (FIG. 7). This experiment also established that the CG-dependent reductions in steady-state PrP^{C} levels parallel the corresponding level changes seen for ATP1A1. ATP1A2 levels also were reduced in the presence of CGs but to a lesser degree, whereas the most prominent western blot signal for ATP1A3 was not affected by prolonged cellular exposure to 20 nM ouabain.

### C. Discussion

As recent work identified cell type-specific differences in the proteins PrP^{C} binds to (Ghodrati et al., 2018), the molecular environment of PrP^{C} in the brain using refined tools and methodology was further investigated. Surprisingly, subunits of NKAs followed Ncam1 in this analysis as the top-ranked PrP^{C} interactors on the basis of both their profound co-enrichment and hundreds of peptide-to-spectrum matches assigned to them. In subsequent work, it was validated that PrP^{C} and NKAs do indeed coimmunoprecipitate when captured from in vivo crosslinked brain extracts. Using the human ReN VM cell model evidence of partial colocalization of PrP^{C} with ATP1A1 was produced and established that the steady-state levels of these proteins are correlated in the presence of nanomolar concentrations of cardiac glycosides (CGs). Next, it was found that PrP^{C} promotes NKA activity using a direct ion uptake assay in ReN VM cells and mouse neuroblastoma cells. Corroborating these results, the knockout of PrP^{C} expression was observed to mimic a molecular phenotype of 5′-NT upregulation that can also be induced by partial inhibition of NKAs with CGs. Moreover, the cellular exposure to CGs can cause a calpain-dependent cleavage of the astrocytic intermediate filament protein GFAP that resembles a previously observed and here reproduced cleavage signature for this protein in prion-infected mice. Consistent with this interpretation, when inhibiting the calpain activation that occurs in the presence of CGs GFAP cleavage was prevented.

PrP^{C} has been reported to control NCAM1 polysialylation (Mehrabian et al., 2015) and to be embedded in a specialized membrane domain enriched in molecules known to modulate TGF and integrin signaling in murine cell models (Ghodrati et al., 2018). Here it was showed that murine brain PrP^{C}, although still resident in proximity to NCAM1 and related cell adhesion molecules rich in Ig-like domains (Cntn1, Ncam2, L1 cam, Nfasc, Cadm3, etc), is primarily surrounded by proteins that utilize ATP to polarize brain cells (NKAs, Ca²⁺ ATPase, V-type H⁺ ATPase), or are part of the synaptic vesicle fusion apparatus (SYT1, SNAP25, SV2B).

At this time missing is information regarding the NKA-PrP^{C} binding interface. NKA alpha-subunits are well-characterized to contribute a multi-span transmembrane domain and large cytosolic domains to the heteromeric NKA complexes (Shinoda et al., 2009; Laursen et al., 2013). On the basis of topology and structural considerations, one may therefore predict that the indispensable beta-1 subunit contributes to the PrP^{C} binding interface. Not only is this protein, like PrP^{C}, facing the extracellular space but its fold is also structurally related to the immunoglobulin-like and fibronectin-like IIII domains observed in NCAM1 and other members of the immunoglobulin superfamily of proteins (Shinoda et al., 2009; Bab-Dinitz et al., 2009), whose PrP^{C} binding has been mapped (Schmitt-Ulms et al., 2001) and partially resolved by NMR analyses (Slapsak et al., 2016).

In order to elucidate the significance of this interaction, a human co-culture model of neurons, astrocytes and oligodendrocytes was used show that PrP^{C} promotes the activity of NKAs it interacts with. Consistent with this interpretation, removal of PrP^{C} caused a reduction in ion uptake activity and mimicked the addition of cardiotonic steroids in regard to an upregulation of 5′-NT. Interestingly, an increase in the activity of ATP hydrolyzing ectonucleotidases has previously been described in hippocampal (but not in cortical) synaptosome preparations of Prnp null mice (Pereira et al., 2001). How does exposure of CGs activate 5′-NT? Although the answer to this question has not been firmly established, while not wishing to be limited by theory, one plausible scenario is as follows: NKA inhibition is well known to promote the accumulation of intracellular Ca²⁺ through the passively operating Na⁺/Ca²⁺ exchanger that relies on the pump-generated Na⁺ gradient for exporting Ca²⁺ from the cytosol but operates in reverse mode when the normal electrochemical gradient has been compromised. Even low concentrations of ouabain (or other CGs), insufficient for blocking ion transport by the pump, have been reported to cause local Ca²⁺ increases, possibly through a signaling complex that involves the inositol 1,4,5-trisphosphate receptor (IP3R) (Aperia et al., 2016). Several authors have reported that changes in calcium levels transmit to neighboring cells, a phenomenon relying on gap junctional intercellular communication (Lapato & Tiwari-Woodruff, 2018). Others have reported that the detachment of gap junction hemichannels composed of connexins, which can be invoked by elevated calcium, leads to cellular ATP leakage into the extracellular space (Kang et al., 2008; Donahue, Qu & Genetos, 2017; Burnstock & Knight, 2017). The spike in extracellular ATP would be expected to trigger the expression of 5-NT (Brisevac et al., 2015), the final enzyme in an extracellular cascade of ATP dephosphorylation steps (Yegutkin, 2008). Consistent with this model, 5′-NT plays a role in the response to vascular leakage (Thompson et al., 2004) and serves as a marker of mesenchymal stem cells (Barry et al., 2001; Shahbazi et al., 2016), its expression being dramatically upregulated during epithelial-to-mesenchymal transition (Lupia et al., 2018; Yu et al., 2017; Mehrabian et al., 2015; Xiong et al., 2014), when cell-to-cell contacts are broken and cells move to integrin-mediated cell substrate adherence (Adzic & Nedeljkovic, 2018).

Interestingly, the expression of 5′-NT was also observed to directly correlate with astrogliosis (Barros-Barbosa et al., 2016), which cumulatively suggest that PrP^{Sc} may inhibit NKA uptake activity (FIG. 3 F and FIG. 5 C)—despite an increase in total ATP1A1 signal intensity that was observed in prion-infected ScN2a cells (FIG. 3 E). In light of the above, the gradual poisoning of NKAs by PrP^{Sc} could mimic CG exposure and explain the increase in GFAP that has repeatedly been observed in prion diseases, together with the enhanced calpain cleavage (Llorens et al., 2017; Yadavalli et al., 2004). Notably, this calpain activation would not only produce GFAP cleavage products but also downregulate PrP^{C}, thereby removing the essential substrate for prion conversion and slowing the disease, a phenomenon proposed to underlie the extraordinary long incubation periods observed in prion diseases (Mays et al., 2015). Moreover, proteins that shareseveral biochemical characteristics and raft-residence with PrP^{C}, the enhanced local calpain activity might account for its concomitant prion disease-associated depletion (Watts et al., 2007). Finally, this chain of events, whereby PrP^{Sc} poisons and draws a subset of nearby NKAs into the insoluble complexes it forms, is consistent with a previous observation of NKAs in PrP^{Sc} preparations (Graham et al., 2011) and the complexity of Atp1a1 western blot signals that was observed in the current study in RML-infected but not in mock-infected mouse brains.

Therefore, this study points toward PrP^{C} influencing the activity of NKAs, leading to their activation in healthy cells, and these ion pumps playing a role in the pathobiology of prion diseases

### Example 2: Subtoxic Binding of Cardiac Glycosides to Their Na,K-ATPase Receptors Causes Endolysosomal Cathepsin-Dependent Reduction of Cellular Prion Protein Levels

### A. Methods

### Antibodies

Primary antibodies were sourced as follows: mouse monoclonal (clone 3F4) anti-human PrP antibody (catalog number MAB1562, Millipore, Ontario, Canada; used at 1:1000 dilution), mouse monoclonal IgG1 (clone 464.6) anti-ATP1A1 antibody reactive to various species (catalog number ab7671, Abcam, Ontario, Canada; used at 1:2000 dilution), mouse monoclonal IgG1 (clone C-9) anti-CD109 reactive toward mouse, rat and human (catalog number sc-271085, Santa Cruz Biotechnology, Texas, USA; used at 1:100 dilution), mouse monoclonal IgG1 (clone C4) anti-β-actin antibody reactive against a wide range of species (catalog number sc-47778, Santa Cruz Biotechnology; used at 1:1000 dilution). Horseradish peroxidase-conjugated secondary antibodies against mouse (catalog number 7076S; used at 1:5,000 dilution) and rabbit (catalog number 7074S; used at 1:5,000 dilution) immunoglobulin G were from Cell Signaling Technology and distributed by New England Biolabs, Ontario, Canada.

### Cardiac Glycosides

Ouabain octahydrate (catalog number O3125, Sigma-Aldrich, Ontario, Canada), digoxin (catalog number D6003, Sigma-Aldrich), and oleandrin (catalog number 06069, Sigma-Aldrich) were initially dissolved in water or DMSO, and further diluted in cell culture media to working concentration of 1000× prior to treatments. For treatments of differentiated ReN VM cells spanning several days, CG levels were replenished daily along with a change of half of the cell culture medium.

### Cell Culture

ReN VM cells were maintained in DMEM/F12 (catalog number 11320033, Thermo Fisher Scientific) supplemented with 2% N21-MAX (catalog number AR008, R & D Systems, Minneapolis, MN, USA) or 2% B27(catalog number 17504044, Thermo Fisher Scientific, MA, USA), 20 ng/mL basic fibroblast growth factor (catalog number PHG0261, Thermo Fisher Scientific), 200 ng/mL epidermal growth factor (catalog number RKP01133, Reprokine, Tampa, FL, USA), and 2 ng/mL heparin (catalog number H3149-10KU, Sigma-Aldrich) on Matrigel-coated (catalog number 354230, Corning, Guelph, ON) tissue culture plates at 37° C. with 5% CO₂ as previously described (Kim et al., 2015). ReN VM cells were differentiated into co-cultures of neurons and astrocytes upon removal of growth factors and heparin for at least 7 days, as previously described (Donato et al., 2007). For cryogenic preservation of cell stocks in liquid nitrogen, cells were stored in Recovery Cell Culture Freezing Medium (catalog number 12648010, Thermo Fisher Scientific).

### Immunocytochemistry and Confocal Microscopy

ReN VM cells were cultured as described above. Cells were fixed in 4% formaldehyde for 15 minutes and permeabilized with 0.1% Triton X-100 for 20 minutes. Permeabilized cells were incubated overnight at 4° C. in PBS buffer containing 1% BSA and primary antibody directed against ATP1A1 (catalog number ab7671, Abcam, Waltham, MA; used at 1:200). Following three PBS washes, Alexa-Fluor 633 secondary antibody (catalog number A-21052, Invitrogen, Burlington, ON; used at 1:400) was incubated with cells for 90 minutes at ambient temperature in PBS buffer containing 1% BSA. Cells were washed three times in PBS and mounted onto glass slides using ProLong Gold containing DAPI (catalog number P36934, Invitrogen, Burlington, ON).

### Effects of Digoxin and Oleandrin on Cell Viability, Intracellular Ca²⁺, and ATP Content

ReN VM cells were plated at 16,800 cells/well on a Matrigel-coated black 96-well clear bottom tissue-culture plate. Cells were grown in proliferation media for two days before being differentiated by growth factor withdrawal for 1 week. After 1 week of differentiation, cells were treated with digoxin (at concentrations 0 nM, 1 nM, 3 nM, 10 nM, 30 nM, 100 nM, 300 nM, and 1000 nM) or oleandrin (at concentrations 0 nM, 2.5 nM, 5.0 nM, 7.5 nM, 10 nM, 20 nM, 30 nM, 40 nM, 80 nM, and 100 nM) at 6 biological replicates per concentration with randomized well assignment for each replicate to reduce bias caused by evaporation effects. After 1 week of treatment, cells were analyzed with the following assays:

### Cell Viability Assay Using Calcein-AM (Catalog Number 17783, Sigma-Aldrich)

Cells were washed twice with PBS (catalog number D8537-500ML, Sigma-Aldrich, Oakvill, ON, Canada) and then incubated with 1 μM of calcein-AM in PBS with 3% (w/v) bovine serum albumin (BSA, catalog number ALB001.50, BioShop, Burlington, ON, Canada) for 20 min at 37° C. Cells were then rinsed with PBS to remove calcein-AM before being incubated for another 30 min at 37° C. in just PBS with 3% (w/v) BSA to allow complete de-esterification of AM esters. Excitation/emission signal at 486/516 nm was measured in a microplate reader as an indication of cell viability.

### Cellular ATP Content Assay Using CellTiter-Glo (Catalog Number G7570, Promega, Madison, WI, USA)

Cells were equilibrated to room temperature for 30 min to minimize uneven plate reading due to inconsistent temperature. The CellTiter-Glo® reagent was diluted 1:4 in PBS before use. The volume of diluted CellTiter-Glo® reagent added per well was equal to the volume of differentiation media already present in each well. Plates were then shaken for 2 min on an orbital shaker for 2 min at room temperature to induce cell lysis. Luminescence was recorded with an integration time of 1 s as an indication of cellular ATP content.

### Intracellular Ca²⁺ Content Assay with Fluo-4 AM (Catalog Number F14201, Thermo Fisher Scientific)

Cells were washed twice with PBS and then incubated with 2 μM of Fluo-4® AM in PBS with 3% (w/v) BSA for 20 min at 37° C. Cells were then rinsed with PBS to remove Fluo-4 AM before being incubated for another 30 min at 37° C. in just PBS with 3% (w/v) BSA to allow complete de-esterification of AM esters. Excitation/emission signal at 486/516 nm was measured in a microplate reader as an indication of intracellular Ca²⁺ content.

### CRISPR-Cas9 Mediated Mutagenesis of Human ATP1A1 Rendering Resistance to CG Exposure

ReN VM cells expressing the resistant form of ATP1A1 were generated by transfection using a paired Cas9 nickase design based on two sgRNAs with the protospacer sequences ‘gttectettctgtagcagct’ and ‘gagttctgtaattcagcata’, supplemented with a 200-nt-long single-stranded oligonucleotide (ssODN) repair template (Ultramer Oligos, Eurofin Genomics, Toronto, ON, Canada). The sgRNAs were designed using the CHOP CHOP CRISPR design tool (Labun, K., et al. CHOPCHOP v3: expanding the CRISPR web toolbox beyond genome editing. Nucleic Acids Research (2019).) and selected based on high specificity scores and proximity to the target amino acid residues 118 and 129. The protospacer sequences were inserted into the MLM3636 sgRNA plasmid via site-directed mutagenesis using the Q5 Site-Directed Mutagenesis kit (catalog number E0554S, New England Biolabs, Ipswich, MA). The Cas9 D10A nickase plasmid was generated by site-directed mutagenesis as previously described (Wang et al., 2019). All plasmids were transformed into 5- Competent E. coli (catalog number C2987H, New England Biolabs). All plasmids were purified using the PureLink HiPure Plasmid Filter Maxiprep Kit (catalog number K2100-16, Thermo Fisher Scientific) before transfection.

### Transfection and Selection

ReN VM cells were detached with Accutase (catalog number A1110501, Gibco) and plated at 135,000 cells/well in Matrigel-coated 12-well plates 1 day before transfection in proliferation media without heparin. Cells were then transfected at a ratio of 500 ng total DNA/well: 1 μL/well of TransfX transfection reagent (catalog number ACS-4005, American Tissue Culture, Gaithersburg, Maryland, USA). The ratio of plasmids used was 6 Cas9 nickase: 1 sgRNA: 1 sgRNA: 5 ssODN. 48 h post transfection, cells were treated with 100 nM of ouabain for 7 days to select for cells that have been edited to express the resistant form of ATP1A1. Surviving clones were picked for genomic analysis and cryopreservation.

### Genomic PCR

Genomic DNA was extracted from clones grown on 12-well plates using the PureLink® Genomic Mini Kit (catalog number K182001, Invitrogen). Precise transgene insertion into the target site at the ATP1A1 locus was determined by genomic PCR using 25 ng of genomic DNA amplified for 28 cycles using ‘ttgteggcagctetttggg’ and ‘agtgggagacaaagacggaga’ as the forward and reverse primers, respectively. PCR products were purified with a gel/PCR extraction kit (catalog number DF300, Froggabio, ON, Canada).

### In Vitro Protease Inhibition Studies in ReN Cells

Inhibitors were added 2 hrs before the addition of the CGs. Calpastatin (7316, Clontech, CA, USA), Calpain inhibitor I (A6185, Sigma-Aldrich), MG132 (M7449, Sigma-Aldrich), Lactacystin (L6785, Sigma-Aldrich), Bafilomycin A1 (SML1661, Sigma-Aldrich), Pepstatin A (P5318, Sigma-Aldrich), E64D (E8640, Sigma-Aldrich) were dissolved in DMSO in 1000× stocks. Unless specified otherwise, MG132, E64D and bafilomycin A1 treatments were performed for 24 hrs, calpain inhibitor I for three days and calpastatin for five consecutive days with daily replenishment.

### Protein Extraction and Immunoblotting

Cells were harvested in lysis buffer containing 1% NP40, 150 mM NaCl and 150 mM Tris-HCl (pH 8.3) supplemented with protease and phosphatase inhibitors (11836170001 and 4906837001; Millipore) and subjected to immunoblotting. Briefly, total protein amounts were adjusted across samples using the BCA protein assay kit (23225; Thermo Fisher Scientific). Equal amounts of protein for samples were run on SDS-PAGE gels and transferred to PVDF membranes (IPVH00010, Millipore) and incubated with primary antibody overnight after blocking of unspecific sites in 10% skim milk. Immunoreactive bands were visualized after incubation with the secondary antibodies on X-ray films or a LI-COR Odyssey Fc® digital imaging system (LI-COR Biosciences, NE, USA).

### B. Results

### Sustained Exposure of Differentiated Human Neurons to CGs Reduces PrP^{C} Protein Levels

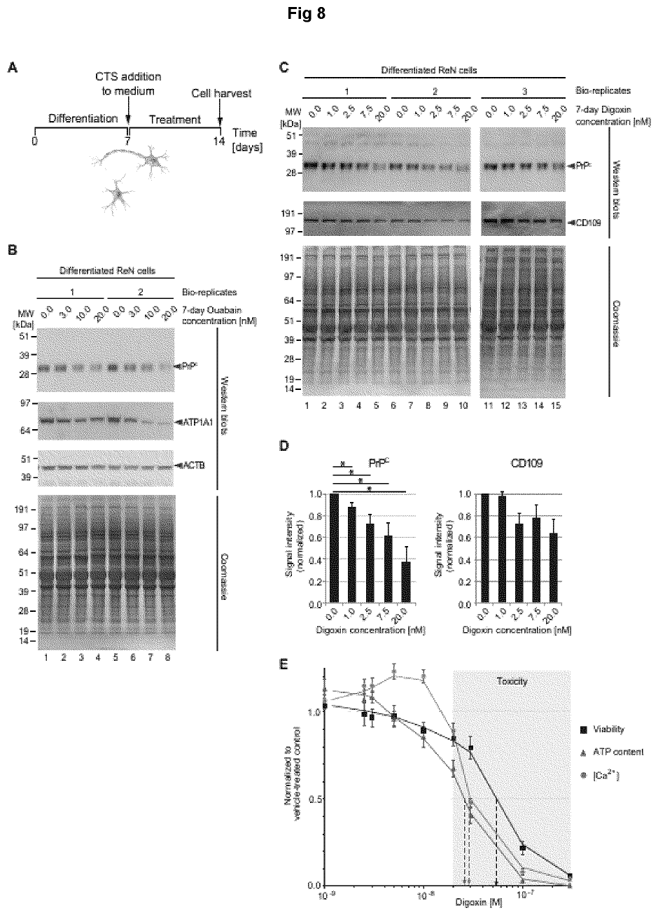

To investigate the effect of CGs on NKA and PrP^{C} levels, ReN VM cells that had been differentiated into co-cultures of human neurons and astroctyes were exposed to a range of nanomolar concentrations of CGs or vehicle solution for a period of one week (FIG. 8A). Western blot analyses of total cellular extracts obtained from these cells documented a ouabain concentration-dependent depletion of PrP^{C} at ouabain levels of 3 nM to 20 nM, i.e., near the previously determined K_{D} for this pump (Lucchesi and Sweadner, 1991). The reduction in steady-state PrP^{C} levels followed the depletion of the ATP1A1 isoform, the most broadly expressed amongst the four NKA α subunit paralogs encoded in the human genome (reviewed in (Clausen et al., 2017)). At the concentrations tested, ouabain did not affect bulk protein levels, which were monitored by Coomassie staining (FIG. 8 B).

Whereas ouabain is primarily used in experimental research due to its low bioavailability, the CG known as digoxin and digitoxin are still widely used in the clinic. Moreover, ouabain dissociates from NKAs after less than two hours (Tobin, 1972 #9312), whereas digoxin-NKA complexes have been shown to take 18 hours to dissociate in vitro (Ford, 1979 #9314). To begin to explore if administration of digoxin can similarly reduce PrP^{C} levels at low nanomolar concentrations, the analyses of the effect of CG on PrP^{C} in the presence of digoxin levels that ranged from 1 nM to 20 nM was repeated. Data collected from three biological replicates for each digoxin concentration demonstrated that digoxin exposure again led to no discernible changes in ReN VM cell bulk protein levels, yet also caused a concentration-dependent reduction in PrP^{C} levels at media concentrations as low as 1-2.5 nM digoxin, a level achieved in serum samples in the clinic (Ehle et al., 2011). The levels of CD109, another GPI-anchored protein that resides in proximity to PrP^{C} in certain cell types (Ghodrati et al., 2018), were also reduced in digoxin-exposed cells, albeit to a lesser extent. While not being bound by theory, this suggeststhat the digoxin-induced removal of NKAs by the cell may not only also engulf the adjacent PrP^{C} molecules but may to some degree extend to other nearby proteins (FIG. 8 C).

Quantitative image analyses of western blot signals revealed steady-state PrP^{C} levels in the presence of 20 nM digoxin to be reduced to approximately 40% of levels observed in vehicle treated cells. Levels of CD109 were reduced approximately half as much as PrP^{C} levels under these conditions to close to 70% of signal intensities observed in vehicles-treated cells (FIG. 8 D).

To assess the cellular health of ReN VM cells treated with digoxin, standard curves were generated that interrogated the percentage of metabolically-active cells (calcein-AM assay), levels of intracellular ATP content (CellTiter-Glo® assay), and intracellular calcium levels (Fluo-4 AM assay) of ReN VM cells cultured in the presence of 1 nM to 300 nM digoxin (FIG. 8 E). These analyses revealed that as intracellular ATP levels dropped in the presence of low nanomolar concentrations of digoxin the levels of cellular calcium increased by approximately 20%. Above 10 nM digoxin levels, all three measures of cellular health dropped. Cellular toxicity—operationally defined as a drop of ATP levels to 50% of those seen in mock-treated control cells—set in at around 25 nM digoxin levels. Half maximum intracellular calcium levels were seen with digoxin levels in the cell medium at 30 nM, and metabolic activity dropped to 50% at around 50 nM digoxin levels.

### The CG-Induced Reduction in PrP^{C} Levels Depends on its Endolysosomal Degradation

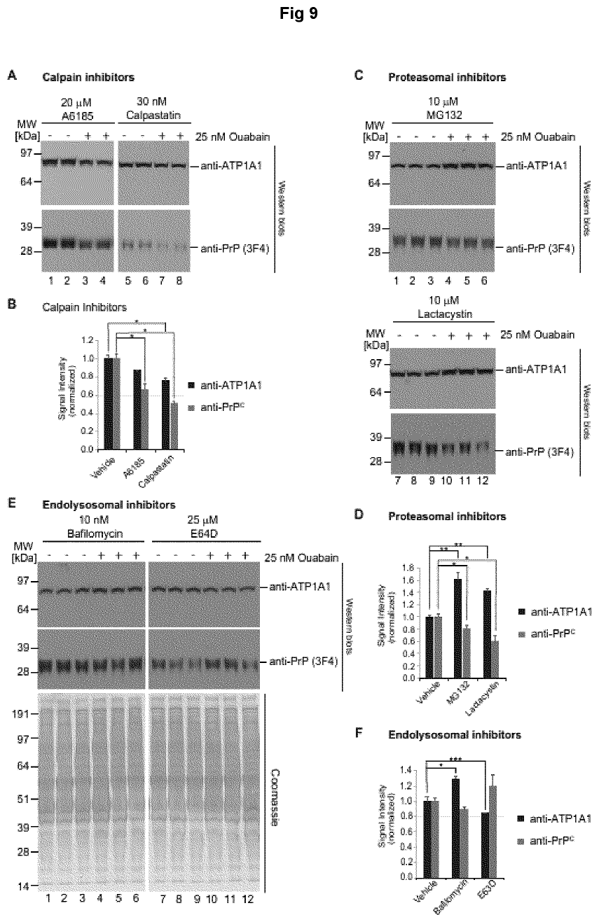

Steady-state levels of PrP^{C} are known to be modulated to varying degrees by calpain-dependent proteolysis (Hachiya et al., 2011), as well as proteasomal (Ma and Lindquist, 2001; Yedidia et al., 2001), and endolysosomal degradation (Ballmer et al., 2017; Shyng et al., 1993), i.e., each of the three dominant protein degradation systems in eukaryotic cells. To begin to understand if any of these systems plays a role in the CG-dependent reduction in steady-state PrP^{C} levels, the ouabain treatment of ReN VM cells in the presence or absence of well-established inhibitors of these degradation pathways was repeated. Because the selectivity of available inhibitors is somewhat crude, two distinct inhibitors were tested separately for each of the three degradation pathways. The design of this experiment was based on the expectation that one of the three degradation pathways may by itself account for the CG-induced reduction in PrP^{C} levels. Consequently, ReN VM cells were again exposed to mock treatment or 25 nM ouabain and it was observed whether the concomitant presence of an inhibitor of one of the three degradation system can block the expected reduction in PrP^{C} levels. When tested in this manner, the presence of two calpain inhibitors (A6185 or calpastatin) did neither prevent the CG-induced reduction in ATP1A1 levels nor the reduction in PrP^{C} levels (FIG. 9A, B). The presence of proteasomal inhibitors (MG132 or lactacystin) also failed to block the CG-dependent reduction in PrP^{C} levels but, intriguingly, caused an increase in ATP1A1 steady-state levels (FIG. 9 C, D). Finally, and in contrast to the aforementioned inhibitors, a blockade of endolysosomal proteolysis, either through inhibiting acidification of the endolysosomal compartment (bafilomycin) or through the addition of a cysteine protease inhibitor that irreversibly modifies the active thiol group present in these proteases (E-64) rescued the CG-induced steady-state level reductions of both ATP1A1 and PrP^{C} (FIG. 9 E, F). Taken together, these experiments indicate that the endolysosomal degradation pathway plays a role in the CG-dependent reduction in steady-state PrP^{C} levels.

### Comparison of Potency of Ouabain and Oleandrin for Reducing PrP^{C} Levels

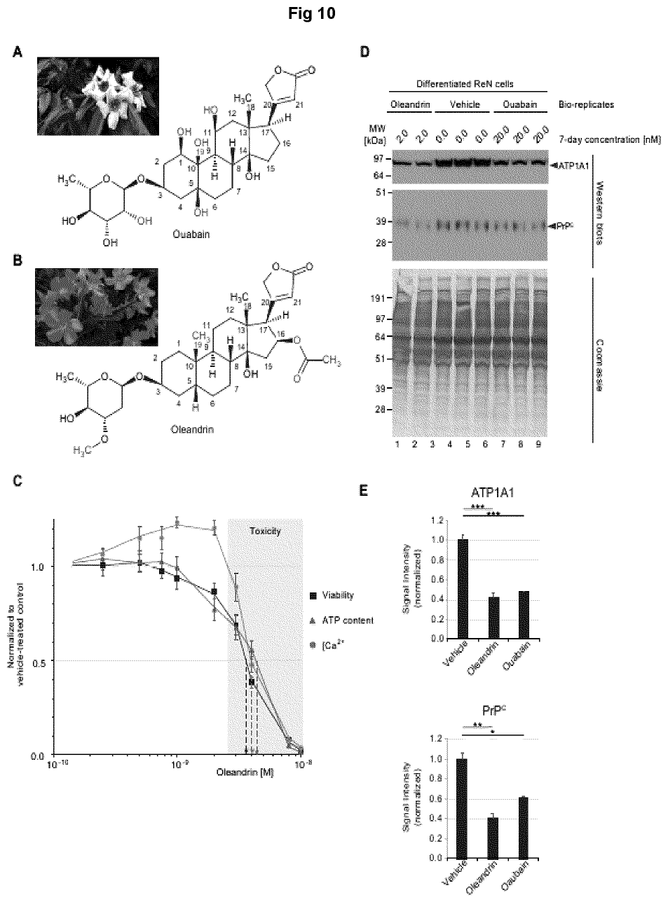

Because neither ouabain nor digoxin are suitable for brain applications due to their relative low membrane permeability (Kuhlmann et al., 1979) and active extrusion from the brain by P-glycoproteins (Batrakova et al., 2001; Mayer et al., 1996; Taskar et al., 2017), alternative CGs known to have higher blood brain barrier (BBB) penetrance were explored. Amongst several CGs that were investigated, oleandrin, a compound that can be extracted from the ornamental plant Nerium oleander, was of interest. This compound has lower hydrogen bonding capacity than ouabain due to the replacement of several hydroxyl groups on its steroid core and glycoside, a feature predicted to improve its brain penetrance (Rankovic, 2017) (FIG. 10A, B).

Exposure to oleandrin levels of up to 2 nM was well tolerated even over prolonged time periods exceeding one week by differentiated ReN VM cells on the basis of their metabolic activity, cellular ATP and Ca²⁺ levels. Further increases to beyond 3.5 nM caused toxicity manifest as 50% or higher reductions in the readout for these measures of cellular health (FIG. 10 C). A side-by-side comparison of oleandrin and ouabain revealed an approximately tenfold higher potency of oleandrin on the basis of achieving a similar reduction in steady-state levels of ATP1A1 and PrP^{C} during a 7-day treatment regime (FIG. 10 D). Influence of cardiotonic steroids on PrP^{C} levels depends on ATP1A1 engagement

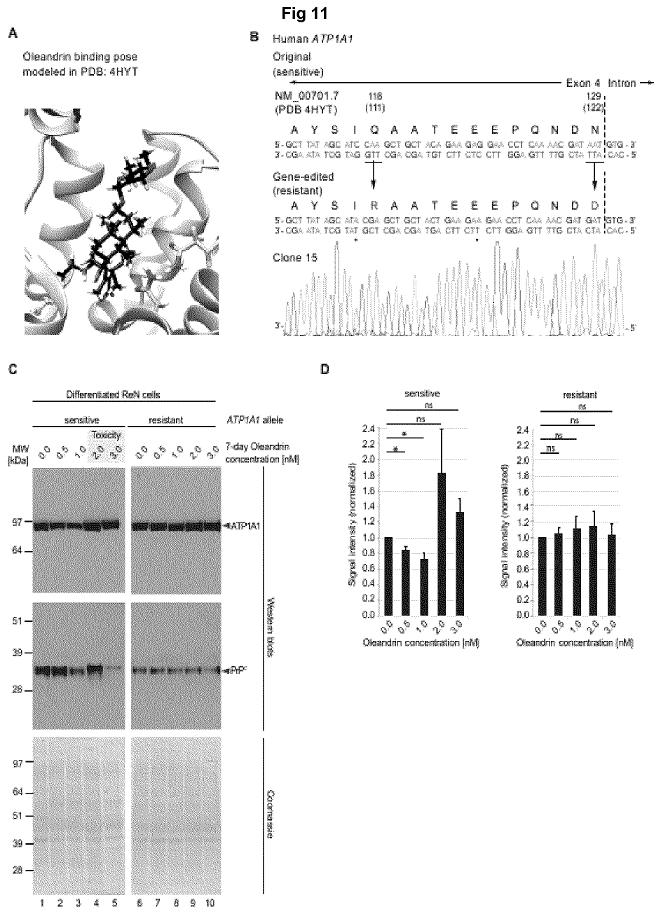

Several scenarios could be invoked regarding the mechanism of action through which cardiotonic steroids impact PrP^{C} homeostasis in a manner that might trigger its endolysosomal degradation, including the possibility that cholesterol-like features of molecules in this compound class can disrupt cholesterol-rich raft membrane domains that PrP^{C} resides in. Moreover, if the diminution of PrP^{C} relies on an engagement of CG with their Na,K-ATPase receptor targets, it would be instructive to learn, which of the four human subunit isoforms mediates this effect. Although the aforementioned experiments pointed toward ATP1A1 as the most likely mediator of the CG-induced PrP^{C} reduction, none of them proved this mechanism of action. To address these questions, precise gene editing using CRISPR-Cas9 technology was applied to engineer ReN VM cells that express a mutated CG-resistant derivative of the ATP1A1 isoform. The design of this gene editing step capitalized on prior work by others, which had shown that a glutamine (residue 111) and an asparagine (residue 122) residue in the CG binding pocket of the ATP1A1 (amino acid numbering as for the PDB entry 4HYT) are involved in binding (FIG. 11A). Exchanging these two residues to arginine and aspartic acid, respectively, amino acids present in these positions in several rodent ATP1A1 orthologs, is known to lower the CG binding affinity of this isoform approximately 1000-fold (Price and Lingrel, 1988; Price et al., 1990) and is the reason for dramatically higher tolerances of rodents toward CG exposure (Wansapura et al., 2011). Using a paired Cas9 nickase strategy, ReN VM cells were transfected with gRNAs, which caused staggered cuts near the genomic ATP1A1 target site that were subsequently repaired using a 200-nucleotide-long single-stranded oligonucleotide (ssODN) repair template carrying the intended point mutations. Several ReN VM cell clones were obtained, which were sequence-validated to be homozygous for the mutated human CG-resistant ATP1A1 isoform (FIG. 11 B). Next, the response of wild-type CG-sensitive and mutated CG-resistant ReN VM cells to a range of low nanomolar oleandrin concentrations was compared. In three biological replicates of this experiment, a consistent response of wild-type ReN VM cells was observed, which was characterized by an initial decline of ATP1A1 levels at oleandrin concentrations of 0.5 and 1 nM, followed by a sharp increase in steady-state pump levels at around 2 nM, which tended to get weaker at higher more toxic concentrations (FIG. 11 C, D). When the same samples were probed with PrPc-directed antibodies, essentially the same trend was observed. However, rather than leading to a mere rebound of PrP^{C} steady-state levels at 2 and 3 nM oleandrin concentrations, the PrP^{C} signal also migrated reproducibly slower at these borderline toxic concentrations of this cardiotonic steroid. In cells that express the mutant CG-resistant ATP1A1 protein, neither the changes in ATP1A1 nor in PrP^{C} levels were observed. Taken together, these data revealed the formation of a high-affinity CG ligand-ATP1A1 receptor complex to be the mechanism of action through which CG exposure of human co-cultures of neurons and astrocytes triggers the endolysosomal degradation of PrP^{C} that translates into reduced steady state levels of this protein.

### C. Discussion

This study showed that a CG-dependent reduction in PrP^{C} levels occurs in a CG concentration-dependent manner when cells are exposed to members of this compound class. Rather than affecting all human paralogs of NKA α subunits equally, it was demonstrated that this outcome primarily impacts the levels of ATP1A1 and that a high-affinity complex of the CG bound to its cognate ATP1A1 binding pocket reduces the levels of both ATP1A1 and PrP^{C}.

Although the levels of a vast majority of cellular proteins were not affected when cells were exposed to CG, PrP^{C} is unlikely to be the only protein outside of cognate NKAs comprising ATP1A1 that exhibited reduced steady-state levels in the presence of CGs. Our observation that CD109, which had been previously observed to reside in spatial proximity to PrP^{C} (Ghodrati et al., 2018), was also reduced in its steady-state levels as a consequence of the formation of this primary ligand-receptor complex, supports this conclusion. It may be most plausible to assume that upon CG engagement a patch of membrane that harbors the targeted NKA, along with its closest molecular neighbors, come along for the ride of cellular internalization. Once internalized, individual proteins within this patch of membrane may experience distinct fates. This interpretation may also provide an alternative mechanistic explanation for a previously reported reduction in TGF-β receptor-2 levels in ouabain treated human lung fibroblasts, which the authors had attributed to a CG-induced reduction in mRNA levels coding for this growth factor receptor (La et al., 2016).

The study of NKAs is fraught with complexities: Their investigation in human and rodent cells has to account for the existence of four α and three β subunit paralogs that make up the main building blocks of these heterodimeric protein complexes (Blanco, 2005) (a fourth β subunit, ATP1B4, has in vertebrates acquired a novel function in the inner nuclear membrane (Korneenko, 2016). Additional combinatorial complexity derives from observation that individual α and β isoforms may exhibit preferential associations (Tokhtaeva, 2012)(Habeck, 2016) rather than fixed pairings (Schmalzing, 1997). Moreover, individual cells, including the astrocytes and neurons of the human ReN VM cell paradigm employed in this study, have distinct expression profiles of a subunits (Azarias, 2013)(Cameron, 1994 #6262)(Martin-Vasallo, 2000) and these profiles shift as the cells differentiate (Lecuona, 1996).

The present results supports the existence of collaboration and built-in redundancies amongst subunit paralogs. Consistent with this interpretation, the CG-mediated reduction in ATP1A1 levels was paralleled by a reduction also of ATP1A2 and an increase in ATP1A3 at slightly higher CG concentrations. It was also observed that the increase in CG concentrations toward borderline toxic levels generated a rebound in the levels of ATP1A1. One plausible explanation for this observation is the existence of a cellular response that senses and compensates for the diminution of NKS activity by increasing their production and/or slowing their degradation.

Because of its potential therapeutic implications, the observation that PrP^{C} levels in a human co-culture model of neurons and astrocytes can be reduced by more than 50% when cells are exposed to non-toxic levels of CG, a class of compounds that is exceedingly well-characterized in its clinical use following decades of research is significant. Several members of this compound class are well-known poisons to a wide range of species due to their exquisite potency and their targeting of a critical pump held responsible for maintaining the electrochemical gradient in eukaryotic cells. The upside of this potency is that CG can achieve reductions in PrP^{C} levels at low nanomolar concentrations. Contrast this result to previous data from a high-throughput screen of 1,280 compounds that were derived from the US Drug Collection and reported micromolar effective concentrations for the most favorable compounds, Tacrolimus and Acetimole (Karapetyan et al., 2013). Another systematic screen of a large library of 44,578 compounds led to the identification of 32 compounds that reduced PrP^{C} levels at 65 nM to 4.1 μM concentrations without killing the cells (Silber et al., 2014).

The best-understood FDA-approved CGs have a narrow therapeutic window and reach lower concentrations in the brain than in peripheral tissue, which is acceptable for the treatment of heart defects but a hindrance for possible neurodegenerative indications. However, these challenges are not insurmountable as more about the constraints that limit access of CGs to the brain are understood. For example, a subset of CGs are actively removed from the brain through the P-glycoprotein (P-gp), which offers an opportunity to concomitantly restrict the activity of this antiporter system. In fact, studies in mice have established that brain levels of digoxin can be more than tenfold increased in the presence of elacridar, a specific inhibitor of P-gp (Taskar et al., 2017), or upon knockout of the two P-gp genes conferring multidrug resistance (mdr1a and mdr1b) in mice (Schinkel et al., 1997).

Thus, once a well-established ‘druggability’ of CGs is combined with insights into the P-gp binding properties and BBB penetrance of a subset of these compounds, a workable path toward a CG-based treatment of prion diseases that targets PrP^{C} levels will come into focus.

### Example 3: Identification of Exemplary Cardiac Glycosides Exhibiting Favorable Brain Bioavailability and Potency for Reducing Levels of the Cellular Prion Protein

### A. Methods

### Sequence Alignment and Identification of Residues Lining CG Binding Pocket

Sequence identity assessments made use of available sequences for S. scrofa ATP1A1 (P05024), H. sapiens ATP1A1 (P05023), ATP1A2 (P50993), and ATP1A3 (P13637) using the Basic Local Alignment Search Tool (BLAST) and the following settings: E-threshold: 0.001; matrix: BLOSUM62, and allowing both the filtering of low complexity regions and the introduction of gaps). The multiple sequence alignment algorithm Clustal O® (Version 1.2.4) was used to determine homologous residues lining the CG binding pocket.

### Structural Modeling and Assessment of Docking and BBB Penetrance Scores

Because no crystal structure of a human NKA is available, a homology model of human a subunits was built on available structures for pig NKAs. Modeling focused on PDB entry 4HYT because it was co-crystallized with a cardenolide CG, namely ouabain+Mg2+ ion, at a resolution of 3.4 Å. The availability of another pig NKA structure, co-crystallized with a bufadienolide, namely bufalin+2K+ ions, at a similar resolution (4RES) was initially compared to learn about the degree to which CGs with five- versus six-membered lactone rings and different conformational states of the NKS complex affect the CG binding pose. Based on primary sequence comparisons it was hypothesized that human NKA harboring ATP1A3 bound to a cardenolide show the same macro 3D structure as pig NKA. Co-crystal of human ATP1A3 bound to ouabain or oleandrin were modeled using soft minimization of ouabain geometries and amino acid residues within 5 Å around it. Binding free energies were approximated using GBSA method. Docking studies within the human ATP1A3 model reproduced the binding mode of ouabain reported in the literature and hypothesized a highly similar binding pose for oleandrin.

### Design of Chemically Accessible Oleandrin Derivatives

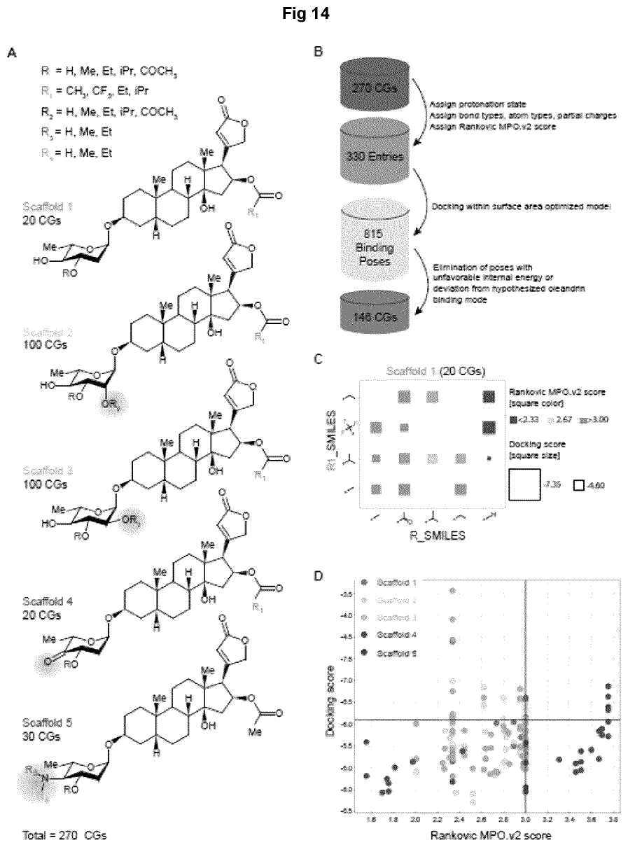

Scaffolds 1-4 depicted in FIG. 14 were design based on their potential synthetic availability (1-5 step sequences) from the commercially available natural products oleandrin and gitoxin. It was envisioned that compounds depicted by scaffold 1 would be derived from oleandrin by acyl group modification at the C16 position. The compounds denoted by scaffolds 2 and 3 are derivatives of gitoxin that would be obtained through the introduction of the C16 acyl group followed by a deglycosylation/reglycosylation sequence. The compounds denoted by scaffolds 4 and 5 represent derivatives of oleandrin, and it was envisioned that scaffold 4 would be synthesized through a known oxidation of the 4′ position (Zhang et al., 2020; Czernecki et al., 1985) followed by the C16 acyl group adjustment. The scaffold 5 would in turn be derived from scaffold 4 through a known reductive amination (Chen et al., 2016). All compounds were assessed and ranked on the basis of their predicted Rankovic MPO.v2 and in silico docking scores.

### Synthesis of Shortlisted Oleandrin-Derivative

The derivative exemplary compound I-3 was synthesized by using well established protocol for the PCC oxidation of oleandrin (Chen, H. 2016, and Zhang, Y. et al. 2020). Following the published purification protocols, the synthetic material was found to be >95% pure and was used as such for the subsequent biological studies.

### Characterization of Exemplary Compound I-3 by Mass Spectrometry

A 780 μM DMSO solution of exemplary compound I-3 was stored at 37° C. and sampled after 1, 3, 7 and 14 days. Each sample was frozen at collection. Upon thawing, the samples were spiked with deuterated oleandrin in DMSO then dried in a centrifugal concentrator. The remaining solids were dissolved in 0.1% formic acid in 1:1 methanol to water, making the concentration of deuterated oleandrin 2 μM and that of exemplary compound I-3 equivalent to 10 μM.

The solutions were infused at 2 μl/minute through a heated electrospray ionization source coupled to an Oribtrap Fusion® mass spectrometer. Mass spectra were collected on the orbitrap at a nominal resolution of 60,000 with the automatic gain control target held at 4.0e5, and 30 seconds of data from each sample were averaged for quantification.

### Tritium-Based Comparison of Bioavailability of Oleandrin and Exemplary Compound I-3

All work with mice was approved by the University Hospital Network Animal Care Committee.

Tritiated exemplary compound I-3 with a specific activity of 1.6 Ci/mmol and a concentration of 1.0 mCi/mL was obtained through a customized radiolabeling request (Moravek Inc., Brea, California, USA). To minimize rapid back exchange of tritium with available protons, the radiolabeled product was overwhelmed with non-labeled water and organic solvent three times. This procedure achieved an amount of exchangeable tritium <0.1%. The certificate of analysis attested to a 100% radiochemical purity on the basis that 99.68% of radiolabeled material co-eluted with the unlabeled reference standard on a Zorbax SX® 4.6×250 mm column (Agilent) using a mobile phase composed of 26% methanol, 26% acetonitrile, 48% water and 0.1% TFA (v/v).

### Treatment of ReN VM Cells with Exemplary Compound I-3

ReNcell® VM Human Neural Progenitor Cells (SCC008, Millipore) were grown in their undifferentiated form on 20 ug/mL Cultrex® reduced growth factor basement membrane (3433, R&D systems) in DMEM/F1®2 (11320033; Thermo Fisher Scientific) based media with 0.22 filter sterilized 20 ng/mL human basic fibroblast growth factor (bFgF)(RKP09038, Reprokine), 20 ng/mL human recombinant epidermal growth factor (EGF)(RKP01133, Reprokine), 10 units/mL heparin Na+ salt (Sigma, H3149-10KU, cell culture-tested), 2% (1×) v/v B-27 supplement (50×) (17504044, Gibco), 1% v/v Glutamax® (35050061, Gibco) and 1% v/v non-essential amino acids (11140050, Gibco). Differentiation into a co-culture of neurons, astrocytes, and oligodendrocytes was initiated with the removal of growth factors and heparin salt from media. Cells were differentiated for 7 days with replacement of media every 2 days. Treatment was initiated on day 8 of differentiation with the addition of nanomolar concentrations of exemplary compound I-3 or oleandrin ranging between 4 nM to 40 nM to the differentiation media. Treatment continued for 7 days with the daily removal of 50% of media and its replenishment with media containing the appropriate concentration of drug. Vehicle treated cells received the same concentration of DMSO as drug treated cells.

### Antibodies

Immunoblotting analyses of PrP^{C} relied on the monoclonal 3F4 antibody (MAB1562, Millipore) which recognizes the 109-112 amino acid epitope of human PrP. Antibodies against the Na+/K+ ATPase subunits consisted of anti-ATP1A1 (ab7671, abcam), anti-ATP1A2 (AB9094-1), anti-ATP1A3 (MA3-915, Thermo Fisher Scientific) and anti-ATP1B1 (GTX113390, GeneTex). Neuronal and astrocytic markers made use of the NeuN antibody (EPR12763, abcam) and GFAP antibody (131-17719, Thermo Fisher Scientific).

### Western Blot Analyses

Cells were lysed in ice cold buffer containing 1% NP40, 150 mM Tris-HCL (pH 8.3) and 150 mM NaCl supplemented with a protease inhibitor cocktail (11836170001; Roche) and phosSTOP phosphatase inhibitor cocktail (4906845001, Millipore). Insoluble cellular debris was removed following a 5 minute slow speed centrifugation step (2000 g), followed by a 15 minute fast speed centrifugation (16000 g). Protein concentrations of the supernatants were determined via the bicinchoninic acid assay (BCA) using the Pierce BCA Protein Assay Kit (23225, Thermo Fisher Scientific). Protein levels were equally adjusted using the lysis buffer used for cell lysis. Samples for immunoblot analysis were denatured and reduced in 1× Bolt LDS sample buffer (B0007, Thermo Fisher Scientific) and 2.5% Beta-mercaptoethanol (M6250, Sigma) and were heated at 95° C. for 10 minutes. Proteins were separated by SDS-PAGE on a 10% Bolt Bis-Tris gel (NW00105BOX, Thermo Fisher Scientific) then transferred to a 0.45 micron PVDF membrane (IPVH00010, Millipore) and blocked in 10% skimmed milk (SKI400, BioShop) for 1 hour at room temperature. Membranes were then incubated in primary antibodies diluted in 5% skimmed milk and left overnight at 4° C. with gentle rocking. Following day, membranes were washed three times in 1× tris buffered saline with 0.08% Tween20® (TWN508, BioShop) (TBST) then incubated in the corresponding HRP-conjugated secondary antibodies diluted in 5% skimmed milk and left on rocker for 1 hour at room temperature. Membranes were again washed three times in 1× TBST then incubated with enhanced chemiluminescent (ECL) reagent (GERPN2232, GE HealthCare) for 1 minute. Membranes were exposed to autoradiography film (CLMS810, Clonex®) and developed via a film developer.

### B. Results

### Study Design

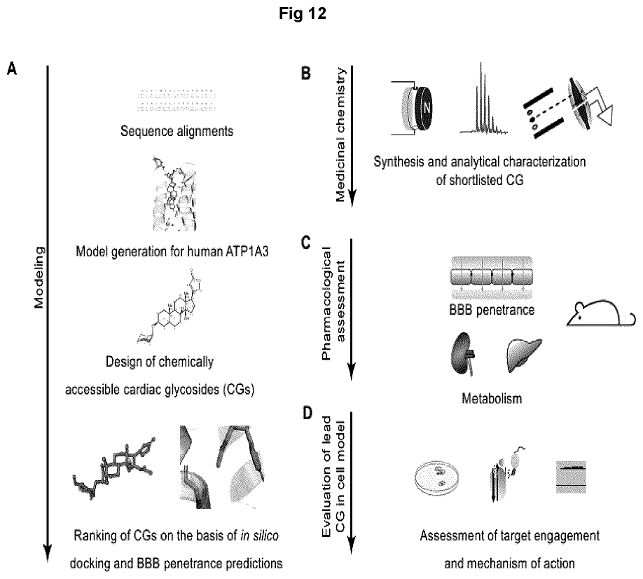

A vast majority of commercially available CGs exhibit poor brain bioavailability, either because they are too hydrophilic or are actively extruded from the brain (Mayer et al., 1996; Turan et al., 2006; Zhou et al., 2019). To the best of the Applicant's knowledge, no large libraries of less investigated CGs are commercially available. The de novo synthesis of CGs is challenging and requires dozens of steps (Zhang et al., 2008; Reddy et al., 2009; Renata, Zhou & Baran, 2013; Mukai et al., 2015; Khatri et al., 2019). Therefore, a small number of compounds were shortlisted on the basis that they are predicted to have high blood-brain-barrier penetrance and are chemically accessible with high purity and yield through a small number of derivatization steps. Next these compounds were in vivo evaluated for their tissue distribution. Finally, their ability to reduce steady-state levels or PrP^{C} was determined in vitro in human co-cultures of neurons and astrocytes and in vivo in mice (FIG. 12).

### In Silico Modeling of Oleandrin Binding to Human NKA α Subunit

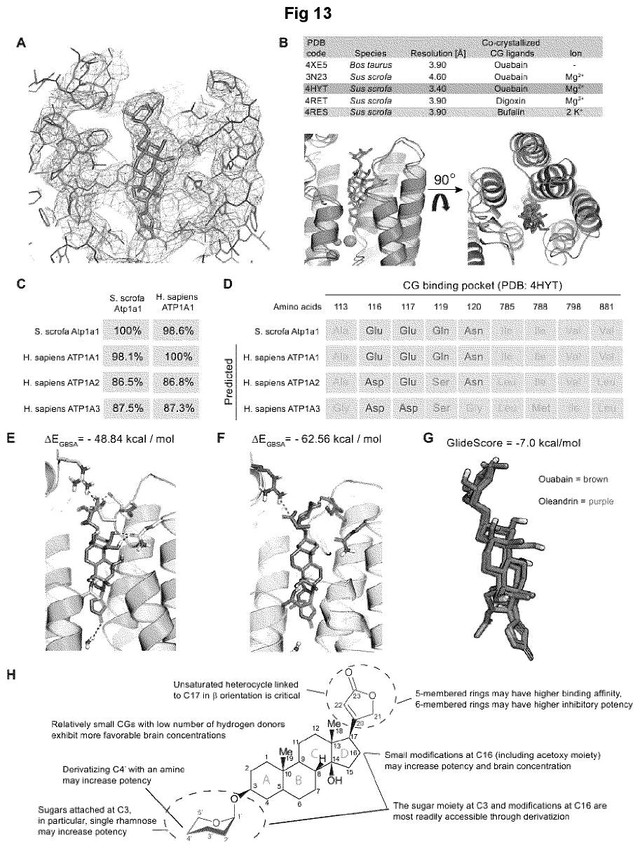

To be able to compare in silico the NKA binding properties of CGs for which no high resolution binding data are available, a suitable model was first generated and evaluated. Several high resolution models have been published for porcine NKAs that show CGs to occupy a binding pocket that can be accessed from the extracellular space and is molded from amino acid residues contributed by the a subunit (FIG. 13A). Different CGs show highly conserved binding to this site even in NKA structures captured in different ion binding states (FIG. 13 B). To assess the merit of building any human NKA model for the intended objective on the basis of the porcine structures, the sequence similarity between human and porcine NKA α subunits (FIG. 13 C) was compared, which indicated almost perfect sequence identity amongst ATP1A1 orthologs and showed that even human ATP1A2 and ATP1A3 paralogs share 87% sequence identity to porcine Atp1A1. The alignment of amino acid residues known to line the binding pocket in porcine Atp1A1 with the homologous human residues, predicted that key properties of the binding pocket are likely maintained in human a subunits, including the existence of previously described hydrophobic and charged internal faces (FIG. 13 D). ATP1A3 is predominantly expressed in brain neurons, and therefore a model needed to be built also for this paralog. Docking algorithms were applied to predict binding poses for oleandrin within this model and to evaluate the likelihood of binding poses to exist naturally on the basis of the free binding energies (ΔG) associated with them. Two main methods were used to evaluate the proposed ligand-protein complexes, the generalized Born model (FIG. 13 E) and the slower but more accurate Molecular Mechanics-Poisson Boltzmann Surface Area (MM-PBSA) method, which computes the free binding energy by subtracting the free energy of the docked ligand-receptor complex from the free energies of its separate components (Poli et al., 2020) (FIG. 13 F). The latter revealed a free binding energy of −62.56 kcal/mol for a surface area-optimized binding pose of oleandrin within the canonical CG binding pocket of ATP1A3. The subsequent comparison of this binding pose revealed exquisite structural alignment with the experimentally observed ouabain binding pose, validating it to be a highly plausible naturally occurring pose (FIG. 13 G). Considering features that may increase the brain bioavailability of CGs (FIG. 13 H) directed attention toward the CG sugar moiety and opportunities to derivatize C16 within the steroid core.

### Evaluation of Chemically Accessible Oleandrin Derivatives on the Basis of Binding Score and Brain Bioavailability

Subsequent efforts focused on 270 CGs that were thought to be chemically accessible through combinatorial derivatization of five CG Scaffolds (FIG. 14A). Molecules in this compound library were put through a virtual screen (VS) for those that might have good binding characteristics and improved brain bioavailability. Initially, compounds were evaluated on the basis of physicochemical properties that determine the propensity for brain penetrance, which can be computed as a multi-parameter optimized (Version 2) score (Rankovic, 2017) (FIG. 14 B). Once the protonation state and partial charges were considered, the number of VS entries increased to 330. Using the aforementioned predicted ATP1A3 model and the MM-PBSA docking method, these entries gave rise to 815 binding poses, a majority of which could be dismissed due to unfavorable internal energies or docking to a site that deviated from the canonical CG binding site. Data obtained when evaluating the 20 CG permutations defined in Scaffold 1 illustrate a typical result (FIG. 14 C). In this case, six of the 20 CGs were rejected due to excessive internal energies or a binding pose that deviated fundamentally from the canonical CG binding pose. Rankovic MPO.v2 scores assigned to the remaining 14 CGs associated with this scaffold ranged between 2.33 and 3.00 and therefore provided no improvement of predicted brain penetrance relative to oleandrin. Finally, Glide docking scores for these CGs ranged from −4.60 to −7.35, indicating that a subset of these CGs were predicted to have improved free binding energies, relative to oleandrin. Overall, 146 CGs passed the aforementioned filters. Amongst these, Scaffold 4-derived CGs stood out because they exhibited the widest breath of Rankovic MPO.v2 scores and were consistently predicted to improve binding to ATP1A3. In contrast, all Scaffold 5-derived were predicted to exhibit improved brain penetrance but only a subset of these exhibited improved free binding energy relative to oleandrin (FIG. 14 D)

### Synthesis of Lead Compound with Favorable Characteristics

Results from the VS recommended a small number of molecules for further analyses on the basis that they were predicted to exhibit comparable or improved binding to human ATP1A3 and to pass the BBB better than the oleandrin reference compound, which was computed to have a Glide docking score of −6.4 and an intermediate Rankovic MPO.v2 score of 3.0 (FIG. 15A). More specifically, eight molecules, exemplary compounds I-1 to I-8 (FIG. 15 B, C) were shortlisted, prioritizing exemplary compound I-3 (4′-dehydro-oleandrin) for this study because (i) it can be easily obtained through derivatization of oleandrin (Chen et al., 2016), (ii) constitutes an intermediate for the synthesis of KDC201-KDC206, and (iii) was computed to have a high Rankovic MPO.v2 score of 3.75. Although not pursued here, replacing the C4′ hydroxyl with an amine is expected to further increase binding to ATP1A3 (exemplary compounds I-7) (FIG. 15D). Similarly, introducing a C2′ methoxy group (KDC201) or a C3′ isopropyl moiety (exemplary compounds I-5) within the exemplary compound I-3 sugar were predicted to further improve binding to ATP1A3 (FIG. 15 E). Finally, a gain in the Glide docking score was consistently attained when hydrogen atoms were replaced within the C16 acetoxy group of the oleandrigenin core with fluorine atoms (exemplary compounds I-2, I-4, I-6 and I-8) (FIG. 15 F). To move from in silico to biochemical analyses, exemplary compound I-3 was obtained through oxidization of oleandrin in dichloromethane followed by HPLC clean-up as a white solid with >95% purity (assessed by ¹H NMR). A stably tritiated version of this compound was obtained commercially through a customized labeling request.

### Analysis of Brain Bioavailability of Lead Compound

To assess the bioavailability of exemplary compound I-3 in a model whose NKAs respond similarly to CGs as their human orthologs, wild-type rodents would not be a good choice, because they have long been known to exhibit more than hundred-fold lower sensitivity toward a large range of CGs than most other mammals (Repke, Est & Portius, 1965; Wallick et al., 1980; Gupta, Chopra & Stetsko, 1986), a difference that has since been attributed to specific differences in Atp1A1 amino acids lining the CG binding pocket. Rather than moving to larger mammals, whose Atp1A1-genes may be more similar to the human ortholog in this regard, studies with Atp1A1 gene-edited mice engineered to carry two point mutations in the first extracellular loop, namely amino acid exchanges Q111R and N122D (amino acid numbering as for the PDB entry 4HYT) coded by the Atp1A1 gene (Atp1a1^{S/S}) were pursued (Price, Rice & Lingrel, 1990). These point mutations are understood to sensitize this a subunit toward CG binding by making it more “human-like” (Dostanic-Larson et al., 2005; Dostanic et al., 2004). At selected time intervals following the subcutaneous injection of Atp1A1^{S/S} mice with tritiated comparative compound oleandrin or exemplary compound I-3 the mice were sacrificed, transcardiac perfused with phosphate-buffered saline and radioisotope levels determined in their brains, hearts, kidneys and livers (FIG. 16A). These analyses revealed as early as 2 hrs after the subcutaneous injection a rapid increase in comparative compound oleandrin in the heart, kidney and liver that was not observed in the exemplary compound I-3 mice. The latter mice reached their highest exemplary compound I-3 levels in these organs after 4 hrs.

Remarkably, whereas comparative compound oleandrin levels were lowest in the brain, radiolabeled exemplary compound I-3 signals in the brain levels exceeded its levels in the heart at all time points tested, and even surpassed kidney and liver levels at the 24 hour time point, when it was determined to have accumulated to a 38.8 nM concentration in the brain. The subsequent analysis of five mice per cohort that were again subcutaneously injected with either tritiated comparative compound oleandrin or tritiated exemplary compound I-3 validated robustly higher brain levels of exemplary compound I-3 than those achieved with comparative compound oleandrin (FIG. 16 B).This analysis also confirmed that brain exemplary compound I-3 levels consistently exceeded heart levels for this compound 24 hours after subcutaneous injection.

In Vitro Assessment of Lead Compound Potency in Human Co-Cultures of Neurons and Astrocytes

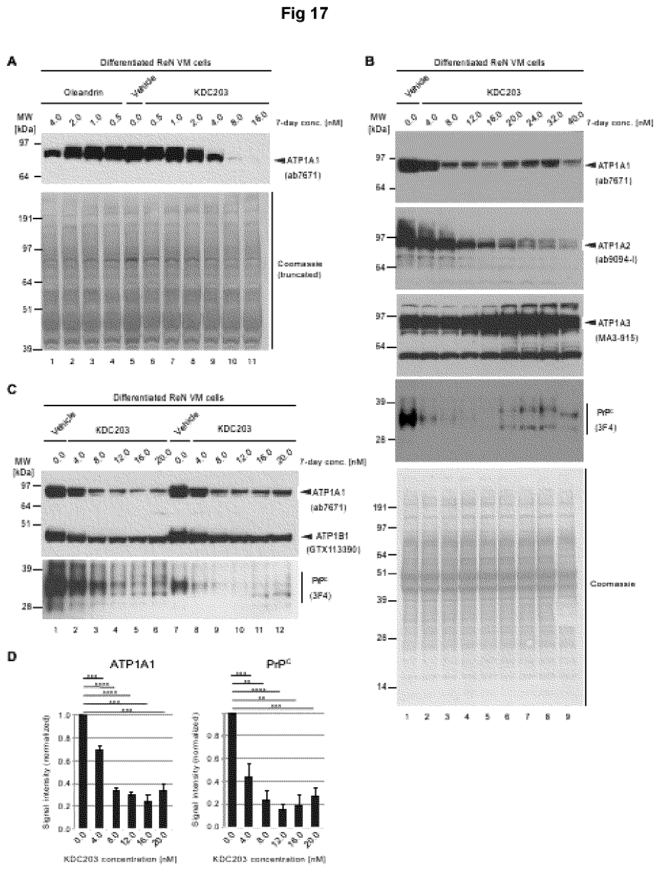

Because the Glide binding scores for comparative compound oleandrin and exemplary compound I-3 were predicted to be identical at −6.40, it was thought that both compounds may have similar potency in an assay that relies on the engagement of these compounds with their cognate NKA α subunit binding site. To evaluate this characteristic experimentally in a relevant model, differentiated co-cultures of human neurons and astrocytes (ReN VM cells) were exposed for a duration of one week to low nanomolar levels of comparative compound oleandrin or exemplary compound I-3. Following cell lysis, steady-state ATP1A1 levels were assessed by western blot analysis to determine if the ligand-receptor interactions had caused the previously observed reduction in ATP1A1 levels. This analysis established that exemplary compound I-3 had indeed similar potency as comparative compound oleandrin on the basis that both drugs achieved a similar reduction in ATP1A1 levels when added at 4 nM concentration to the cell culture medium (FIG. 17A). However, whereas comparative compound oleandrin is toxic at concentrations exceeding 4 nM, exemplary compound I-3 was tolerated by the cells at concentrations up to 30 nM. Surprisingly, exposure of ReN VM cells for one week to 8 or 16 nM levels of this compound led to additional reductions in ATP1A1 levels.

In light of the role of NKAs for maintaining the electrochemical gradient in all eukaryotic cells, the striking exemplary compound I-3-dependent diminution in the steady-state levels of ATP1A1 raised the question whether any of the other NKA α subunits compensated for reduced ATP1A1 levels. To address this point, a repeat exemplary compound I-3 treatment of differentiated ReN VM cells was undertaken with up to 40 nM concentrations of the compound, and cellular extracts were analyzed for all three NKA a subunits expected to be expressed in these cells (FIG. 17 B). Western blot results from this experiment corroborated the robust exemplary compound I-3-induced reduction in steady-state levels of ATP1A1 but also revealed that this effect of the compound extends to ATP1A2 levels. More specifically, ATP1A1 levels were diminished in cells treated with exemplary compound I-3 up to 16 nM levels but rebound slightly when exemplary compound I-3 levels were further increased. In contrast, steady-state ATP1A2 levels declined as levels of exemplary compound I-3 were increased, with no sign of a rebound in the higher concentration range. Intriguingly, ATP1A3 levels stayed unchanged in the presence of up to 8 nM exemplary compound I-3 levels, then increased and reached a maximum in the presence of 24-32 nM exemplary compound I-3 before declining again at 40 nM exemplary compound I-3 levels. When the same cells were probed with an antibody that detects human PrP^{C}, its steady-state levels were revealed to be dramatically reduced in cells exposed to 4-16 nM exemplary compound I-3. Interestingly, in cells exposed to exemplary compound I-3 concentrations of 20-32 nM, a rebound in the intensity of PrP^{C} signals was documented. However, the PrP^{C} signals observed under these circumstances were split into two dominant signals that migrated with apparent molecular weights of 30 and 35 kDa, in contrast to the dominant PrP^{C} signal detected at 32 kDa in naïve cells. Importantly, the exemplary compound I-3 exposure of the cells did not affect bulk protein levels as these were constant for all concentrations tested. Further analysis established that the reduction in the levels of NKA α subunits is paralleled by a lesser, yet also pronounced reduction in the levels of the NKA β subunit (FIG. 17 C). The quantitation of intensities of western blot signals revealed that a maximum 84% reduction in steady-state PrP^{C} levels was attained when ReN VM cells were exposed for one week to 12 nM concentrations of exemplary compound I-3 (FIG. 17 D).

### Replacement of Amino Acids within ATP1A1 Binding Site for CGs Abolishes PrP^{C} Reduction

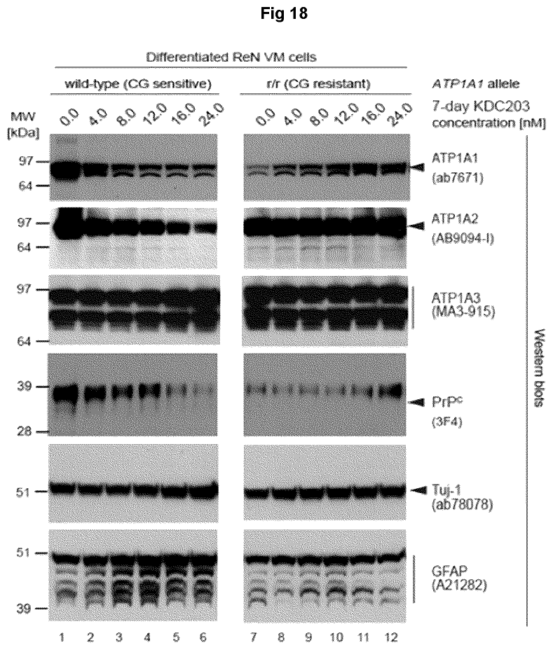

Intrigued by the observation that levels of ATP1A1 and ATP1A2 correlated inversely yet ATP1A3 correlated directly with exemplary compound I-3 levels in its non-toxic concentration range, if the levels of these α subunits were independently or interdependently affected by exemplary compound I-3 was considered. By rendering the human ATP1A1 allele resistant to high-affinity CG docking through mutation of the aforementioned residues to the corresponding amino acids present in mice (ATP1A1 r/r) (Price & Lingrel, 1988), it has been previously observed that the CG-dependent reduction in the steady-state ATP1A1 levels were rescued (Applicant's unpublished observation). This observation emphasized the need for ligand-receptor interaction to achieve this outcome, as opposed to other less defined mechanisms of interaction. Here this ReN VM-derived ATP1A1 r/r cell model was used to investigate whether the exemplary compound I-3-induced changes to ATP1A2 and ATP1A3 are triggered by this CG occupying the respective cognate binding sites in these paralogs or are influenced by exemplary compound I-3 docking to its cognate binding site on ATP1A1. Remarkably, abrogating binding of exemplary compound I-3 to ATP1A1 r/r not only precluded the changes in steady-state levels of ATP1A1 but also prevented changes to the steady-state levels of ATP1A2 and ATP1A3 (FIG. 18A). Moreover, these experiments validated the hypothesis that the exemplary compound I-3-mediated reduction in steady-state levels of PrP^{C} depends strictly on this CG forming a ligand receptor complex with ATP1A1 because no exemplary compound I-3-mediated reduction in PrP^{C} levels was observed in the ATP1A1 r/r cell model (FIG. 18 B). In light of the well-known preferential expression of ATP1A2 and ATP1A3 in astrocytes and neurons, respectively, if the exemplary compound I-3-dependent reduction in ATP1A2 and concomitant increase in ATP1A3 steady-state levels reflected an astrocyte-to-neuron trans-differentiation or occurred with no measurable change to the differentiation state of these cells was considered. To this end, levels of the neuron-specific class III beta tubulin (Tuj-1) and the astrocytic glial fibrillary acidic protein (GFAP) in ReN VM cells treated with exemplary compound I-3 were compared. Western blot analyses of cellular extracts, which had been adjusted for total protein, revealed that the exemplary compound I-3-mediated, ATP1A1-dependent decrease in PrP^{C} levels was not paralleled by significant changes in the levels of Tuj-1 or GFAP (FIG. 18 C). Taken together this experiment highlighted the importance of exemplary compound I-3 being able to dock into its cognate binding site on ATP1A1 for all changes in steady-state levels described here. It also suggested that the exemplary compound I-3-induced shift in the expression profile of NKA α subunits is indicative of a plasticity in the expression of these paralogous subunits, perhaps as part of a compensatory rescue for the loss of ATP1A1, rather than representing a facet of cells undergoing broad astrocyte-to-neuron reprogramming in the presence of exemplary compound I-3.

### Discussion

A systematic research program for the identification and validation of a CG that offers improved brain bioavailability relative to other molecules within this compound class is described. Because of its use for treatment for prion diseases, validation efforts focused on the potency of a lead CG, termed exemplary compound I-3, to reduce levels of PrP^{C} by targeting its next neighbor NKA in human co-cultures of neurons and astrocytes. Starting with oleandrin, a CG that has shown some promise for brain-related applications (Unal et al., 2019; Elmaci et al., 2018; Van Kanegan et al., 2016; Dunn et al., 2011), resource-intensive in vitro screens were avoided by pairing in silico modeling and BBB penetrance predictions with insights into chemically accessible derivatives. Using this pragmatic approach, a chemical space of more 270 CGs was filtered to less than ten compounds whose Rankovic MPO.v2 scores and Glide scores for binding to NKAs were predicted to equate or surpass the corresponding scores for oleandrin. Subsequent biochemical work focused on exemplary compound I-3 (4′-dehydro-oleandrin), establishing that its brain bioavailability is twofold improved over oleandrin. The compound is stable at 37° C. for extended periods of time, and the treatment of ReN VM cells with 12 nM concentrations of exemplary compound I-3 suppressed steady-state PrP^{C} levels by as much as 84% but had no impact on the overall expression of a majority of proteins in these cells. Interestingly, the biochemical analyses indicate that, in the PrP^{C} level reduction achieved with exemplary compound I-3, the compound forms a ligand-receptor complex with ATP1A1, which in turn leads to a reduction in ATP1A1 and ATP1A2 levels and an increase in ATP1A3. The CRISPR-Cas9-driven replacement of two amino acids predicted to contribute to the canonical CG binding site within ATP1A1 prevent all of these changes to the steady-state protein levels of NKAs and PrP^{C}.

The ability of certain plant and animal species to synthesize CGs represents an adaptation that serves as a defense against herbivores and predators. Because this system is ancient and predated the existence of organisms with complex brains, an efficiency to pass the blood brain barrier is unlikely to have played a role in the evolution of CGs. Instead, compounds within this class act on cells by potently breaking their electrochemical gradient, leading at least in mammals to fatal cardiac arrhythmias as the course of death. This reality represented both a challenge and opportunity for the objective to identify CGs with improved brain bioavailability; it suggested the failure of available CGs to pass efficiently into the brain may not reflect a fundamental challenge that even extended fitness selection was not able to solve but rather constituted a case of an untapped opportunity.

Exemplary compound I-3, chemically known as 4′-dehydro-oleandrin, was first reported in a patent application from 1975 (Peterson, 1975) as one of several CGs that emerged around that time from a larger drug development program of the German Beiersdorf AG. The inventor described 4′-dehydro-oleandrin as a “good cardiotonic, and particularly suitable for use as a medicament in the treatment of cardiac insufficiency”. Its oral toxicity assessed in cats was almost twofold lower than the corresponding values for oleandrin, yet its oral effectiveness—a measure determined by comparing the lethal doses of a given compound following its oral versus intravenous administration—was approximately 20% higher. To the best of the Applicant's knowledge, the only other mention of this compound in the primary literature can be found in a report from 2016, which described its synthesis next to the synthesis of other C4′-substituted oleandrin derivatives and determined that its cytotoxicity was slightly reduced relative to oleandrin (Chen et al., 2016). The authors reported an IC₅₀ of 46 nM (after 72 hours exposure of cells) toward HeLa cells. Consistent with this result, cytotoxicity was observed after 7 day treatment of ReN VM cells at exemplary compound I-3 concentrations upward of 40 nM.

Interestingly, it was observed that ReN VM cells exhibit considerable plasticity and interdependency in regard to the steady-state levels of their NKA α subunits. As levels of ATP1A1 decreased upon exemplary compound I-3 engagement, so did ATP1A2 levels in a manner that depended on the reduction in ATP1A1 levels. In contrast, ATP1A3 levels increased together with exemplary compound I-3 levels in the cell culture medium, suggesting that the levels of this a subunit are not tied to ATP1A1, and instead may offer some level of functional compensation for the loss of ATP1A1 and ATP1A2.

While the present application has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
ADZIC, M. & NEDELJKOVIC, N. (2018). Unveiling the Role of Ecto-5′-Nucleotidase/CD73 in Astrocyte Migration by Using Pharmacological Tools. Front Pharmacol 9, 153.
APERIA, A., AKKURATOV, E. E., FONTANA, J. M. & BRISMAR, H. (2016). Na+-K+-ATPase, a new class of plasma membrane receptors. Am J Physiol Cell Physiol 310(7), C491-5.
AZARIAS G, KRUUSMÄGI M, CONNOR S, AKKURATOV E E, LIU X L, LYONS D, ET AL. A specific and essential role for Na,K-ATPase α3 in neurons co-expressing α1 and α3. J Biol Chem. 808 2013;288(4):2734-43.
BAB-DINITZ, E., ALBECK, S., PELEG, Y., BRUMFELD, V., GOTTSCHALK, K. E. & KARLISH, S. J. (2009). A C-terminal lobe of the beta subunit of Na,K-ATPase and H,K-ATPase resembles cell adhesion molecules. Biochemistry 48(36), 8684-91.
BALLMER, B. A., MOOS, R., LIBERALI, P., PELKMANS, L., HORNEMANN, S. & AGUZZI, A. (2017). Modifiers of prion protein biogenesis and recycling identified by a highly parallel endocytosis kinetics assay. J Biol Chem 292(20), 8356-8368.
BARROS-BARBOSA, A. R., FERREIRINHA, F., OLIVEIRA, A., MENDES, M., LOBO, M. G., SANTOS, A., RANGEL, R., PELLETIER, J., SEVIGNY, J., CORDEIRO, J. M. & CORREIA-DE-SA, P. (2016). Adenosine A2A receptor and ecto-5′-nucleotidase/CD73 are upregulated in hippocampal astrocytes of human patients with mesial temporal lobe epilepsy (MTLE). Purinergic Signal 12(4), 719-734.
BARRY, F., BOYNTON, R., MURPHY, M., HAYNESWORTH, S. & ZAIA, J. (2001). The SH-3 and SH-4 antibodies recognize distinct epitopes on CD73 from human mesenchymal stem cells. Biochem Biophys Res Commun 289(2), 519-24.
BATRAKOVA, E. V., MILLER, D. W., LI, S., ALAKHOV, V. Y., KABANOV, A. V. & ELMQUIST, W. F. (2001). Pluronic P85 enhances the delivery of digoxin to the brain: in vitro and in vivo studies. J Pharmacol Exp Ther 296(2), 551-7.
BENNETT, C. F. (2019). Therapeutic Antisense Oligonucleotides Are Coming of Age. Annu Rev Med 70, 307-321.
BIGNAMI, A. & PALLADINI, G. (1966). Experimentally produced cerebral status spongiosus and continuous pseudorhythmic electroencephalographic discharges with a membrane-ATPase inhibitor in the rat. Nature 209(5021), 413-4.
BIGNAMI, A., PALLADINI, G., APPICCIUTOLI, L. & MACCAGNANI, F. (1967). [Experimental study of cerebral spongiosis]. Acta Neuropathol, Suppl 3:119-26.
BIGNAMI, A., PALLADINI, G. & VENTURINI, G. (1966). Effect of cardiazol on sodium-potassium-activated adenosine triphosphatase of the rat brain in vivo. Brain Res 1(4), 413-4.
BLANCO G. Na,K-ATPase subunit heterogeneity as a mechanism for tissue-specific ion regulation. Sem Nephrol. 2005;25(5):292-303
BOTELHO, A. F. M., MIRANDA, A. L. S., FREITAS, T. G., MILANI, P. F., BARRETO, T., CRUZ, J. S. & MELO, M. M. (2020). Comparative Cardiotoxicity of Low Doses of Digoxin, Ouabain, and Oleandrin. Cardiovasc Toxicol.
BRANDNER, S., ISENMANN, S., RAEBER, A., FISCHER, M., SAILER, A., KOBAYASHI, Y., MARINO, S., WEISSMANN, C. & AGUZZI, A. (1996a). Normal host prion protein necessary for scrapie-induced neurotoxicity. Nature 379(6563), 339-43.
BRANDNER, S., RAEBER, A., SAILER, A., BLATTLER, T., FISCHER, M., WEISSMANN, C. & AGUZZI, A. (1996b). Normal host prion protein (PrPC) is required for scrapie spread within the central nervous system. Proc Natl Acad Sci U S A 93(23), 13148-51.
BRISEVAC, D., ADZIC, M., LAKETA, D., PARABUCKI, A., MILOSEVIC, M., LAVRNJA, I., BJELOBABA, I., SEVIGNY, J., KIPP, M. & NEDELJKOVIC, N. (2015). Extracellular ATP Selectively Upregulates Ecto-Nucleoside Triphosphate Diphosphohydrolase 2 and Ecto-5′-Nucleotidase by Rat Cortical Astrocytes In Vitro. J Mol Neurosci 57(3), 452-62.
BUELER, H., FISCHER, M., LANG, Y., BLUETHMANN, H., LIPP, H. P., DEARMOND, S. J., PRUSINER, S. B., AGUET, M. & WEISSMANN, C. (1992). Normal development and behaviour of mice lacking the neuronal cell-surface PrP protein. Nature 356(6370), 577-82.
BUELER, H., RAEBER, A., SAILER, A., FISCHER, M., AGUZZI, A. & WEISSMANN, C. (1994). High prion and PrPSc levels but delayed onset of disease in scrapie-inoculated mice heterozygous for a disrupted PrP gene. Molecular Medicine 1(1), 19-30.
BURNSTOCK, G. & KNIGHT, G. E. (2017). Cell culture: complications due to mechanical release of ATP and activation of purinoceptors. Cell Tissue Res 370(1), 1-11.
CAMERON R, KLEIN L, SHYJAN A W, RAKIC P, LEVENSON R. Neurons and astroglia express distinct subsets of Na,K-ATPase alpha and beta subunits. Brain Res Mol Brain Res. 811 1994;21(3-4):333-43.
CHEN, H., LEI, M., MA, B., LIU, M., GUO, D., LIU, X. & HU, L. (2016). Synthesis and cytotoxicity evaluation of 4′-amino-4′-dehydroxyloleandrin derivatives. Fitoterapia 113, 85-90.
CLAUSEN, M. V., HILBERS, F. & POULSEN, H. (2017). The Structure and Function of the Na,K-ATPase Isoforms in Health and Disease. Front Physiol 8, 371.
CZERNECKI, S., GEORGOULIS, C., STEVENS, C. L. & VIJAYAKUMARAN, K. (1985). Pyridinium dichromate oxidation. Modifications enhancing its synthetic utility. Tetrahedron Letters 26(14), 1699-1702.
DEVOS, S. L., MILLER, R. L., SCHOCH, K. M., HOLMES, B. B., KEBODEAUX, C. S., WEGENER, A. J., CHEN, G., SHEN, T., TRAN, H., NICHOLS, B., ZANARDI, T. A., KORDASIEWICZ, H. B., SWAYZE, E. E., BENNETT, C. F., DIAMOND, M. I., et al. (2017). Tau reduction prevents neuronal loss and reverses pathological tau deposition and seeding in mice with tauopathy. Sci Transl Med 9(374).
DONAHUE, H. J., QU, R. W. & GENETOS, D. C. (2017). Joint diseases: from connexins to gap junctions. Nat Rev Rheumatol 14(1), 42-51.
DONATO, R., MILJAN, E. A., HINES, S. J., AOUABDI, S., POLLOCK, K., PATEL, S., EDWARDS, F. A. & SINDEN, J. D. (2007). Differential development of neuronal physiological responsiveness in two human neural stem cell lines. BMC Neurosci 8, 36.
DOSTANIC-LARSON, I., VAN HUYSSE, J. W., LORENZ, J. N. & LINGREL, J. B. (2005). The highly conserved cardiac glycoside binding site of Na,K-ATPase plays a role in blood pressure regulation. Proc Natl Acad Sci U S A 102(44), 15845-50.
DOSTANIC, I., SCHULTZ JEL, J., LORENZ, J. N. & LINGREL, J. B. (2004). The alpha 1 isoform of Na,K-ATPase regulates cardiac contractility and functionally interacts and co-localizes with the Na/Ca exchanger in heart. J Biol Chem 279(52), 54053-61.
DUNN, D. E., HE, D. N., YANG, P., JOHANSEN, M., NEWMAN, R. A. & LO, D. C. (2011). In vitro and in vivo neuroprotective activity of the cardiac glycoside oleandrin from Nerium oleander in brain slice-based stroke models. J Neurochem 119(4), 805-14.
DUTTA, S., MARKS, B. H. & SCHOENER, E. P. (1977). Accumulation of radioactive cardiac glycosides by various brain regions in relation to the dysrhythmogenic effect. Br J Pharmacol 59(1), 101-6.
EHLE, M., PATEL, C. & GIUGLIANO, R. P. (2011). Digoxin: clinical highlights: a review of digoxin and its use in contemporary medicine. Crit Pathw Cardiol 10(2), 93-8.
ELMACI, I., ALTURFAN, E. E., CENGIZ, S., OZPINAR, A. & ALTINOZ, M. A. (2018). Neuroprotective and tumoricidal activities of cardiac glycosides. Could oleandrin be a new weapon against stroke and glioblastoma? Int J Neurosci 128(9), 865-877.
FINKEL, R. S., MERCURI, E., DARRAS, B. T., CONNOLLY, A. M., KUNTZ, N. L., KIRSCHNER, J., CHIRIBOGA, C. A., SAITO, K., SERVAIS, L., TIZZANO, E., TOPALOGLU, H., TULINIUS, M., MONTES, J., GLANZMAN, A. M., BISHOP, K., et al. (2017). Nusinersen versus Sham Control in Infantile-Onset Spinal Muscular Atrophy. N Engl J Med 377(18), 1723-1732.
FLASCH, H. & HEINZ, N. (1976). [Concentration of cardiac glycosides in the heart and brain (authors transl)]. Arzneimittelforschung 26(6), 1213-6.
FORD A R, ARONSON J K, GRAHAME-SMITH D G, ROSE J A. The characteristics of the binding of 707 12-alpha-[3H]-digoxin to the membranes of intact human erythrocytes: relevance to digoxin 708 therapy. Brit J Clin Pharm. 1979;8(2):115-24.
GHODRATI, F., MEHRABIAN, M., WILLIAMS, D., HALGAS, O., BOURKAS, M. E. C., WATTS, J. C., PAI, E. F. & SCHMITT-ULMS, G. (2018). The prion protein is embedded in a molecular environment that modulates transforming growth factor β and integrin signaling. Scientific Reports 8(1), 8654.

GRAHAM, J. F., KURIAN, D., AGARWAL, S., TOOVEY, L., HUNT, L., KIRBY, L., PINHEIRO, T. J., BANNER, S. J. & GILL, A. C. (2011). Na+/K+-ATPase is present in scrapie-associated fibrils, modulates PrP misfolding in vitro and links PrP function and dysfunction. PLoS One 6(11), e26813.
GRAY, B. C., SKIPP, P., O'CONNOR, V. M. & PERRY, V. H. (2006). Increased expression of glial fibrillary acidic protein fragments and mu-calpain activation within the hippocampus of prion-infected mice. Biochem Soc Trans 34(Pt 1), 51-4.
GREEFF, K. (1981). Cardiac Glycosides. Springer-Verlag Part I.
GUPTA, R. S., CHOPRA, A. & STETSKO, D. K. (1986). Cellular basis for the species differences in sensitivity to cardiac glycosides (digitalis). J Cell Physiol 127(2), 197-206.
HACHIYA, N., KOMATA, Y., HARGUEM, S., NISHIJIMA, K. & KANEKO, K. (2011). Possible involvement of calpain-like activity in normal processing of cellular prion protein. Neurosci Lett 490(2), 150-5.
HABECK M, TOKHTAEVA E, NADAV Y, BEN ZEEV E, FERRIS S P, KAUFMAN R J, ET AL. Selective assembly of Na,K-ATPase α2β2 heterodimers in the heart: Distinct functional properties and isoform-selective inhibitors. J Biol Chem. 2016;291(44):23159-74.
HERRERA, V. L., EMANUEL, J. R., RUIZ-OPAZO, N., LEVENSON, R. & NADAL-GINARD, B. (1987). Three differentially expressed Na,K-ATPase alpha subunit isoforms: structural and functional implications. J Cell Biol 105(4), 1855-65.
HONG, D. S., HENARY, H., FALCHOOK, G. S., NAING, A., FU, S., MOULDER, S., WHELER, J. J., TSIMBERIDOU, A., DURAND, J. B., KHAN, R., YANG, P., JOHANSEN, M., NEWMAN, R. A. & KURZROCK, R. (2014). First-in-human study of pbi-05204, an oleander-derived inhibitor of akt, fgf-2, nf-kappaBeta and p70s6k, in patients with advanced solid tumors. Invest New Drugs 32(6), 1204-12.
ITOH, Y., MORIYAMA, Y., HASEGAWA, T., ENDO, T. A., TOYODA, T. & GOTOH, Y. (2013). Scratch regulates neuronal migration onset via an epithelial-mesenchymal transition-like mechanism. Nature neuroscience 16(4), 416-25.
JEON, A. H. & SCHMITT-ULMS, G. (2012). Time-controlled transcardiac perfusion crosslinking for in vivo interactome studies. Meth Mol Biol 803, 231-46.
KANG, J., KANG, N., LOVATT, D., TORRES, A., ZHAO, Z., LIN, J. & NEDERGAARD, M. (2008). Connexin 43 hemichannels are permeable to ATP. J Neurosci 28(18), 4702-11.
KARAPETYAN, Y. E., SFERRAZZA, G. F., ZHOU, M., OTTENBERG, G., SPICER, T., CHASE, P., FALLAHI, M., HODDER, P., WEISSMANN, C. & LASMEZAS, C. I. (2013). Unique drug screening approach for prion diseases identifies tacrolimus and astemizole as antiprion agents. Proc Natl Acad Sci U S A 110(17), 7044-9.
KHATRI, H. R., BHATTARAI, B., KAPLAN, W., LI, Z., LONG, M. J. C., AYE, Y. & NAGORNY, P. (2019). Modular total synthesis and cell-based anticancer activity evaluation of ouabagenin and other cardiotonic steroids with varying degrees of oxygenation. J Am Chem Soc.
KIM, Y. H., CHOI, S. H., D'AVANZO, C., HEBISCH, M., SLIWINSKI, C., BYLYKBASHI, E., WASHICOSKY, K. J., KLEE, J. B., BRÜSTLE, O., TANZI, R. E. & KIM, D. Y. (2015). A 3D human neural cell culture system for modeling Alzheimer's disease. Nature protocols 10(7), 985-1006.
KLEENE, R., LOERS, G., LANGER, J., FROBERT, Y., BUCK, F. & SCHACHNER, M. (2007). Prion protein regulates glutamate-dependent lactate transport of astrocytes. J Neurosci 27(45), 12331-40.
KORNEENKO T V, Pestov N B, Ahmad N, Okkelman I A, Dmitriev R I, Shakhparonov MI, et al. Evolutionary diversification of the BetaM interactome acquired through co-option of the ATP1B4 gene in placental mammals. Sci Rep. 2016;6:22395.
KUHLMANN, J., ERDMANN, E. & RIETBROCK, N. (1979). Distribution of cardiac glycosides in heart and brain of dogs and their affinity to the (Na++K+)-ATPase. Naunyn-Schmiedeberg's Archives of Pharmacology 307(1), 65-71.
L A, J., REED, E., CHAN, L., SMOLYANINOVA, L. V., AKOMOVA, O. A., MUTLU, G. M., ORLOV, S. N. & DULIN, N. O. (2016). Downregulation of TGF-β Receptor-2 Expression and Signaling through Inhibition of Na/K-ATPase. PLOS ONE 11(12), e0168363.
LAN, Y. L., WANG, X., LOU, J. C., XING, J. S., YU, Z. L., WANG, H., ZOU, S., MA, X. & ZHANG, B. (2018). Bufalin inhibits glioblastoma growth by promoting proteasomal degradation of the Na(+)/K(+)-ATPase alphal subunit. Biomed Pharmacother 103, 204-215.
LAPATO, A. S. & TIWARI-WOODRUFF, S. K. (2018). Connexins and pannexins: At the junction of neuro-glial homeostasis & disease. J Neurosci Res 96(1), 31-44.
LAUREN, J., GIMBEL, D. A., NYGAARD, H. B., GILBERT, J. W. & STRITTMATTER, S. M. (2009). Cellular prion protein mediates impairment of synaptic plasticity by amyloid-beta oligomers. Nature 457(7233), 1128-32.
LAURSEN, M., GREGERSEN, J. L., YATIME, L., NISSEN, P. & FEDOSOVA, N. U. (2015). Structures and characterization of digoxin- and bufalin-bound Na;-ATPase compared with the ouabain-bound complex. Proceedings of the National Academy of Sciences 112(6), 1755.
LAURSEN, M., YATIME, L., NISSEN, P. & FEDOSOVA, N. U. (2013). Crystal structure of the high-affinity Na+K+-ATPase-ouabain complex with Mg2+ bound in the cation binding site. Proc Natl Acad Sci U S A 110(27), 10958-63.
LECUONA E, LUQUIN S, AVILA J, GARCIA-SEGURA L M, MARTIN-VASALLO P. Expression of the beta 1 and beta 2(AMOG) subunits of the Na,K-ATPase in neural tissues: cellular and developmental distribution patterns. Brain Res Bull. 1996;40(3):167-74.
LEE, Y. B., DU, S., RHIM, H., LEE, E. B., MARKELONIS, G. J. & OH, T. H. (2000). Rapid increase in immunoreactivity to GFAP in astrocytes in vitro induced by acidic pH is mediated by calcium influx and calpain I. Brain Res 864(2), 220-9.
LLORENS, F., THUNE, K., SIKORSKA, B., SCHMITZ, M., TAHIR, W., FERNANDEZ-BORGES, N., CRAMM, M., GOTZMANN, N., CARMONA, M., STREICHENBERGER, N., MICHEL, U., ZAFAR, S., SCHUETZ, A. L., RAJPUT, A., ANDREOLETTI, O., et al. (2017). Altered Ca(2+) homeostasis induces Calpain-Cathepsin axis activation in sporadic Creutzfeldt-Jakob disease. Acta Neuropathol Commun 5(1), 35.
LUCCHESI, P. A. & SWEADNER, K. J. (1991). Postnatal changes in Na,K-ATPase isoform expression in rat cardiac ventricle. Conservation of biphasic ouabain affinity. J Biol Chem 266(14), 9327-31.
LUNDGREN, D. H., HWANG, S. I., WU, L. & HAN, D. K. (2010). Role of spectral counting in quantitative proteomics. Expert Rev Proteomics 7(1), 39-53.
LUPIA, M., ANGIOLINI, F., BERTALOT, G., FREDDI, S., SACHSENMEIER, K. F., CHISCI, E., KUTRYB-ZAJAC, B., CONFALONIERI, S., SMOLENSKI, R. T., GIOVANNONI, R., COLOMBO, N., BIANCHI, F. & CAVALLARO, U. (2018). CD73 Regulates Stemness and Epithelial-Mesenchymal Transition in Ovarian Cancer-Initiating Cells. Stem Cell Reports 10(4), 1412-1425.
MA, J. & LINDQUIST, S. (2001). Wild-type PrP and a mutant associated with prion disease are subject to retrograde transport and proteasome degradation. Proceedings of the National Academy of Sciences of the United States of America 98(26), 14955-60.
MALLUCCI, G., DICKINSON, A., LINEHAN, J., KLOHN, P. C., BRANDNER, S. & COLLINGE, J. (2003). Depleting neuronal PrP in prion infection prevents disease and reverses spongiosis. Science 302(5646), 871-4.
MALLUCCI, G. R., WHITE, M. D., FARMER, M., DICKINSON, A., KHATUN, H., POWELL, A. D., BRANDNER, S., JEFFERYS, J. G. & COLLINGE, J. (2007). Targeting cellular prion protein reverses early cognitive deficits and neurophysiological dysfunction in prion-infected mice. Neuron 53(3), 325-35.
MARKHAM, K., BAI, Y. & SCHMITT-ULMS, G. (2007). Co-immunoprecipitations revisited: an update on experimental concepts and their implementation for sensitive interactome investigations of endogenous proteins. Anal Bioanal Chem 389(2), 461-73.
MARTIN-VASALLO P, WETZEL R K, GARCIA-SEGURA L M, MOLINA-HOLGADO E, ARYSTARKHOVA E, 813 SWEADNER K J. Oligodendrocytes in brain and optic nerve express the beta3 subunit isoform 814 of Na,K-ATPase. Glia. 2000;31(3):206-18
MAYER, U., WAGENAAR, E., BEIJNEN, J. H., SMIT, J. W., MEIJER, D. K., VAN ASPEREN, J., BORST, P. & SCHINKEL, A. H. (1996). Substantial excretion of digoxin via the intestinal mucosa and prevention of long-term digoxin accumulation in the brain by the mdr 1a P-glycoprotein. Br J Pharmacol 119(5), 1038-44.
MAYS, C. E., KIM, C., HALDIMAN, T., VAN DER MERWE, J., LAU, A., YANG, J., GRAMS, J., DI BARI, M. A., NONNO, R., TELLING, G. C., KONG, Q., LANGEVELD, J., MCKENZIE, D., WESTAWAY, D. & SAFAR, J. G. (2014). Prion disease tempo determined by host-dependent substrate reduction. J Clin Invest 124(2), 847-58.
MAYS, C. E., VAN DER MERWE, J., KIM, C., HALDIMAN, T., MCKENZIE, D., SAFAR, J. G. & WESTAWAY, D. (2015). Prion Infectivity Plateaus and Conversion to Symptomatic Disease Originate from Falling Precursor Levels and Increased Levels of Oligomeric PrPSc Species. J Virol 89(24), 12418-26.
MAZUR, C., POWERS, B., ZASADNY, K., SULLIVAN, J. M., DIMANT, H., KAMME, F., HESTERMAN, J., MATSON, J., OESTERGAARD, M., SEAMAN, M., HOLT, R. W., QUTAISH, M., POLYAK, I., COELHO, R., GOTTUMUKKALA, V., et al. (2019). Brain pharmacology of intrathecal antisense oligonucleotides revealed through multimodal imaging. JCI Insight 4(20).
MEHRABIAN, M., BRETHOUR, D., MACISAAC, S., KIM, J. K., GUNAWARDANA, C. G., WANG, H. & SCHMITT-ULMS, G. (2014). CRISPR-Cas9-Based Knockout of the Prion Protein and Its Effect on the Proteome. PLoS One 9(12), e114594.
MEHRABIAN, M., BRETHOUR, D., WANG, H., XI, Z., ROGAEVA, E. & SCHMITT-ULMS, G. (2015). The Prion Protein Controls Polysialylation of Neural Cell Adhesion Molecule 1 during Cellular Morphogenesis. PLoS One 10(8), e0133741.
MEHRABIAN, M., HILDEBRANDT, H. & SCHMITT-ULMS, G. (2016). NCAM1 Polysialylation: The Prion Protein's Elusive Reason for Being? ASN Neuro 8(6).
MENGER, L., VACCHELLI, E., KEPP, O., EGGERMONT, A., TARTOUR, E., ZITVOGEL, L., KROEMER, G. & GALLUZZI, L. (2013). Trial watch: Cardiac glycosides and cancer therapy. Oncoimmunology 2(2), e23082.
MIJATOVIC, T., DUFRASNE, F. & KISS, R. (2012). Cardiotonic steroids-mediated targeting of the Na(+)/K(+)-ATPase to combat chemoresistant cancers. Curr Med Chem 19(5), 627-46.
MINIKEL, E. V., VALLABH, S. M., LEK, M., ESTRADA, K., SAMOCHA, K. E., SATHIRAPONGSASUTI, J. F., MCLEAN, C. Y., TUNG, J. Y., YU, L. P., GAMBETTI, P., BLEVINS, J., ZHANG, S., COHEN, Y., CHEN, W., YAMADA, M., et al. (2016). Quantifying prion disease penetrance using large population control cohorts. Sci Transl Med 8(322), 322ra9.
MINIKEL, E. V., ZHAO, H. T., LE, J., O'MOORE, J., PITSTICK, R., GRAFFAM, S., CARLSON, G. A., KAVANAUGH, M. P., KRIZ, J., KIM, J. B., MA, J., WILLE, H., AIKEN, J., MCKENZIE, D., DOH-URA, K., et al. (2020). Prion protein lowering is a disease-modifying therapy across prion disease stages, strains and endpoints. Nucleic Acids Res.
MIRANZADEH MAHABADI, H. & TAGHIBIGLOU, C. (2020). Cellular Prion Protein (PrP^{C}): Putative Interacting Partners and Consequences of the Interaction. Int J Mol Sci 21(19).
MUKAI, K., KASUYA, S., NAKAGAWA, Y., URABE, D. & INOUE, M. (2015). A convergent total synthesis of ouabagenin. Chem Sci 6(6), 3383-3387.
MURATA, K., KINOSHITA, T., ISHIKAWA, T., KURODA, K., HOSHI, M. & FUKAZAWA, Y. (2020). Region- and neuronal-subtype-specific expression of Na,K-ATPase alpha and beta subunit isoforms in the mouse brain. J Comp Neurol 528(16), 2654-2678.
NI, D., MADDEN, T. L., JOHANSEN, M., FELIX, E., HO, D. H. & NEWMAN, R. A. (2002). Murine pharmacokinetics and metabolism of oleandrin, a cytotoxic component of Nerium oleander. J Exp Ther Oncol 2(5), 278-85.
PEREIRA, G. S., WALZ, R., BONAN, C. D., BATTASTINI, A. M., IZQUIERDO, I., MARTINS, V. R., BRENTANI, R. R. & SARKIS, J. J. (2001). Changes in cortical and hippocampal ectonucleotidase activities in mice lacking cellular prion protein. Neurosci Lett 301(1), 72-4.
PETERSON, R. (1975). 4′-Dehydro-oleandrin and pharmaceutical composition thereof. U.S. Pat. No. 3,898,331. Washington, DC: U.S. Patent and Trademark Office.
POLI, G., GRANCHI, C., RIZZOLIO, F. & TUCCINARDI, T. (2020). Application of MM-PBSA Methods in Virtual Screening. Molecules 25(8).
PRICE, E. M. & LINGREL, J. B. (1988). Structure-function relationships in the Na,K-ATPase alpha subunit: site-directed mutagenesis of glutamine-111 to arginine and asparagine-122 to aspartic acid generates a ouabain-resistant enzyme. Biochemistry 27(22), 8400-8.
PRICE, E. M., RICE, D. A. & LINGREL, J. B. (1990). Structure-function studies of Na,K-ATPase. Site-directed mutagenesis of the border residues from the H1-H2 extracellular domain of the alpha subunit. J Biol Chem 265(12), 6638-41.
PRUSINER, S. B. (1998). Prions. Proc Natl Acad Sci USA 95(23), 13363-83.
RANKOVIC, Z. (2017). CNS Physicochemical Property Space Shaped by a Diverse Set of Molecules with Experimentally Determined Exposure in the Mouse Brain. J Med Chem 60(14), 5943-5954.
REDDY, M. S., ZHANG, H., PHOENIX, S. & DESLONGCHAMPS, P. (2009). Total synthesis of ouabagenin and ouabain. Chem Asian J 4(5), 725-41.
RENATA, H., ZHOU, Q. & BARAN, P. S. (2013). Strategic redox relay enables a scalable synthesis of ouabagenin, a bioactive cardenolide. Science 339(6115), 59-63.
RENKAWEK, K., RENIER, W. O., DE PONT, J. J., VOGELS, O. J. & GABREELS, F. J. (1992). Neonatal status convulsivus, spongiform encephalopathy, and low activity of Na+/K(+)-ATPase in the brain. Epilepsia 33(1), 58-64.
REPKE, K., EST, M. & PORTIUS, H. J. (1965). [On the cause of species differences in digitalis sensitivity]. Biochem Pharmacol 14(12), 1785-802.
RUTISHAUSER, D., MERTZ, K. D., MOOS, R., BRUNNER, E., RULICKE, T., CALELLA, A. M. & AGUZZI, A. (2009). The comprehensive native interactome of a fully functional tagged prion protein. PloS One 4(2), e4446.
SAILER, A., BUELER, H., FISCHER, M., AGUZZI, A. & WEISSMANN, C. (1994). No propagation of prions in mice devoid of PrP. Cell 77(7), 967-8.
SANTUCCIONE, A., SYTNYK, V., LESHCHYNS'KA, I. & SCHACHNER, M. (2005). Prion protein recruits its neuronal receptor NCAM to lipid rafts to activate p59fyn and to enhance neurite outgrowth. J Cell Biol 169(2), 341-54.
SCHATZMANN, H. J. (1953). Herzglykoside als Hemmstoffe für den aktiven Kalium- and Natriumtransport durch die Erythrocytenmembran. (Cardiac glycosides as inhibitors for the active potassium and sodium transport across the red cell membrane.) HeIv. Physiol. Pharmacol. Acta 11, 346-354.
SCHINKEL, A. H., MAYER, U., WAGENAAR, E., MOL, C. A., VAN DEEMTER, L., SMIT, J. J., VAN DER VALK, M. A., VOORDOUW, A. C., SPITS, H., VAN TELLINGEN, O., ZIJLMANS, J. M., FIBBE, W. E. & BORST, P. (1997). Normal viability and altered pharmacokinetics in mice lacking mdrl-type (drug-transporting) P-glycoproteins. Proc Natl Acad Sci U S A 94(8), 4028-33.
SCHMALZING G, RUHL K, GLOOR S M. Isoform-specific interactions of Na,K-ATPase subunits are mediated via extracellular domains and carbohydrates. Proc Natl Acad Sci U S A. 805 1997;94(4):1136-41.
SCHMITT-ULMS, G., HANSEN, K., LIU, J., COWDREY, C., YANG, J., DEARMOND, S. J., COHEN, F. E., PRUSINER, S. B. & BALDWIN, M. A. (2004). Time-controlled transcardiac perfusion cross-linking for the study of protein interactions in complex tissues. Nature Biotechnology 22(6), 724-31.
SCHMITT-ULMS, G., LEGNAME, G., BALDWIN, M. A., BALL, H. L., BRADON, N., BOSQUE, P. J., CROSSIN, K. L., EDELMAN, G. M., DEARMOND, S. J., COHEN, F. E. & PRUSINER, S. B. (2001). Binding of neural cell adhesion molecules (N-CAMs) to the cellular prion protein. Journal of Molecular Biology 314(5), 1209-25.
SHAHBAZI, A., SAFA, M., ALIKARAMI, F., KARGOZAR, S., ASADI, M. H., JOGHATAEI, M. T. & SOLEIMANI, M. (2016). Rapid Induction of Neural Differentiation in Human Umbilical Cord Matrix Mesenchymal Stem Cells by cAMP-elevating Agents. Int J Mol Cell Med 5(3), 167-177.
SHINODA, T., OGAWA, H., CORNELIUS, F. & TOYOSHIMA, C. (2009). Crystal structure of the sodium-potassium pump at 2.4 A resolution. Nature 459(7245), 446-50.
SHYNG, S. L., HUBER, M. T. & HARRIS, D. A. (1993). A prion protein cycles between the cell surface and an endocytic compartment in cultured neuroblastoma cells. J Biol Chem 268(21), 15922-8.
SILBER, B. M., GEVER, J. R., RAO, S., LI, Z., RENSLO, A. R., WIDJAJA, K., WONG, C., GILES, K., FREYMAN, Y., ELEPANO, M., IRWIN, J. J., JACOBSON, M. P. & PRUSINER, S. B. (2014). Novel compounds lowering the cellular isoform of the human prion protein in cultured human cells. Bioorg Med Chem 22(6), 1960-72.
SLAPSAK, U., SALZANO, G., AMIN, L., ABSKHARON, R. N., ILC, G., ZUPANCIC, B., BILJAN, I., PLAVEC, J., GIACHIN, G. & LEGNAME, G. (2016). The N Terminus of the Prion Protein Mediates Functional Interactions with the Neuronal Cell Adhesion Molecule (NCAM) Fibronectin Domain. J Biol Chem 291(42), 21857-21868.
SOLHEID, C. & PALLADINI, G. (1974). Morphological changes induced by ouabain in normal and neoplastic human glia in monolayer culture. Acta Neuropathol 28(3), 253-60.
TASKAR, K. S., MARIAPPAN, T. T., KURAWATTIMATH, V., SINGH GAUTAM, S., RADHAKRISHNA MULLAPUDI, T. V., SRIDHAR, S. K., KALLEM, R. R., MARATHE, P. & MANDLEKAR, S. (2017). Unmasking the Role of Uptake Transporters for Digoxin Uptake Across the Barriers of the Central Nervous System in Rat. J Cent Nery Syst Dis 9, 1179573517693596.
TATZELT, J. & SCHATZL, H. M. (2007). Molecular basis of cerebral neurodegeneration in prion diseases. Febs j 274(3), 606-11.
THOMPSON, L. F., ELTZSCHIG, H. K., IBLA, J. C., VAN DE WIELE, C. J., RESTA, R., MOROTE-GARCIA, J. C. & COLGAN, S. P. (2004). Crucial role for ecto-5′-nucleotidase (CD73) in vascular leakage during hypoxia. J Exp Med 200(11), 1395-405.
TOBIN T, HENDERSON R, Sen A K. Species and tissue differences in the rate of dissociation of 705 ouabain from (Na++K+)-ATPase. Biochim Biophys Acta. 1972;274(2):551-5.
TOKHTAEVA E, Clifford R J, Kaplan J H, Sachs G, Vagin O. Subunit isoform selectivity in assembly of Na,K-ATPase alpha-beta heterodimers. J Biol Chem. 2012;287(31):26115-25.
TURAN, N., AKGUN-DAR, K., KURUCA, S. E., KILICASLAN-AYNA, T., SEYHAN, V. G., ATASEVER, B., MERICLI, F. & CARIN, M. (2006). Cytotoxic effects of leaf, stem and root extracts of Nerium oleander on leukemia cell lines and role of the p-glycoprotein in this effect. J Exp Ther Oncol 6(1), 31-8.
UNAL, I., CALISKAN-AK, E., USTUNDAG, U. V., ATES, P. S., ALTURFAN, A. A., ALTINOZ, M. A., ELMACI, I. & EMEKLI-ALTURFAN, E. (2019). Neuroprotective Effects of Mitoquinone and Oleandrin on Parkinson's Disease Model in Zebrafish. Int J Neurosci, 1-14.
VAN KANEGAN, M. J., DUNN, D. E., KALTENBACH, L. S., SHAH, B., HE, D. N., MCCOY, D. D., YANG, P., PENG, J., SHEN, L., DU, L., CICHEWICZ, R. H., NEWMAN, R. A. & LO, D. C. (2016). Dual activities of the anti-cancer drug candidate PBI-05204 provide neuroprotection in brain slice models for neurodegenerative diseases and stroke. Sci Rep 6, 25626.
VENTURINI, G. & PALLADINI, G. (1973). ATPase activity, sodium and potassium content in guinea pig cortex after ouabain treatment in vivo. J Neurochem 20(1), 237-9.
WALLICK, E. T., PITTS, B. J., LANE, L. K. & SCHWARTZ, A. (1980). A kinetic comparison of cardiac glycoside interactions with Na+,K+-ATPases from skeletal and cardiac muscle and from kidney. Arch Biochem Biophys 202(2), 442-9.
WANG, J. K. T., PORTBURY, S., THOMAS, M. B., BARNEY, S., RICCA, D. J., MORRIS, D. L., WARNER, D. S. & LO, D. C. (2006). Cardiac glycosides provide neuroprotection against ischemic stroke: discovery by a brain slice-based compound screening platform. Proc Natl Acad Sci U S A 103(27), 10461-10466.
WANG, X., WILLIAMS, D., MÜLLER, I., LEMIEUX, M., DUKART, R., MAIA, I. B. L., WANG, H., WOERMAN, A. L. & SCHMITT-ULMS, G. (2019). Tau interactome analyses in CRISPR-Cas9 engineered neuronal cells reveal ATPase-dependent binding of wild-type but not P301L Tau to non-muscle myosins. Scientific Reports 9(1), 16238.
WANSAPURA, A. N., LASKO, V. M., LINGREL, J. B. & LORENZ, J. N. (2011). Mice expressing ouabain-sensitive alphal-Na,K-ATPase have increased susceptibility to pressure overload-induced cardiac hypertrophy. Am J Physiol Heart Circ Physiol 300(1), H347-55.
WATTS, A. G., SANCHEZ-WATTS, G., EMANUEL, J. R. & LEVENSON, R. (1991). Cell-specific expression of mRNAs encoding Na+,K(+)-ATPase alpha- and beta-subunit isoforms within the rat central nervous system. Proc Natl Acad Sci U S A 88(16), 7425-9.
WATTS, J. C., DRISALDI, B., NG, V., YANG, J., STROME, B., HORNE, P., SY, M. S., YOONG, L., YOUNG, R., MASTRANGELO, P., BERGERON, C., FRASER, P. E., CARLSON, G. A., MOUNT, H. T., SCHMITT-ULMS, G., et al. (2007). The CNS glycoprotein Shadoo has PrP(C)-like protective properties and displays reduced levels in prion infections. EMBO J 26(17), 4038-50.
WATTS, J. C., HUO, H., BAI, Y., EHSANI, S., JEON, A. H., SHI, T., DAUDE, N., LAU, A., YOUNG, R., XU, L., CARLSON, G. A., WILLIAMS, D., WESTAWAY, D. & SCHMITT-ULMS, G. (2009). Interactome analyses identify ties of PrP and its mammalian paralogs to oligomannosidic N-glycans and endoplasmic reticulum-derived chaperones. PLoS Pathogens 5(10), e1000608.
WOO, A. L., JAMES, P. F. & LINGREL, J. B. (2002). Roles of the Na,K-ATPase alpha4 isoform and the Na+/H+exchanger in sperm motility. Mol Reprod Dev 62(3), 348-56.
XIONG, L., WEN, Y., MIAO, X. & YANG, Z. (2014). NTSE and FcGBP as key regulators of TGF-1-induced epithelial-mesenchymal transition (EMT) are associated with tumor progression and survival of patients with gallbladder cancer. Cell Tissue Res 355(2), 365-74.
YADAVALLI, R., GUTTMANN, R. P., SEWARD, T., CENTERS, A. P., WILLIAMSON, R. A. & TELLING, G. C. (2004). Calpain-dependent endoproteolytic cleavage of PrPSc modulates scrapie prion propagation. The Journal of biological chemistry 279(21), 21948-56.
YATIME, L., BUCH-PEDERSEN, M. J., MUSGAARD, M., MORTH, J. P., LUND WINTHER, A. M., PEDERSEN, B. P., OLESEN, C., ANDERSEN, J. P., VILSEN, B., SCHIOTT, B., PALMGREN, M. G., MOLLER, J. V., NISSEN, P. & FEDOSOVA, N. (2009). P-type ATPases as drug targets: tools for medicine and science. Biochim Biophys Acta 1787(4), 207-20.
YEDIDIA, Y., HORONCHIK, L., TZABAN, S., YANAI, A. & TARABOULOS, A. (2001). Proteasomes and ubiquitin are involved in the turnover of the wild-type prion protein. The EMBO journal 20(19), 5383-91.
YEGUTKIN, G. G. (2008). Nucleotide- and nucleoside-converting ectoenzymes: Important modulators of purinergic signalling cascade. Biochim Biophys Acta 1783(5), 673-94.
YU, J., LIAO, X., LI, L., LV, L., ZHI, X., YU, J. & ZHOU, P. (2017). A preliminary study of the role of extracellular -5′- nucleotidase in breast cancer stem cells and epithelial-mesenchymal transition. In Vitro Cell Dev Biol Anim 53(2), 132-140.
ZHANG, H., SRIDHAR REDDY, M., PHOENIX, S. & DESLONGCHAMPS, P. (2008). Total synthesis of ouabagenin and ouabain. Angew Chem Int Ed Engl 47(7), 1272-5.
ZHANG, Y., CHEN, X., ZHOU, Y., HOU, J., LONG, H., ZHANG, Z., LEI, M. & WU, W. (2020). Synthesis of oleandrin derivatives and their cytotoxic activity. Steroids, 108650.
ZHOU, C., YU, F., ZENG, P., ZHANG, T., HUANG, H., CHEN, W. & WU, B. (2019). Circadian sensitivity to the cardiac glycoside oleandrin is associated with diurnal intestinal P-glycoprotein expression. Biochem Pharmacol 169, 113622.
ZIESKE, L. R. (2006). A perspective on the use of iTRAQ reagent technology for protein complex and profiling studies. Journal of experimental botany 57(7), 1501-8.

1-3. (canceled)

4. A method for treating and/or preventing prion diseases and/or brain diseases, disorders or conditions that benefit from reduced levels of the cellular prion protein (PrP^{C}) and/or altered levels of NKA alpha subunits comprising administering an effective amount of one or more agents that alter levels of one or more NKA alpha subunits, in a subject in need thereof, wherein each NKA alpha subunit with altered levels is a different paralog, and
wherein the one or more agents that alter levels of one or more NKA alpha subunits are one or more compounds of Formula I, and/or a pharmaceutically acceptable salt and/or solvate thereof: 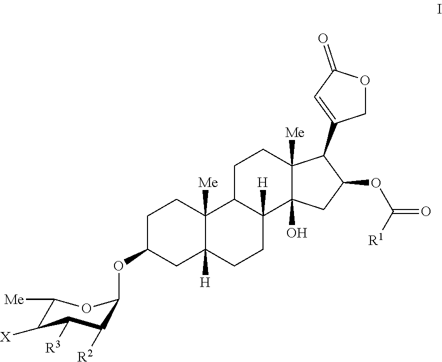
wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH, NH₂, NHC₁₋₄alkyl, and N(C₁₋₄alkyl)₂.

5. (canceled)

6. The method of claim 4, wherein R¹ is selected from CH₃, CF₃, CF₂H, CFH₂, CH₂CH₃, CF₂CF₃, CH₂CF₂H, CH₂CF₂H, CH(CH₃)₂, CF(CF₃)₂, C(CF₃)₃, and C(CH₃)₂.

7. (canceled)

8. The method of claim 6, wherein R¹ is selected from CH₃ and CF₃.

9. (canceled)

10. The method of claim 4, wherein R² and R³ are independently selected from H, OCH₃, OCF₃, OCF₂H, OCFH₂, OCH₂CH₃, OCH₂CF₂H, OCH₂CF₂H, OCF₂CF₃, OCH(CH₃)₂, OCF(CF₃)₂, OC(CF₃)₃, and OC(CH₃)₂.

11. (canceled)

12. The method of claim 10, wherein R² is selected from H, OCH₃ and OCF₃ and R³ is selected from OCH₃, OCF₃, OCH(CH₃)₂ and OCF(CF₃)₂.

13. (canceled)

14. The method of claim 4, wherein X is selected from ═O, ═NH, NH₂, NHCH₃, and N(CH₃)₂.

15. (canceled)

16. The method of claim 14, wherein X is ═O.

17. The method of clam 14, wherein X is NH₂.

18. The method of claim 4, wherein the one or more compounds of Formula I are selected from the compounds listed below:

| Compound | |
|---|---|
| I.D | Structures |
| I-1 | 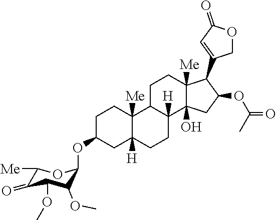 |
| I-2 | 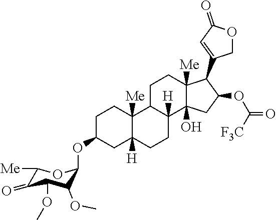 |
| I-3 | 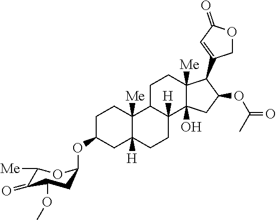 |
| I-4 | 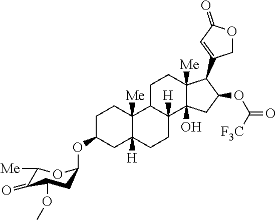 |
| I-5 | 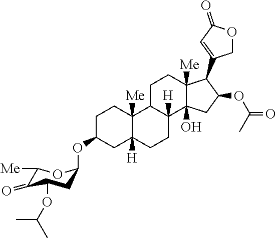 |
| I-6 | 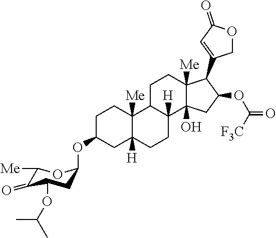 |
| I-7 | 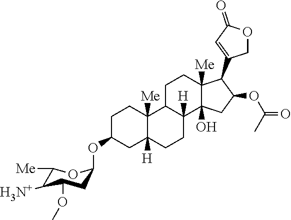 |
| I-8 | 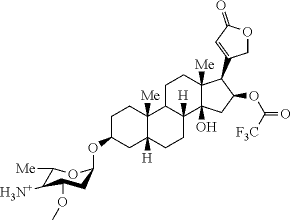 |

and/or pharmaceutically acceptable salts and/or solvates thereof.

19-22. (canceled)

23. The method of claim 4, wherein the one or more agents alter levels of one or more NKA alpha subunit paralogs selected from ATP1A1, ATP1A2 and/or ATP1A3 and the conditions associated with altered levels of NKA alpha subunits are brain diseases, disorders or conditions associated with altered ATP1A1, ATP1A2 and/or ATP1A3 levels.

24. The method of claim 23, wherein the brain diseases, disorders or conditions associated with altered ATP1A1, ATP1A2 and/or ATP1A3 levels are selected from rapid-onset dystonia parkinsonism, hemiplegia, autosomal dominant cone-rod dystrophy, Angelman's syndrome, SOD-1 forms of amyotrophic lateral sclerosis and/or forms of ataxia, epilepsy and/or mania.

25. (canceled)

26. (canceled)

27. The method of claim 4, wherein the prion disease is selected from scrapie in sheep, chronic wasting disease in deer elk and moose, bovine spongiform encephalopathy in cattle, and Creutzfeldt-Jakob disease, Gerstmann-Sträussler-Scheinker syndrome and fatal familial insomnia in humans.

28. The method of claim 4, wherein the brain disease that benefits from reduced levels of the cellular prion protein (PrP^{C}) is Alzheimer's disease.

29. (canceled)

30. (canceled)

31. A compound of Formula I-A or a pharmaceutically acceptable salt and/or solvate thereof: 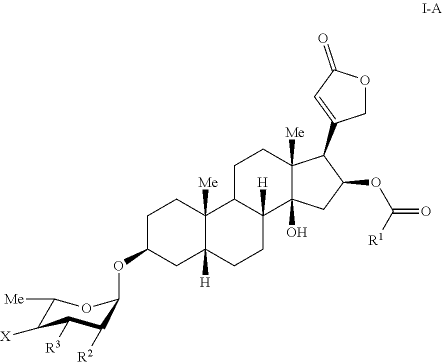
wherein
R¹ is selected from C₁₋₄alkyl and C₁₋₄fluoroalkyl;
R² and R³ are independently selected from H, OC₁₋₄alkyl and OC₁₋₄fluoroalkyl; and
X is selected from ═O, ═NH and NH₂;
provided when X is ═O, R² is H and R³ is OCH₃, then R¹ is not CH₃ or CF₃; and
provided when X is NH₂, R² is H and R³ is OCH₃, then R¹ is not CH₃.

32. The compound of claim 31, wherein R² and R³ are independently selected from H, OCH₃, OCF₃, OCF₂H, OCFH₂, OCH₂CH₃, OCH₂CF₂H, OCH₂CF₂H, OCF₂CF₃, OCH(CH₃)₂, OCF(CF₃)₂, OC(CF₃)₃, and OC(CH₃)₂.

33. (canceled)

34. The compound of claim 32, wherein R² is selected from H, OCH₃ and OCF₃, and R³ is selected from OCH₃, OCF₃, OCH(CH₃)₂ and OCF(CF₃)₂.

35. The compound of claim, 34 wherein R² is H.

36. (canceled)

37. The compound of claim 31, wherein X is ═O.

38. The compound of claim 31, wherein X is NH₂.

39. (canceled)

40. The compound of claim 31, wherein the compound of Formula I-A is selected from I-1, I-2, I-5, I-6 and I-8 or a pharmaceutically acceptable salt and/or solvate thereof.

| Compound | |
|---|---|
| I.D | Structures |
| I-1 | 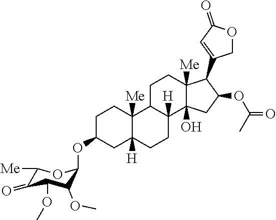 |
| I-2 | 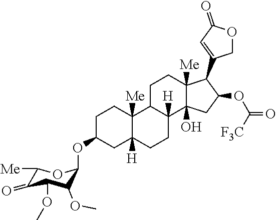 |
| I-5 | 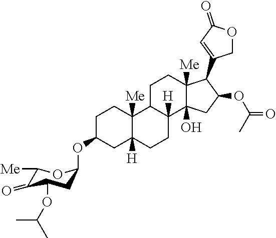 |
| I-6 | 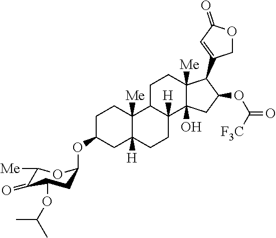 |
| I-8 | 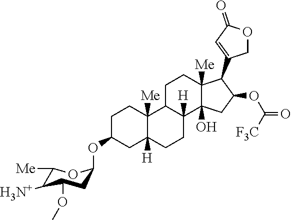 |
